(19) United States
(12) Patent Application Publication
(10) Pub. No.: US 2024/0101566 A1
(43) Pub. Date: Mar. 28, 2024

(54) TRICYCLIC UBIQUITIN SPECIFIC PROTEASE 1 INHIBITOR AND USE THEREOF

(71) Applicant: SHANDONG XUANZHU PHARMA CO., LTD., Jinan (CN)

(72) Inventors: Bin LIU, Jinan (CN); Bo CHEN, Jinan (CN)

(21) Appl. No.: 18/499,843

(22) Filed: Nov. 1, 2023

Related U.S. Application Data
(63) Continuation of application No. PCT/CN2022/089956, filed on Apr. 28, 2022.

(30) Foreign Application Priority Data
May 3, 2021 (CN) 202110486972.6
Jul. 16, 2021 (CN) 202110806606.4
Dec. 13, 2021 (CN) 202111514025.X
Jan. 19, 2022 (CN) 202210062340.1

(51) Int. Cl. C07D 487/06

(52) U.S. Cl. CPC C07D 487/06; C07B 2200/05

(57) ABSTRACT

Disclosed is a tricyclic ubiquitin specific protease 1 inhibitor compound, a pharmaceutically acceptable salt thereof, an ester thereof, a deuterated compound or a stereoisomer thereof; a pharmaceutical composition and a formulation containing the compound, the pharmaceutically acceptable salt thereof, the ester thereof, the deuterated compound or the stereoisomer thereof; a method for preparing the compound, the pharmaceutically acceptable salt thereof, the ester thereof, the deuterated compound or the stereoisomer thereof; and use of the compound, the pharmaceutically acceptable salt thereof, the ester thereof, the deuterated compound or the stereoisomer thereof in the manufacture of a medicament for treating and/or preventing a disease mediated by USP1 and a disease related thereto.

### CROSS-REFERENCE TO RELATED APPLICATIONS

The present disclosure is a continuation of International Application No. PCT/CN2022/089956, filed on Apr. 28, 2022, which claims priority to Chinese Patent Application No. 202110486972.6, filed on May 3, 2021, Chinese Patent Application No. 202110806606.4, filed on Jul. 16, 2021, Chinese Patent Application No. 202111514025.X, filed on Dec. 13, 2021, and Chinese Patent Application No. 202210062340.1, filed on Jan. 19, 2022. All of the aforementioned patent applications are hereby incorporated by reference in their entireties.

### TECHNICAL FIELD

The disclosure relates to the technical field of medicine, and particularly relates to a tricyclic ubiquitin specific protease 1 inhibitor compound, a pharmaceutically acceptable salt thereof, an ester thereof, a deuterated compound or a stereoisomer thereof; a pharmaceutical composition and a formulation containing the compound, the pharmaceutically acceptable salt thereof, the ester thereof, the deuterated compound or the stereoisomer thereof; a method for preparing the compound, the pharmaceutically acceptable salt thereof, the ester thereof, the deuterated compound or the stereoisomer thereof; and a use of the compound, the pharmaceutically acceptable salt thereof, the ester thereof, the deuterated compound or the stereoisomer thereof in the manufacture of a medicament for treating and/or preventing a disease mediated by USP1 and a disease related thereto.

### BACKGROUND

There are many relevant targets in the occurrence and development of tumors. Deubiquitinating enzyme DUB encodes more than 100 human genes and is divided into 6 families, where ubiquitin-specific protease (USP) contains 50 members and is the largest family of DUB. Ubiquitination is a reversible process. DUB acts on ubiquitin-protease systems, lysing an isopeptide bond between lysine and a C-terminus of UBQ (Ubiquitin), affecting cell proliferation, cycle, apoptosis, DNA damage response, tumor suppression, occurrence and metastasis.

USP1 (Ubiquitin specific protease) is a member of the USP family, and is a cysteine isopeptidase with a triple structure containing Cys90, His593 and Asp751. Human USP1 gene was cloned in 1998, encoding one protein with 785 amino acids. In a normal state, USP1 is relatively inactive. After combining with UAF1 (USP1-associated factor 1, one cofactor containing WD40(Conseryed sequence of 40 amino acids ending with tryptophan and aspartic acid) repeats and regulating USP1 activity) to form a heterodimeric complex, USP1 is activated to play a role of deubiquitinating enzyme, stabilize a replication fork and locate in a nucleus.

USP1 is highly expressed in cancers such as breast cancer and ovarian cancer, and the expression of USP1 is also increased in other cancers. The overexpression of USP1 is related to breast/ovarian cancer BRCA1 deficiency. USP1 deubiquitously participates in various processes related to cancer, and acts on pathways such as Fanconi anemia (FA), Transdamage DNA Synthesis (TLS), cell differentiation, etc. In FA, USP1 deubiquitinates Fanconi anabema group D2 Protein (FANCD2). In TLS, USP1 deubiquitinates Proliferating cell nuclear antigen (PCNA). In cell differentiation, USP1 affects ubiquitination of DNA binding protein inhibitor family (ID), and regulates cell proliferation and differentiation.

These DNA damage response (DDR) pathways are critical to repair of DNA damage induced by DNA cross-linking agents (such as cisplatin and ultraviolet radiation, etc.). In the TLS pathway, PCNA affected by USP1 together with USP1/UAF1 and BRCA1/2 participates in DNA break repair. After stagnation of the replication fork, RAD18-mediated PCNA mono-ubiquitination promotes conversion of PCNA from replication-type polymerase (pol δ/ε) to TLS polymerase (such as POLLK), after bypassing the lesion by the TLS polymerase, USP1 deubiquitinates the PCNA again and promotes PCNA binding to convert back to replication polymerase Inhibiting USP1 leads to instability of the replication fork and has synthetic lethality with BRCA mutations.

A USP1 inhibitor inhibits DNA break repair jointly participated by PCNA with USP1/UAF1 and BRCA1/2, making the replication fork unstable. Therefore, using a small molecule inhibitor to inhibit USP1 has the potential for the treatment of cancers and other diseases, and has not been commercialized or clinically developed.

### SUMMARY

Therefore, the present disclosure aims at providing a tricyclic ubiquitin specific protease 1 inhibitor and use thereof. The specific technical solutions are as follows.

Solution 1: the present disclosure first provides a compound of general formula (I), or a pharmaceutically acceptable salt, ester, deuterated compound or stereoisomer thereof;

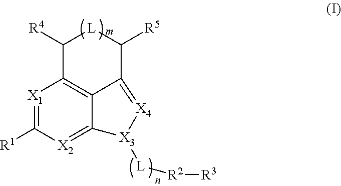

wherein:

X₁, X₂, X₃ and X₄ are each independently selected from N or CR^{a};
R¹, R² and R³ are each independently selected from 3-12 membered cycloalkyl, 3-12 membered heterocyclyl, 6-10 membered aryl or 5-12 membered heteroaryl, each of which is optionally substituted by one or more Q₁;
R⁴ and R⁵ are each independently selected from deuterium, hydrogen, carboxyl, cyano, nitro, amino, halogen, C₂₋₆ alkenyl, C₂₋₆ alkynyl, or from C₁₋₆ alkyl, C₁₋₆ alkoxy, C₁₋₆ alkylamino, di(C₁₋₆ alkyl)amino, halogenated C₁₋₆ alkyl, hydroxy C₁₋₆ alkyl, amino C₁₋₆ alkyl, carboxyl C₁₋₆ alkyl or halogenated C₁₋₆ alkoxy, each of which is optionally deuterated;
each Q₁ is independently selected from deuterium, halogen, cyano, carboxyl, hydroxyl, amino, nitro, sulfonamido, or from C₁₋₆ alkylamino, di(C₁₋₆ alkyl)amino, halogenated C₁₋₆ alkyl, halogenated C₁₋₆ alkoxy, hydroxy C₁₋₆ alkyl, amino C₁₋₆ alkyl, carboxyl C₁₋₆ alkyl, C₁₋₆ alkylcarbonyl, C₁₋₆ alkoxycarbonyl, C₁₋₆ alkylaminoacyl, C₁₋₆ alkylamido, C₁₋₆ alkylsulfonyl,C₁₋₆ alkylsulfonamido, C₁₋₆ alkylaminosulfonyl, —(L)ₘ-C₁₋₆ alkyl, —(L)ₘ-C₂₋₆ alkenyl, —(L)ₘ-C₂₋₆ alkynyl, —(L)ₘ-C₁₋₆ alkoxy, —(L)ₘ-6-10 membered aryl, —(L)ₘ-5-12 membered heteroaryl, —(L)ₘ-3-8 membered cycloalkyl or —(L)ₘ-3-8 membered heterocyclyl, each of which is optionally substituted by 1-4 substituents Q₂; and each Q₂ is independently selected from deuterium, halogen, carboxyl, hydroxyl, cyano, nitro, amino, C₁₋₆ alkyl, hydroxy C₁₋₆ alkyl, carboxyl C₁₋₆ alkyl, C₁₋₆ alkylamino, di(C₁₋₆ alkyl)amino, —CO-C₁₋₆ alkylene-NH₂, —CO-C₁₋₆ alkyl, C₁₋₆ alkoxy, halogenated C₁₋₆ alkyl and halogenated C₁₋₆ alkoxy;
each L is independently selected from —CO—, —O—, —S—, —SO—, —S(O)₂—, —NR^{c}— or —CR^{a}R^{b}—;
each R^{a} and each R^{b} are each independently selected from deuterium, hydrogen, halogen, amino, hydroxyl, carboxyl, cyano, C₁₋₆ alkyl, C₂₋₆ alkenyl, C₂₋₆ alkynyl, or from C₁₋₆ alkyl, C₁₋₆ alkoxy, C₁₋₆ alkylamino, di(C₁₋₆ alkyl)amino, C₁₋₆ alkylaminoacyl, C₁₋₆ alkylamido, C₁₋₆ alkylsulfonamido, C₁₋₆ alkylaminosulfonyl, halogenated C₁₋₆ alkyl, halogenated C₁₋₆ alkoxy, hydroxy C₁₋₆ alkyl, amino C₁₋₆ alkyl or carboxyl C₁₋₆ alkyl, each of which is optionally deuterated;
each R^{c} is independently selected from deuterium, hydrogen, or from C₁₋₆ alkyl, halogenated C₁₋₆ alkyl, halogenated C₁₋₆ alkoxy, hydroxy C₁₋₆ alkyl, amino C₁₋₆ alkyl and carboxyl C₁₋₆ alkyl, each of which is optionally deuterated;
each m is independently an integer of 0-3; and
each n is independently an integer of 0-6.

Solution 2: the compound or the pharmaceutically acceptable salt, ester, deuterated compound or stereoisomer thereof according to the solution 1, wherein:
X₁, X₂, X₃ and X₄ are each independently selected from N or CR^{a};
R¹, R² and R³ are each independently selected from 5-8 membered cycloalkyl, 3-8 membered heterocyclyl, phenyl or 5-6 membered heteroaryl, each of which is optionally substituted by one or more Q₁;
R⁴ and R⁵ are each independently selected from deuterium, hydrogen, cyano, halogen, or from C₁₋₆ alkyl, C₁₋₆ alkoxy, C₁₋₆ alkylamino, di(C₁₋₆ alkyl)amino, halogenated C₁₋₆ alkyl, hydroxy C₁₋₆ alkyl, amino C₁₋₆ alkyl, carboxyl C₁₋₆ alkyl or halogenated C₁₋₆ alkoxy, each of which is optionally deuterated;
each Q₁ is independently selected from deuterium, halogen, cyano, or from C₁₋₆ alkoxy, C₁₋₆ alkylamino, di(C₁₋₆ alkyl)amino, halogenated C₁₋₆ alkyl, halogenated C₁₋₆ alkoxy, hydroxy C₁₋₆ alkyl, amino C₁₋₆ alkyl, carboxyl C₁₋₆ alkyl, —(L)ₘ-C₁₋₆ alkyl, —(L)ₘ-3-6 membered cycloalkyl or —(L)ₘ-3-6 membered heterocyclyl which are optionally substituted by 1-4 substituents Q₂; and each Q₂ is independently selected from deuterium, halogen, carboxyl, hydroxyl, cyano, nitro, amino, C₁₋₆ alkyl, hydroxy C₁₋₆ alkyl, carboxyl C₁₋₆ alkyl, C₁₋₆ alkylamino, di(C₁₋₆ alkyl)amino, C₁₋₆ alkoxy, halogenated C₁₋₆ alkyl and halogenated C₁₋₆ alkoxy;
each L is independently selected from —CO—, —O—, —NR^{c}— or —CR^{a}R^{b}—;
each R^{a} and each R^{b} are each independently selected from deuterium, hydrogen, or from C₁₋₆ alkyl, C₁₋₆ alkoxy, C₁₋₆ alkylamino, di(C₁₋₆ alkyl)amino, halogenated C₁₋₆ alkyl, halogenated C₁₋₆ alkoxy, hydroxy C₁₋₆ alkyl, amino C₁₋₆ alkyl and carboxyl C₁₋₆ alkyl, each of which is optionally deuterated;
each R^{c} is independently selected from deuterium, hydrogen, or from C₁₋₆ alkyl, halogenated C₁₋₆ alkyl and halogenated C₁₋₆ alkoxy, each of which is optionally deuterated;
each m is independently an integer of 0-2; and
each n is independently an integer of 0-5.

Solution 3: the compound or the pharmaceutically acceptable salt, ester, deuterated compound or stereoisomer thereof according to the solution 1 or 2, wherein:
X₁, X₂, X₃ and X₄ are each independently selected from N;
R¹, R² and R³ are each independently selected from phenyl or 5-6 membered heteroaryl optionally substituted by 1-4 Q₁;
R⁴ and R⁵ are each independently selected from deuterium, hydrogen, or from C₁₋₄ alkyl, C₁₋₄ alkoxy, C₁₋₄ alkylamino, di(C₁₋₄ alkyl)amino, halogenated C₁₋₄ alkyl, hydroxy C₁₋₄ alkyl, amino C₁₋₄ alkyl, carboxyl C₁₋₄ alkyl or halogenated C₁₋₄ alkoxy, each of which is optionally deuterated;
each Q₁ is independently selected from deuterium, halogen, or from C₁₋₄ alkoxy, C₁₋₄ alkylamino, di(C₁₋₄ alkyl)amino, halogenated C₁₋₄ alkyl, halogenated C₁₋₄ alkoxy, hydroxy C₁₋₄ alkyl, amino C₁₋₄ alkyl, carboxyl C₁₋₄ alkyl, —(L)ₘ-C₁₋₄ alkyl, or —(L)ₘ-3-6 membered cycloalkyl, each of which is optionally substituted by 1-3 substituents Q₂; and each Q₂ is independently selected from deuterium, halogen, carboxyl, hydroxyl, cyano, nitro, amino, C₁₋₄ alkyl, hydroxy C₁₋₄ alkyl, carboxyl C₁₋₄ alkyl, C₁₋₄ alkylamino, di(C₁₋₄ alkyl)amino, C₁₋₄ alkoxy, halogenated C₁₋₄ alkyl and halogenated C₁₋₄ alkoxy;
each L is independently selected from —CR^{a}R^{b}—or —O—;
each R^{a} and each R^{b} are each independently selected from deuterium, hydrogen, or from C₁₋₄ alkyl, C₁₋₄ alkoxy, C₁₋₄ alkylamino, di(C₁₋₄ alkyl)amino, halogenated C₁₋₄ alkyl, halogenated C₁₋₄ alkoxy, hydroxy C₁₋₄ alkyl, amino C₁₋₄ alkyl and carboxyl C₁₋₄ alkyl, each of which is optionally deuterated;
each R^{c} is independently selected from deuterium, hydrogen, or from C₁₋₄ alkyl, halogenated C₁₋₄ alkyl and halogenated C₁₋₄ alkoxy, each of which is optionally deuterated;
each m is independently an integer of 0-2; and
each n is independently an integer of 0-4.

Solution 4: the compound or the pharmaceutically acceptable salt, ester, deuterated compound or stereoisomer thereof according to any of the preceding solutions, wherein:
X₁, X₂, X₃ and X₄ are each independently selected from N;
R¹, R² and R³ are each independently selected from phenyl, furyl, thienyl, pyrrolyl, thiazolyl, isothiazolyl, thiadiazolyl, oxazolyl, isoxazolyl, oxadiazole, imidazolyl, pyrazolyl, 1,2,3-triazolyl, 1,2,4-triazolyl, pyridyl, 2-pyridone, 4-pyridone, pyrimidinyl, pyridazinyl, pyrazinyl, 1,2,3-triazolyl, 1,3,5-triazinyl and 1,2,4,5 -tetrazinyl, each of which is optionally substituted by 1-3 Q₁;
R⁴ and R⁵ are each independently selected from deuterium, hydrogen, or from methyl, ethyl, propyl, isopropyl, butyl, isobutyl, sec-butyl, tert-butyl, methoxy, ethoxy, propoxy, isopropoxy, methylamino, dimethylamino, monofluoromethyl, difluoromethyl, monofluoromethoxy, difluoromethoxy, trifluoromethyl, hydroxymethyl, hydroxyethyl, hydroxypropyl, hydroxybutyl, aminomethyl, carboxymethyl, carboxyethyl and trifluoromethoxy, each of which is optionally deuterated;
each Q₁ is independently selected from deuterium, fluorine, chlorine, bromine, iodine, or from methoxy, ethoxy, propoxy, isopropoxy, methylamino, dimethylamino, monofluoromethyl, difluoromethyl, trifluoromethyl, monofluoromethoxy, difluoromethoxy, trifluoromethoxy, hydroxymethyl, hydroxyethyl, hydroxypropyl, hydroxybutyl, aminomethyl, carboxymethyl, carboxyethyl, —(L)ₘ-C₁₋₄ alkyl, or —(L)ₘ-3-6 membered cycloalkyl, each of which is optionally substituted by 1-3 substituents Q₂, and each Q₂ is independently selected from deuterium, halogen, carboxyl, hydroxyl, cyano, nitro, amino, methyl, ethyl, propyl, isopropyl, butyl, isobutyl, sec-butyl, tert-butyl, methoxy, ethoxy, propoxy, isopropoxy, hydroxymethyl, hydroxyethyl, hydroxypropyl, hydroxybutyl, carboxymethyl, carboxyethyl, methylamino, dimethylamino, monofluoromethyl, difluoromethyl, trifluoromethyl, monofluoromethoxy, difluoromethoxy and trifluoromethoxy;
each L is independently selected from —CR^{a}R^{b}—;
each R^{a} and each R^{b} are each independently selected from deuterium, hydrogen, or from methyl, ethyl, propyl, isopropyl, butyl, isobutyl, sec-butyl, tert-butyl, methoxy, ethoxy, propoxy or isopropoxy, each of which is optionally deuterated;
each m is independently selected from 0, 1 or 2; and
each n is independently 0, 1, 2 or 3.

Solution 5: the compound, the pharmaceutically acceptable salt thereof, the ester thereof, the deuterated compound or the stereoisomer thereof according to any of the preceding solutions, wherein:
R¹, R² and R³ are each independently selected from phenyl or 5-6 membered nitrogen-containing heteroaryl, each of which is optionally substituted by 1-4 Q₁.

Solution 6: the compound or the pharmaceutically acceptable salt, ester, deuterated compound or stereoisomer thereof according to any of the preceding solutions, wherein:
R¹, R² and R³ are each independently selected from phenyl, furyl, thienyl, pyrrolyl, thiazolyl, isothiazolyl, oxazolyl, isoxazolyl, imidazolyl, pyrazolyl, pyridyl, pyrimidinyl, pyridazinyl and pyrazinyl, each of which is optionally substituted by 1-3 Q₁.

Solution 7: the compound or the pharmaceutically acceptable salt, ester, deuterated compound or stereoisomer thereof according to any of the preceding solutions, wherein:
R¹, R² and R³ are each independently selected from phenyl, pyrrolyl, thiazolyl, isothiazolyl, oxazolyl, isoxazolyl, imidazolyl, pyrazolyl, pyridyl, pyrimidinyl, pyridazinyl and pyrazinyl, each of which is optionally substituted by 1-3 Q₁.

Solution 7-1: the compound or the pharmaceutically acceptable salt, ester, deuterated compound or stereoisomer thereof according to any of the preceding solutions, wherein:
R¹, R² and R³ are each independently selected from phenyl, pyrimidinyl, pyridyl, pyrazolyl, imidazolyl, pyrrolyl, pyridazinyl and pyrazinyl, each of which is optionally substituted by 1-3 Q₁.

Solution 7-2: the compound or the pharmaceutically acceptable salt, ester, deuterated compound or stereoisomer thereof according to any of the preceding solutions, wherein:

R¹ and R³ are each independently selected from

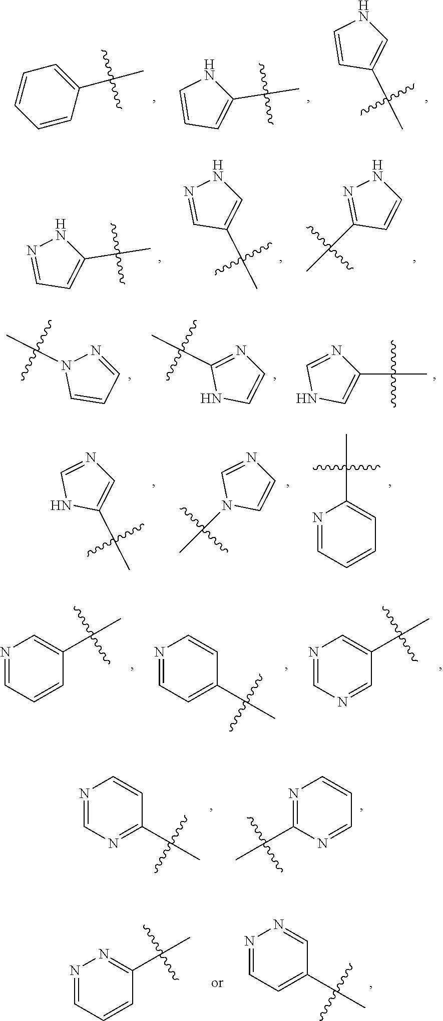

each of which is optionally substituted by 1-3 Q₁.

Solution 7-3: the compound or the pharmaceutically acceptable salt, ester, deuterated compound or stereoisomer thereof according to any of the preceding solutions, wherein:

R¹ is selected from

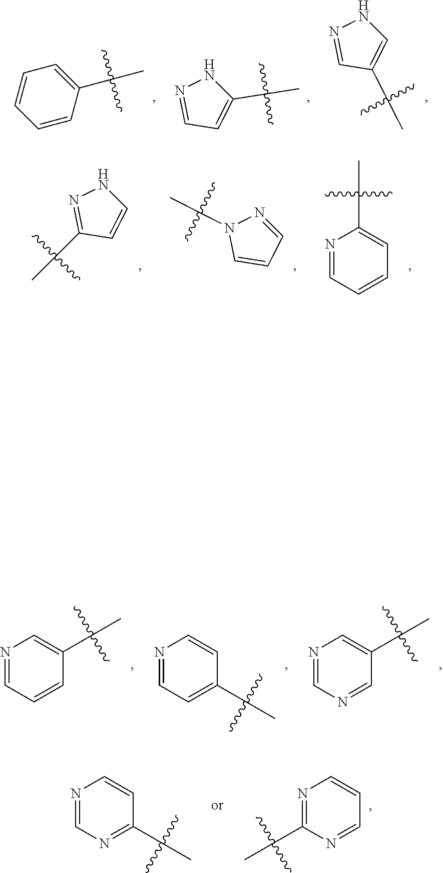

each of which is optionally substituted by 1-3 Q₁; preferably, R¹ is selected from

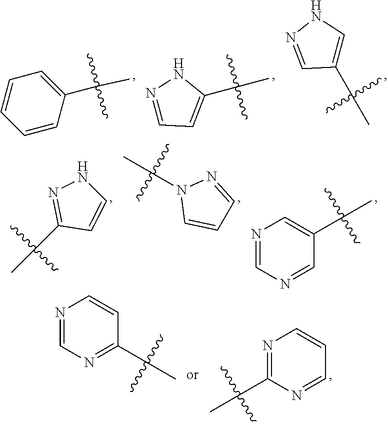

each of which is optionally substituted by 1-3 Q₁.

Solution 7-4: the compound or the pharmaceutically acceptable salt, ester, deuterated compound or stereoisomer thereof according to any of the preceding solutions, wherein:

R³ is selected from

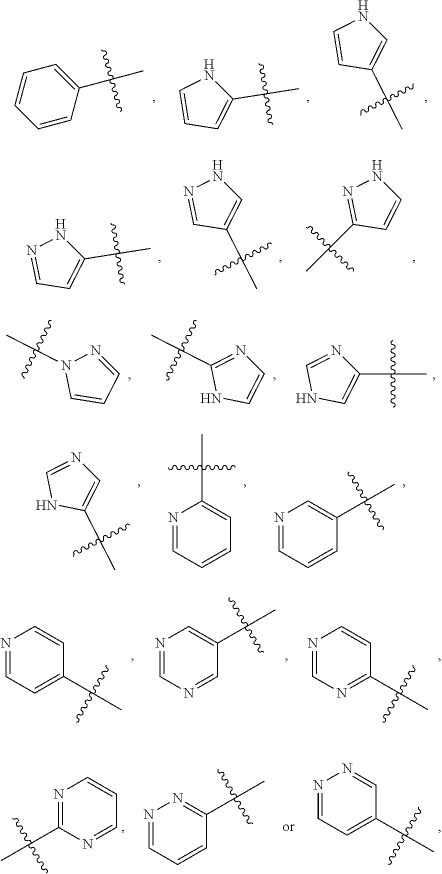

each of which is optionally substituted by 1-3 Q₁; preferably, R³ is selected from

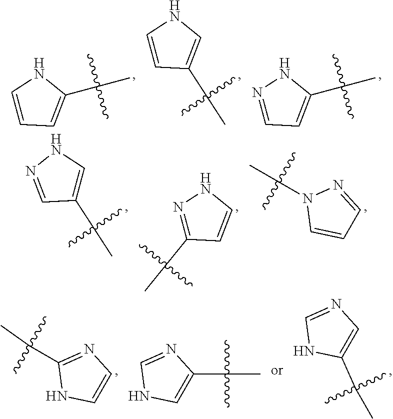

each of which is optionally substituted by 1-3 Q₁; and preferably, R³ is selected from

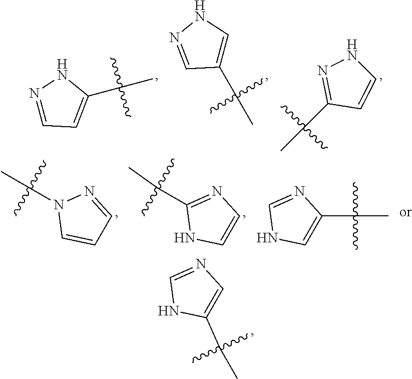

each of which is optionally substituted by 1-3 Q₁.

Solution 7-5: the compound or the pharmaceutically acceptable salt, ester, deuterated compound or stereoisomer thereof according to any of the preceding solutions, wherein:

R² is selected from phenyl or pyridyl optionally substituted by 1-3 Q₁.

Solution 8: the compound or the pharmaceutically acceptable salt, ester, deuterated compound or stereoisomer thereof according to any of the preceding solutions, has a structure of formula (II),

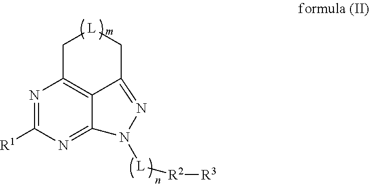

wherein, each L, each R^{a}, each R^{b}, each R^{c}, R¹, R², R³, each Q₁, each Q₂, m and n are as defined in any of the preceding solutions.

Solution 8-1: the compound or the pharmaceutically acceptable salt, ester, deuterated compound or stereoisomer thereof according to any of the preceding solutions, has a structure of formula (II-1),

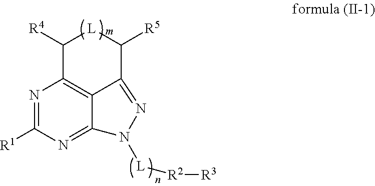

wherein, each L, each R^{a}, each R^{b}, each R^{c}, R¹, R², R³, R⁴, R⁵, each Q₁, each Q₂, m and n are as defined in any of the preceding solutions.

Solution 9: the compound or the pharmaceutically acceptable salt, ester, deuterated compound or stereoisomer thereof according to any of the preceding solutions, has a structure of general formula (III):

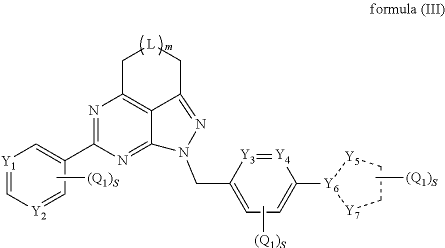

wherein:
Y₁, Y₂, Y₃, Y₄ and Y₆ are each independently selected from N, C or CR^{a};
Y₅ and Y₇ are each independently selected from N, NR^{c}, C, CR^{a} R^{b} or CR^{a};
[symbol] and - is each independently selected from a single bond or a double bond, and two adjacent bonds are incapable of being double bonds at the same time;

each s is independently selected from an integer of 0-2; and

each Q₁, m, each L, each R^{a}, each R^{b} and each R^{c} are as defined in any of the preceding solutions.

Solution 9-1: the compound or the pharmaceutically acceptable salt, ester, deuterated compound or stereoisomer thereof according to any of the preceding solutions, has a structure of general formula (III-1):

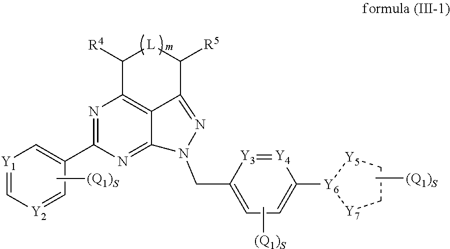

wherein:
Y₁, Y₂, Y₃, Y₄ and Y₆ are each independently selected from N, C or CR^{a};
Y₅ and Y₇ are each independently selected from N, NR^{c}, C, CR^{a}R^{b} or CR^{a};
[symbol] and - is each independently selected from a single bond or a double bond, and two adjacent bonds are incapable of being double bonds at the same time;
each s is independently selected from an integer of 0-3; and
each Q₁, m, each L, each R^{a}, each R^{b}, each R^{c}, R⁴ and R⁵ are as defined in any of the preceding solutions.

Solution 9-2: the compound or the pharmaceutically acceptable salt, ester, deuterated compound or stereoisomer thereof according to any of the preceding solutions, has a structure of general formula (III-1′):

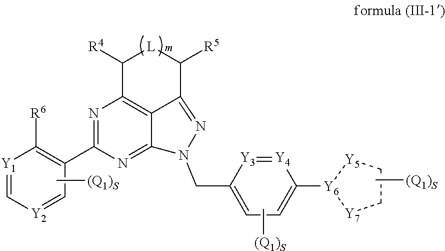

wherein, R⁶ is selected from hydrogen, or C₁₋₆ alkyl, C₁₋₆ alkoxy, halogenated C₁₋₆ alkyl and halogenated C₁₋₆ alkoxy, each of which is optionally deuterated;
Y₁, Y₂, Y₃, Y₄ and Y₆ are each independently selected from N, C or CR^{a};
Y₅ and Y₇ are each independently selected from N, NR^{c}, C, CR^{a}R^{b} or CR^{a};
[symbol] and - is each independently selected from a single bond or a double bond, and two adjacent bonds are incapable of being double bonds at the same time;
each s is independently selected from an integer of 0-3; and
each Q₁, m, each L, each R^{a}, each R^{b}, each R^{c}, R⁴ and R⁵ are as defined in any of the preceding solutions.

Solution 9-3: the compound or the pharmaceutically acceptable salt, ester, deuterated compound or stereoisomer thereof according to any of the preceding solutions, wherein R⁶ is selected from hydrogen, deuterated C₁₋₆ alkyl or deuterated C₁₋₆ alkoxy.

Solution 9-4: the compound or the pharmaceutically acceptable salt, ester, deuterated compound or stereoisomer thereof according to any of the preceding solutions, wherein:
Y₁, Y₂, Y₃, Y₄ and Y₆ are each independently selected from N, C or CH; and
Y₅ and Y₇ are each independently selected from N, NH, C, CH₂ or CH.

Solution 9-5: the compound or the pharmaceutically acceptable salt, ester, deuterated compound or stereoisomer thereof according to any of the preceding solutions, wherein:

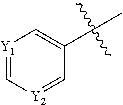

is selected from the following structures:

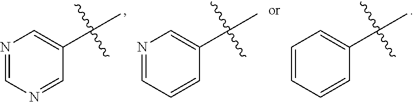 [SMILES: *c1cncnc1 ; *c1cccnc1 ; *c1ccccc1]

Solution 9-6: the compound or the pharmaceutically acceptable salt, ester, deuterated compound or stereoisomer thereof according to any of the preceding solutions, wherein:

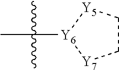

is selected from the following structures:

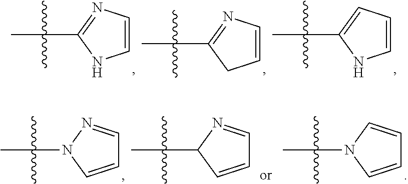

Solution 10: the compound or the pharmaceutically acceptable salt, ester, deuterated compound or stereoisomer thereof according to any of the preceding solutions, has a structure of general formula (IV):

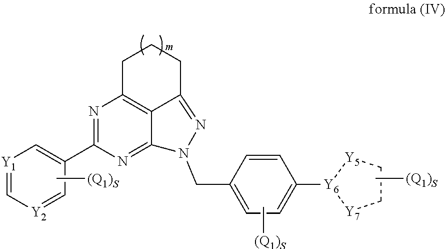

wherein:
each Q₁ and m are as defined in any of the preceding solutions;
[symbol] and - is each independently selected from a single bond or a double bond, and two adjacent bonds are incapable of being double bonds at the same time; and
Y₁, Y₂, Y₅, Y₆, Y₇ and each s are as defined in any of the preceding solutions.

Solution 10-1: the compound or the pharmaceutically acceptable salt, ester, deuterated compound or stereoisomer thereof according to any of the preceding solutions, has a structure of general formula (IV-1):

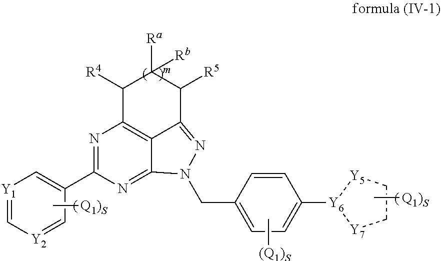

wherein:
[symbol] and - is each independently selected from a single bond or a double bond, and two adjacent bonds are incapable of being double bonds at the same time; and
each Q₁, m, R⁴, R⁵, each R^{a}, each R^{b} , Y₁, Y₂, Y₅, Y₆, Y₇ and each s are as defined in any of the preceding solutions.

Solution 10-2: the compound or the pharmaceutically acceptable salt, ester, deuterated compound or stereoisomer thereof according to any of the preceding solutions, has a structure of general formula (IV-1′):

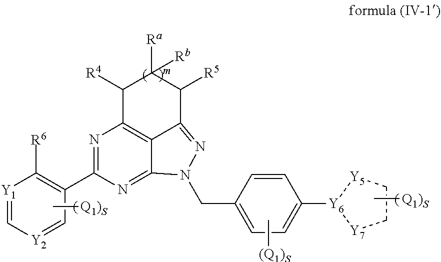

wherein, R⁶ is selected from hydrogen, or optionally deuterated C₁₋₆ alkyl, C₁₋₆ alkoxy, halogenated C₁₋₆ alkyl and halogenated C₁₋₆ alkoxy;
[symbol] and - is each independently selected from a single bond or a double bond, and two adjacent bonds are incapable of being double bonds at the same time; and
each Q₁, m, R⁴, R⁵, each R^{a}, each R^{b}, Y₁, Y₂, Y₅, Y₆, Y₇ and each s are as defined in any of the preceding solutions.

Solution 10-3: the compound or the pharmaceutically acceptable salt, ester, deuterated compound or stereoisomer thereof according to any of the preceding solutions, wherein R⁶ is selected from hydrogen, deuterated C₁₋₆ alkyl or deuterated C₁₋₆ alkoxy.

Solution 10-4: the compound or the pharmaceutically acceptable salt, ester, deuterated compound or stereoisomer thereof according to any of the preceding solutions, wherein R^{a} and R^{b} are each independently selected from hydrogen.

Solution 11: the compound or the pharmaceutically acceptable salt, ester, deuterated compound or stereoisomer thereof according to any of the preceding solutions, has a structure of general formula (V):

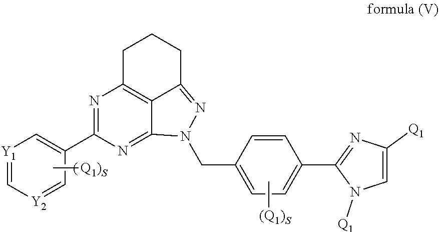

wherein:
each Q₁, Y₁, Y₂ and each s are as defined in any of the preceding solutions.

Solution 11-1: the compound or the pharmaceutically acceptable salt, ester, deuterated compound or stereoisomer thereof according to any of the preceding solutions, has a structure of general formula (V-1):

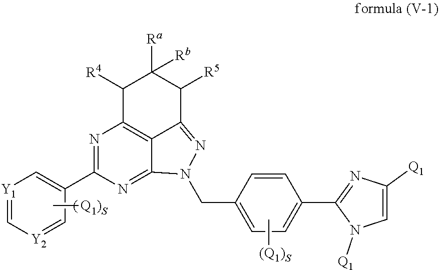

wherein:
each Q₁, R⁴, R⁵, R^{a}, R^{b}, Y₁, Y₂ and each s are as defined in any of the preceding solutions.

Solution 11-2: the compound or the pharmaceutically acceptable salt, ester, deuterated compound or stereoisomer thereof according to any of the preceding solutions, has a structure of general formula (V-1′):

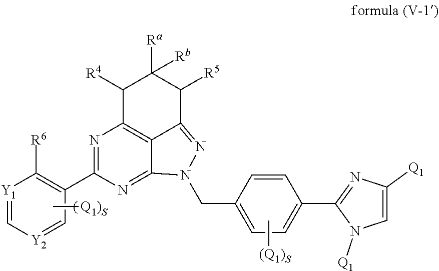

wherein, R⁶ is selected from hydrogen, or from C₁₋₆ alkyl, C₁₋₆ alkoxy, halogenated C₁₋₆ alkyl and halogenated C₁₋₆ alkoxy, each of which is optionally deuterated; and
each Q₁, R⁴, R⁵, R^{a}, R^{b}, Y₁, Y₂ and each s are as defined in any of the preceding solutions.

Solution 11-3: the compound or the pharmaceutically acceptable salt, ester, deuterated compound or stereoisomer thereof according to any of the preceding solutions, wherein R⁶ is selected from hydrogen, deuterated C₁₋₆ alkyl or deuterated C₁₋₆ alkoxy.

Solution 12: the compound or the pharmaceutically acceptable salt, ester, deuterated compound or stereoisomer thereof according to any of the preceding solutions, wherein:
each Q₁ is independently selected from deuterium, halogen, or from C₁₋₆ alkyl, C₁₋₆ alkoxy, C₁₋₆ alkylamino, di(C₁₋₆ alkyl)amino, halogenated C₁₋₆ alkyl, halogenated C₁₋₆ alkoxy, hydroxy C₁₋₆ alkyl, amino C₁₋₆ alkyl, carboxyl C₁₋₆ alkyl, —(L)ₘ-3-6 membered cycloalkyl, or —(L)ₘ-3-6 membered heterocyclyl, each of which is optionally substituted by 1-4 substituents Q₂; and each Q₂ is independently selected from deuterium, halogen, carboxyl, hydroxyl, cyano, nitro, amino, C₁₋₆ alkyl, C₁₋₆ alkoxy, halogenated C₁₋₆ alkyl and halogenated C₁₋₆ alkoxy.

Solution 12-1: the compound or the pharmaceutically acceptable salt, ester, deuterated compound or stereoisomer thereof according to any of the preceding solutions, wherein:
each Q₁ is independently selected from deuterium, halogen, or from C₁₋₆ alkyl, C₁₋₆ alkoxy, halogenated C₁₋₆ alkyl, halogenated C₁₋₆ alkoxy and 3-6 membered cycloalkyl, each of which is optionally substituted by 1-3 substituents Q₂; and each Q₂ is independently selected from deuterium, halogen, C₁₋₆ alkyl, C₁₋₆ alkoxy, halogenated C₁₋₆ alkyl and halogenated C₁₋₆ alkoxy.

Solution 13: the compound or the pharmaceutically acceptable salt, ester, deuterated compound or stereoisomer thereof according to any of the preceding solutions, wherein:
each Q₁ is independently selected from deuterium, halogen, or from C₁₋₄ alkyl, C₁₋₄ alkoxy, C₁₋₄ alkylamino, di(C₁₋₄ alkyl)amino, halogenated C₁₋₄ alkyl, halogenated C₁₋₄ alkoxy, hydroxy C₁₋₄ alkyl, amino C₁₋₄ alkyl, carboxyl C₁₋₄ alkyl and 3-6 membered cycloalkyl, each of which is optionally substituted by 1-3 substituents Q₂; and each Q₂ is independently selected from deuterium, halogen, carboxyl, hydroxyl, cyano, nitro, amino, C₁₋₄ alkyl, C₁₋₄ alkoxy, halogenated C₁₋₄ alkyl and halogenated C₁₋₄ alkoxy.

Solution 13-1: the compound or the pharmaceutically acceptable salt, ester, deuterated compound or stereoisomer thereof according to any of the preceding solutions, wherein:
each Q₁ is independently selected from deuterium, halogen, or from C₁₋₄ alkyl, C₁₋₄ alkoxy, halogenated C₁₋₄ alkyl, halogenated C₁₋₄ alkoxy and 3-6 membered cycloalkyl, each of which is optionally substituted by 1-3 substituents Q₂; and each Q₂ is independently selected from deuterium, C₁₋₄ alkyl, C₁₋₄ alkoxy, halogenated C₁₋₄ alkyl and halogenated C₁₋₄ alkoxy.

Solution 14: the compound or the pharmaceutically acceptable salt, ester, deuterated compound or stereoisomer thereof according to any of the preceding solutions, wherein:
each Q₁ is independently selected from deuterium, fluorine, chlorine, bromine, iodine, or from methyl, ethyl, propyl, isopropyl, butyl, isobutyl, sec-butyl, tert-butyl, methoxy, ethoxy, propoxy, isopropoxy, methylamino, dimethylamino, monofluoromethyl, difluoromethyl, trifluoromethyl, monofluoromethoxy, difluoromethoxy, trifluoromethoxy, hydroxymethyl, hydroxyethyl, hydroxypropyl, hydroxybutyl, aminomethyl, carboxymethyl, carboxyethyl, cyclopropyl, cyclobutyl or cyclopentyl, each of which is optionally substituted by 1-3 substituents Q₂; and each Q₂ is independently selected from deuterium, halogen, carboxyl, hydroxyl, cyano, nitro, amino, methyl, ethyl, propyl, isopropyl, butyl, isobutyl, sec-butyl, tert-butyl, methoxy, ethoxy, propoxy, isopropoxy, monofluoromethyl, difluoromethyl, trifluoromethyl, monofluoromethoxy, difluoromethoxy or trifluoromethoxy.

Solution 14-1: the compound or the pharmaceutically acceptable salt, ester, deuterated compound or stereoisomer thereof according to any of the preceding solutions, wherein:
each Q₁ is independently selected from deuterium, fluorine, chlorine, bromine, iodine, or from methyl, ethyl, propyl, isopropyl, butyl, isobutyl, sec-butyl, tert-butyl, methoxy, ethoxy, propoxy, isopropoxy, monofluoromethyl, difluoromethyl, trifluoromethyl, monofluoromethoxy, difluoromethoxy, trifluoromethoxy, cyclopropyl or cyclobutyl, cyclopentyl, each of which is optionally substituted by 1-3 substituents Q₂; and each Q₂ is independently selected from deuterium, fluorine, chlorine, bromine, iodine, methyl, ethyl, propyl, isopropyl, butyl, isobutyl, sec-butyl, tert-butyl, methoxy, ethoxy, propoxy, isopropoxy, monofluoromethyl, difluoromethyl, trifluoromethyl, monofluoromethoxy, difluoromethoxy and trifluoromethoxy.

Solution 15: the compound or the pharmaceutically acceptable salt, ester, deuterated compound or stereoisomer thereof according to any of the preceding solutions, wherein:
each L is independently selected from —CR^{a}R^{b}—; and
each R^{a} and each R^{b} are each independently selected from deuterium, hydrogen, or from C₁₋₄ alkyl, C₁₋₄ alkoxy, halogenated C₁₋₄ alkyl and halogenated C₁₋₄ alkoxy, each of which is optionally deuterated.

Solution 15-1: the compound or the pharmaceutically acceptable salt, ester, deuterated compound or stereoisomer thereof according to any of the preceding solutions, wherein:
each L is independently selected from —CR^{a}R^{b}—; and
each R^{a} and each R^{b} are each independently selected from deuterium, hydrogen, or from C₁₋₆ alkyl, C₁₋₆ alkoxy, halogenated C₁₋₆ alkyl and halogenated C₁₋₆ alkoxy, each of which is optionally deuterated.

Solution 16: the compound or the pharmaceutically acceptable salt, ester, deuterated compound or stereoisomer thereof according to any of the preceding solutions, wherein:
each L is independently selected from —CH₂—.

Solution 17: the compound or the pharmaceutically acceptable salt, ester, deuterated compound or stereoisomer thereof according to any of the preceding solutions, has a structure of general formula (VI):

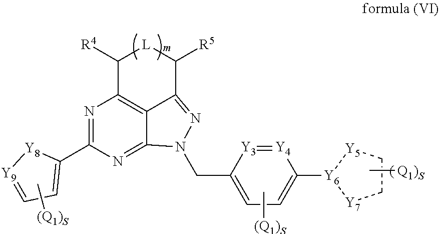

Y₈ is selected from N, NR^{c}, C, CR^{a}R^{b} or CR^{a};
Y₉ is selected from N, C or CR^{a};
[symbol] and - is each independently selected from a single bond or a double bond, and two adjacent bonds are incapable of being double bonds at the same time; and
each Q₁, each L, m, R⁴, R⁵, Y₃, Y₄, Y₅, Y₆, Y₇ and each s are as defined in any of the preceding solutions.

Solution 17-1: the compound or the pharmaceutically acceptable salt, ester, deuterated compound or stereoisomer thereof according to the preceding solution 17, wherein:
Y₈ is selected from N, NH, C, CH₂ or CH;
Y₉ is selected from N, C or CH;
[symbol] and - is each independently selected from a single bond or a double bond, and two adjacent bonds are incapable of being double bonds at the same time; and
each Q₁, each L, m, R⁴, R⁵, Y₃, Y₄, Y₅, Y₆, Y₇ and each s are as defined in any of the preceding solutions.

Solution 18: the compound or the pharmaceutically acceptable salt, the ester, deuterated compound or stereoisomer thereof according to any of the preceding solutions, has a structure of general formula (VII):

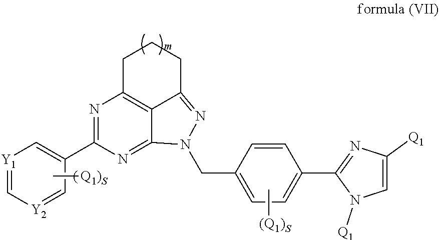

wherein, each Q₁, m, Y₁, Y₂ and each s are as defined in any of the preceding solutions.

Solution 18-1: the compound or the pharmaceutically acceptable salt, ester, deuterated compound or stereoisomer thereof according to any of the preceding solutions, has a structure of general formula (VII-1):

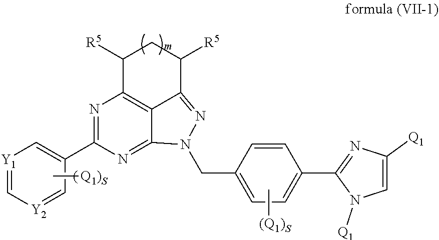

wherein, each Q₁, m, Y₁, Y₂, each s, R⁴ and R⁵ are as defined in any of the preceding solutions.

Solution 18-2: the compound or the pharmaceutically acceptable salt, ester, deuterated compound or stereoisomer thereof according to any of the preceding solutions, has a structure of general formula (VII-1′):

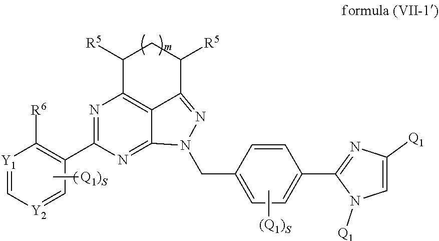

wherein, R⁶ is selected from hydrogen, or from C₁₋₆ alkyl, C₁₋₆ alkoxy, halogenated C₁₋₆ alkyl and halogenated C₁₋₆ alkoxy, each of which is optionally deuterated; and
each Q₁, m, Y₁, Y₂, each s, R⁴ and R⁵ are as defined in any of the preceding solutions.

Solution 18-3: the compound or the pharmaceutically acceptable salt, ester, deuterated compound or stereoisomer thereof according to any of the preceding solutions, wherein R⁶ is selected from hydrogen, deuterated C₁₋₆ alkyl or deuterated C₁₋₆ alkoxy.

Solution 19: the compound or the pharmaceutically acceptable salt, ester, deuterated compound or stereoisomer thereof according to any of the preceding solutions, has a structure of general formula (VIII):

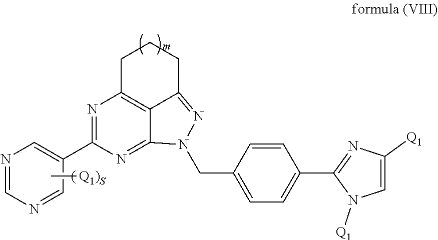

wherein, each Q₁, m and each s are as defined in any of the preceding solutions.

Solution 19-1: the compound or the pharmaceutically acceptable salt, ester, deuterated compound or stereoisomer thereof according to any of the preceding solutions, has a structure of general formula (VIII-1):

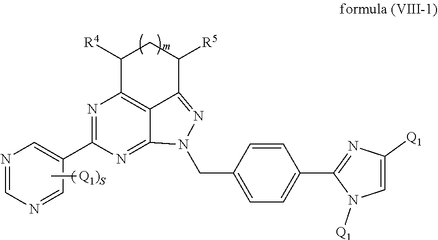

wherein, each Q₁, m, R⁴, R⁵ and each s are as defined in any of the preceding solutions.

Solution 19-2: the compound or the pharmaceutically acceptable salt, ester, deuterated compound or stereoisomer thereof according to any of the preceding solutions, has a structure of general formula (VIII-1′):

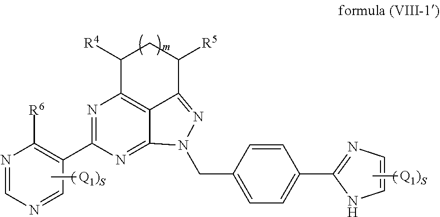

wherein, R⁶ is selected from hydrogen, or from C₁₋₆ alkyl, C₁₋₆ alkoxy, halogenated C₁₋₆ alkyl and halogenated C₁₋₆ alkoxy, each of which is optionally deuterated; and
each Q₁, m, R⁴, R⁵ and each s are as defined in any of the preceding solutions.

Solution 19-3: the compound or the pharmaceutically acceptable salt, ester, deuterated compound or stereoisomer thereof according to any of the preceding solutions, wherein R⁶ is selected from hydrogen, deuterated C₁₋₆ alkyl or deuterated C₁₋₆ alkoxy.

Solution 20: the compound or the pharmaceutically acceptable salt, ester, deuterated compound or stereoisomer thereof according to any of the preceding solutions, has a structure of general formula (IX):

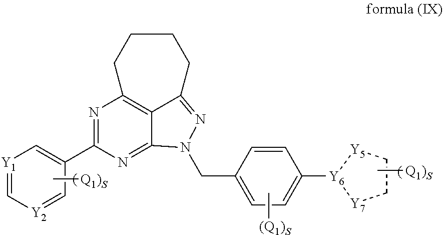

[symbol] and - is each independently selected from a single bond or a double bond, and two adjacent bonds are incapable of being double bonds at the same time; and
each Q₁, Y₁, Y₂, Y₅, Y₆, Y₇ and each s are as defined in any of the preceding solutions.

Solution 20-1: the compound or the pharmaceutically acceptable salt, ester, deuterated compound or stereoisomer thereof according to any of the preceding solutions, has a structure of general formula (IX-1):

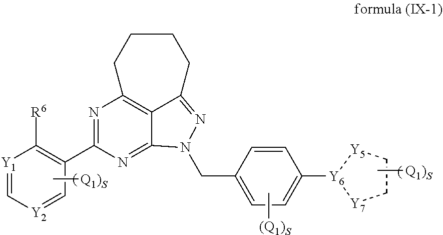

wherein, R⁶ is selected from hydrogen, or optionally deuterated C₁₋₆ alkyl, C₁₋₆ alkoxy, halogenated C₁₋₆ alkyl and halogenated C₁₋₆ alkoxy;
[symbol] and - is each independently selected from a single bond or a double bond, and two adjacent bonds are incapable of being double bonds at the same time; and
each Q₁, Y₁, Y₂, Y₅, Y₆, Y₇ and each s are as defined in any of the preceding solutions.

Solution 20-2: the compound or the pharmaceutically acceptable salt, ester, deuterated compound or stereoisomer thereof according to any of the preceding solutions, where R⁶ is selected from hydrogen, or optionally deuterated C₁₋₆ alkyl or deuterated C₁₋₆ alkoxy.

Solution 21: the compound or the pharmaceutically acceptable salt, ester, deuterated compound or stereoisomer thereof according to any of the preceding solutions, where:
R⁴, R⁵, each R^{a}, each R^{b} and each R^{c} are each independently selected from hydrogen.

Solution 22: the compound or the pharmaceutically acceptable salt, ester, deuterated compound or stereoisomer thereof according to any of the preceding solutions, has a structure of general formula (II-2):

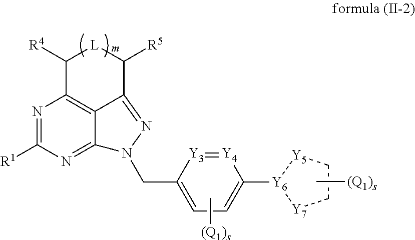

wherein, Y₃, Y₄ and Y₆ are each independently selected from N, C or CR^{a};
Y₅ and Y₇ are each independently selected from N, NR^{c}, C, CR^{a}R^{b} or CR^{a};
[symbol] and - is each independently selected from a single bond or a double bond, and two adjacent bonds are incapable of being double bonds at the same time;
each s is independently selected from an integer of 0-2; and
each Q₁, each Q₂, each L, m, R¹, R⁴, R⁵, each R^{a}, each R^{b} and each R^{c} are as defined in any of the preceding solutions.

Solution 23: the compound or the pharmaceutically acceptable salt, ester, deuterated compound or stereoisomer thereof according to any of the preceding solutions, has a structure of general formula (II-3):

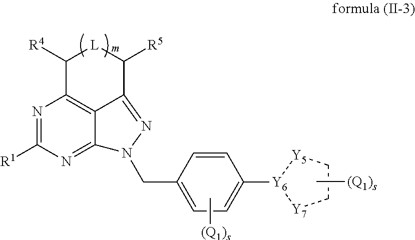

each Q₁, each Q₂, each s, each L, m, R¹, R⁴, R⁵, Y₅, Y₆, Y₇, each R^{a}, each R^{b} and each R^{c} are as defined in any of the preceding solutions.

Solution 24: the compound or the pharmaceutically acceptable salt, ester, deuterated compound or stereoisomer thereof according to any of the preceding solutions, has a structure of general formula (II-3):

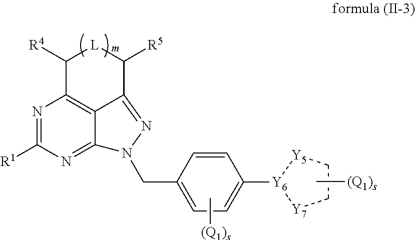

R¹ is selected from phenyl or 5-6 membered nitrogen-containing heteroaryl, each of which is optionally substituted by 1-3 Q₁; preferably, R¹ is selected from

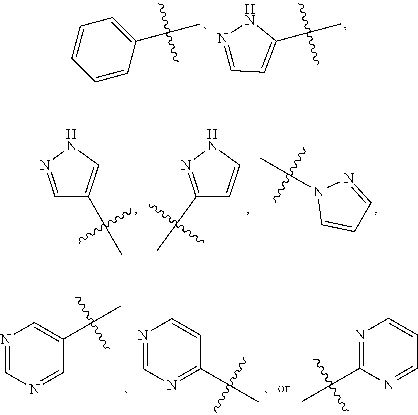

optionally substituted by 1-3 Q₁;
R⁴ and R⁵ are each independently selected from deuterium, hydrogen, or from C₁₋₆ alkyl, C₁₋₆ alkoxy, halogenated C₁₋₆ alkyl or halogenated C₁₋₆ alkoxy, each of which is optionally deuterated;
each L is independently selected from —CR^{a}R^{b}—;
each R^{a} and each R^{b} are each independently selected from deuterium, hydrogen, or from C₁₋₆ alkyl, C₁₋₆ alkoxy, halogenated C₁₋₆ alkyl or halogenated C₁₋₆ alkoxy, each of which is optionally deuterated;
each Q₁ is independently selected from deuterium, halogen, or from C₁₋₆ alkyl, C₁₋₆ alkoxy, halogenated C₁₋₆ alkyl, halogenated C₁₋₆ alkoxy or 3-6 membered cycloalkyl, each of which is optionally substituted by 1-3 substituents Q₂; and each Q₂ is independently selected from deuterium, halogen, C₁₋₆ alkyl, C₁₋₆ alkoxy, halogenated C₁₋₆ alkyl and halogenated C₁₋₆ alkoxy;

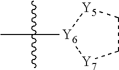

is selected from the following structure:

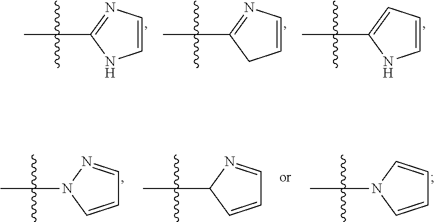 [SMILES: *c1ncc[nH]1 ; *C1=NC=CC1 ; *c1ccc[nH]1 ; *n1cccn1 ; *C1C=CC=N1 ; *n1cccc1]

m is an integer of 1 or 2; and
each s is independently an integer of 0, 1 and 2.

Solution 25: the compound or the pharmaceutically acceptable salt, ester, deuterated compound or stereoisomer thereof according to any of the preceding solutions, has a structure of general formula (II-4):

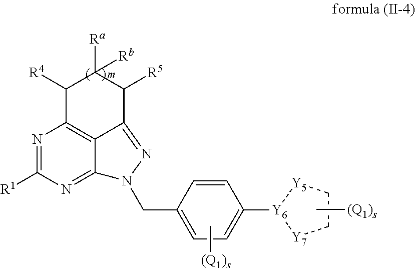

each Q₁, each Q₂, each s, m, R¹, R⁴, R⁵, Y₅, Y₆, Y₇, each R^{a}, each R^{b} and each R^{c} are as defined in any of the preceding solutions.

Solution 26: the compound or the pharmaceutically acceptable salt, ester, deuterated compound or stereoisomer thereof according to any of the preceding solutions, has a structure of general formula (II-4):

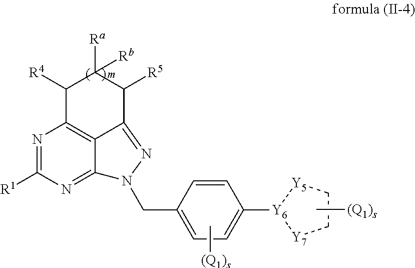

wherein, R¹ is selected from phenyl or 5-6 membered nitrogen-containing heteroaryl, each of which is optionally substituted by 1-3 Q₁; preferably, R¹ is selected from

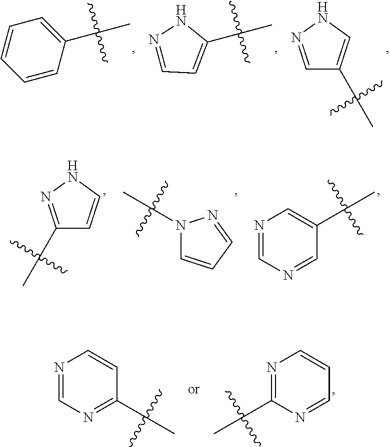

each of which is optionally substituted by 1-3 Q₁;
R⁴ and R⁵ are each independently selected from deuterium, hydrogen, or optionally deuterated C₁₋₆ alkyl, C₁₋₆ alkoxy, halogenated C₁₋₆ alkyl and halogenated C₁₋₆ alkoxy;
each Q₁ is independently selected from deuterium, halogen, or from C₁₋₆ alkyl, C₁₋₆ alkoxy, halogenated C₁₋₆ alkyl, halogenated C₁₋₆ alkoxy and 3-6 membered cycloalkyl, each of which is optionally substituted by 1-3 substituents Q₂; and each Q₂ is independently selected from deuterium, halogen, C₁₋₆ alkyl, C₁₋₆ alkoxy, halogenated C₁₋₆ alkyl and halogenated C₁₋₆ alkoxy;

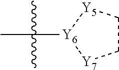

is selected from the following structure:

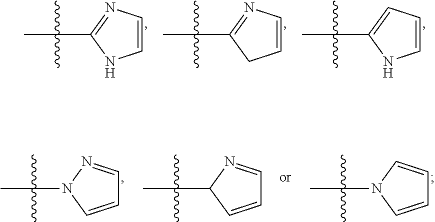 [SMILES: *c1ncc[nH]1 ; *C1=NC=CC1 ; *c1ccc[nH]1 ; *n1cccn1 ; *C1C=CC=N1 ; *n1cccc1]

m is an integer of 1 or 2; and
each s is independently an integer of 0, 1 and 2.

Solution 27: the compound or the pharmaceutically acceptable salt, ester, deuterated compound or stereoisomer thereof according to any of the preceding solutions, has a structure of general formula (II-5):

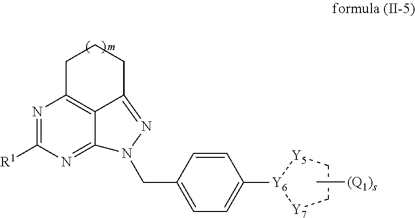

each Q₁, each Q₂, s, m, R¹, Y₅, Y₆, Y₇, each R^{a}, each R^{b} and each R^{c} are as defined in any of the preceding solutions.

Solution 28: the compound or the pharmaceutically acceptable salt, ester, deuterated compound or stereoisomer thereof according to any of the preceding solutions, has a structure of general formula (II-5):

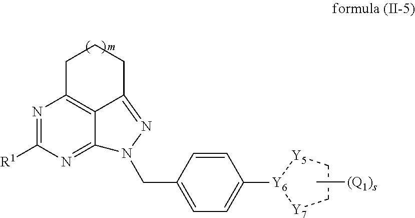

R¹ is selected from phenyl or 5-6 membered nitrogen-containing heteroaryl optionally substituted by 1-3 Q₁; preferably, R¹ is selected from

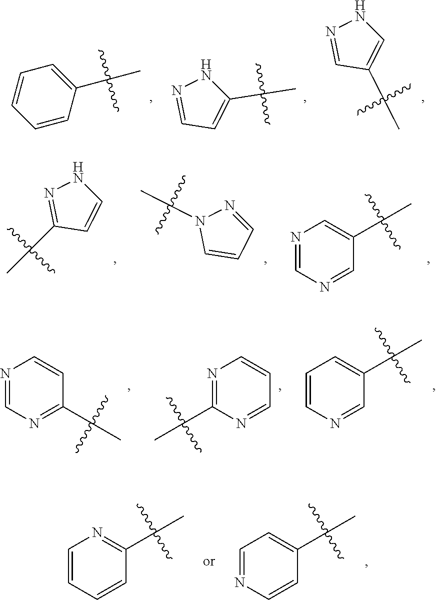

each of which is optionally substituted by 1-3 Q₁;
each Q₁ is independently selected from deuterium, halogen, or from C₁₋₆ alkyl, C₁₋₆ alkoxy, halogenated C₁₋₆ alkyl, halogenated C₁₋₆ alkoxy or 3-6 membered cycloalkyl, each of which is optionally substituted by 1-3 substituents Q₂; and each Q₂ is independently selected from deuterium, C₁₋₆ alkyl, C₁₋₆ alkoxy, halogenated C₁₋₆ alkyl and halogenated C₁₋₆ alkoxy;

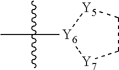

is selected from the following structure:

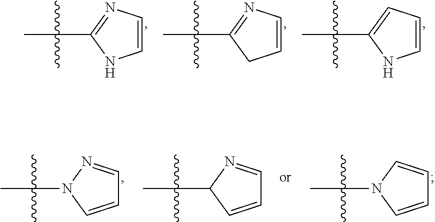 [SMILES: *c1ncc[nH]1 ; *C1=NC=CC1 ; *c1ccc[nH]1 ; *n1cccn1 ; *C1C=CC=N1 ; *n1cccc1]

m is an integer of 1 or 2; and
each s is independently an integer of 0, 1 or 2.

Solution 29: the compound or the pharmaceutically acceptable salt, ester, deuterated compound or stereoisomer thereof according to any of the preceding solutions, has a structure of general formula (IV-1′):

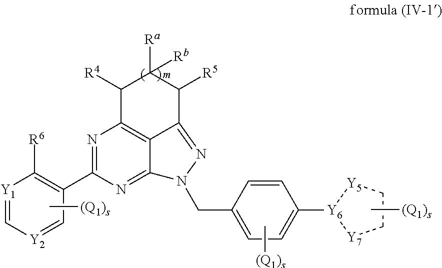

wherein, Y₁ and Y₂ are each independently selected from N, C or CH;
Y₅ and Y₇ are each independently selected from NH, N, C, CH or CH₂;
Y₆ is selected from N, CH or C;
R⁴ and R⁵ are each independently selected from deuterium, hydrogen, cyano, halogen, or C₁₋₆ alkyl, C₁₋₆ alkoxy, C₁₋₆ alkylamino, di(C₁₋₆ alkyl)amino, halogenated C₁₋₆ alkyl, hydroxy C₁₋₆ alkyl, amino C₁₋₆ alkyl, carboxyl C₁₋₆ alkyl or halogenated C₁₋₆ alkoxy, each of which is optionally deuterated;
R⁶ is selected from hydrogen and deuterated C₁₋₆ alkoxy;
each Q₁ is independently selected from deuterium, halogen, or from C₁₋₆ alkyl, C₁₋₆ alkoxy, C₁₋₆ alkylamino, di(C₁₋₆ alkyl)amino, halogenated C₁₋₆ alkyl, halogenated C₁₋₆ alkoxy, hydroxy C₁₋₆ alkyl, amino C₁₋₆ alkyl, carboxyl C₁₋₆ alkyl, —(L)ₘ-3-6 membered cycloalkyl or —(L)ₘ-3-6 membered heterocyclyl, each of which is optionally substituted by 1-3 substituents Q₂;
R^{a} and R^{b} are each independently selected from hydrogen;
each Q₂ is independently selected from deuterium, hydrogen, C₁₋₆ alkyl, C₁₋₆ alkoxy, halogenated C₁₋₆ alkyl and halogenated C₁₋₆ alkoxy;
-- and - is each independently selected from a single bond or a double bond, and two adjacent bonds are incapable of being double bonds at the same time; and
each m and each s are independently 0, 1 or 2.

Solution 30: the compound or the pharmaceutically acceptable salt, ester, deuterated compound or stereoisomer thereof according to any of the preceding solutions, has a structure of general formula (VIII-1′):

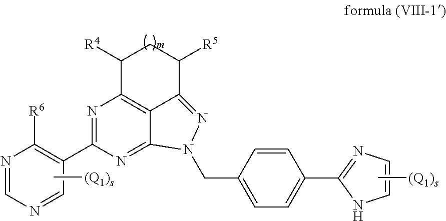

each Q₁ is independently selected from deuterium, halogen, or from C₁₋₄ alkyl, C₁₋₄ alkoxy, C₁₋₄ alkylamino, di(C₁₋₄ alkyl)amino, halogenated C₁₋₄ alkyl, halogenated C₁₋₄ alkoxy, hydroxy C₁₋₄ alkyl, amino C₁₋₄ alkyl, carboxyl C₁₋₄ alkyl or 3-6 membered cycloalkyl, each of which is optionally substituted by 1-3 substituents Q₂;
each Q₂ is independently selected from deuterium, hydrogen, C₁₋₄ alkyl, C₁₋₄ alkoxy, halogenated C₁₋₄ alkyl or halogenated C₁₋₄ alkoxy;
R⁴ and R⁵ are each independently selected from deuterium, hydrogen, cyano, halogen, or from C₁₋₆ alkyl, C₁₋₆ alkoxy, halogenated C₁₋₆ alkyl or halogenated C₁₋₆ alkoxy, each of which is optionally deuterated;
R⁶ is selected from hydrogen and deuterated C₁₋₄ alkoxy;
m is 1 or 2; and
each s is independently 0, 1 or 2.

Solution 31: the compound or the pharmaceutically acceptable salt, ester, deuterated compound or stereoisomer thereof according to any of the preceding solutions, wherein R⁶ is selected from hydrogen, methyl, ethyl, propyl, isopropyl, butyl, isobutyl, sec-butyl, tert-butyl, methoxy, ethoxy, propoxy, isopropoxy, monofluoromethyl, difluoromethyl, trifluoromethyl, monofluoromethoxy, difluoromethoxy, trifluoromethoxy, deuterated methyl, deuterated ethyl, deuterated propyl, deuterated isopropyl, deuterated butyl, deuterated isobutyl, deuterated sec-butyl, deuterated tert-butyl, deuterated methoxy, deuterated ethoxy, deuterated propoxy or deuterated isopropoxy, and a number of deuteration is 1, 2, 3 or 4.

Solution 32: the compound or the pharmaceutically acceptable salt, ester, deuterated compound or stereoisomer thereof according to any of the preceding solutions, wherein:

R⁶ is selected from hydrogen, deuterated methoxy, deuterated ethoxy, deuterated propoxy or deuterated isopropoxy, a number of deuteration is 1, 2 or 3, and trideuterated methoxy is preferred.

The selection of any substituent in any embodiment of the present disclosure may be combined with each other, and the combined new technical solution is encompassed within the protection scope of the present disclosure.

In some embodiments of the present disclosure, the structure of the compound of general formula (I) or the pharmaceutically acceptable salt, ester, deuterated compound or stereoisomer thereof is shown in Table 1:

TABLE 1

| No. | Structure |
|---|---|
| Compound 1 | 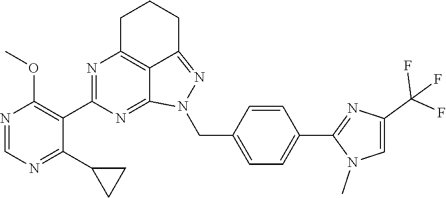 [SMILES: COc1ncnc(C2CC2)c1-c1nc2c3c(nn(Cc4ccc(-c5nc(C(F)(F)F)cn5C)cc4)c3n1)CCC2] |
| Compound 2 | 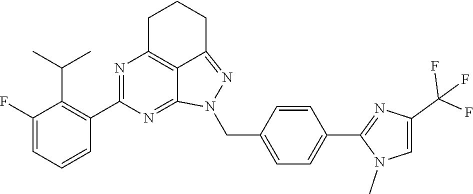 [SMILES: CC(C)c1c(F)cccc1-c1nc2c3c(nn(Cc4ccc(-c5nc(C(F)(F)F)cn5C)cc4)c3n1)CCC2] |
| Compound 3 | 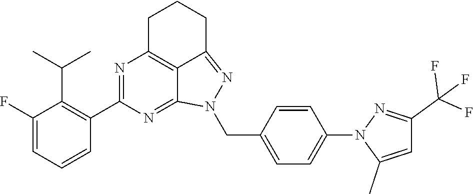 [SMILES: Cc1cc(C(F)(F)F)nn1-c1ccc(Cn2nc3c4c(nc(-c5cccc(F)c5C(C)C)nc42)CCC3)cc1] |
| Compound 4 | 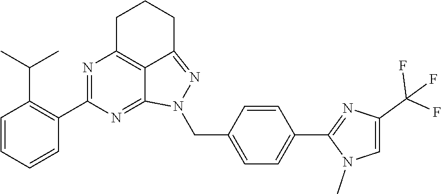 [SMILES: CC(C)c1ccccc1-c1nc2c3c(nn(Cc4ccc(-c5nc(C(F)(F)F)cn5C)cc4)c3n1)CCC2] |
| Compound 5 | 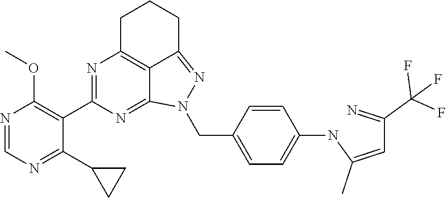 [SMILES: COc1ncnc(C2CC2)c1-c1nc2c3c(nn(Cc4ccc(N/C(C)=C\C(=N)C(F)(F)F)cc4)c3n1)CCC2] |
| Compound 6 | 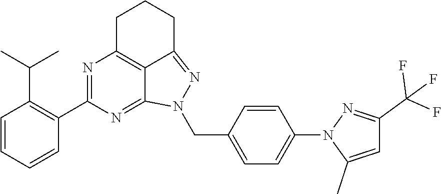 [SMILES: Cc1cc(C(F)(F)F)nn1-c1ccc(Cn2nc3c4c(nc(-c5ccccc5C(C)C)nc42)CCC3)cc1] |
| Compound 7 | 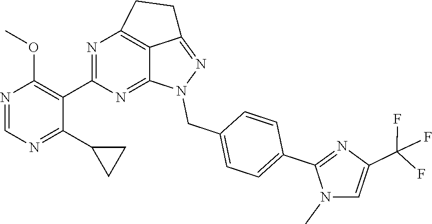 [SMILES: COc1ncnc(C2CC2)c1-c1nc2c3c(nn(Cc4ccc(-c5nc(C(F)(F)F)cn5C)cc4)c3n1)CC2] |
| Compound 8 | 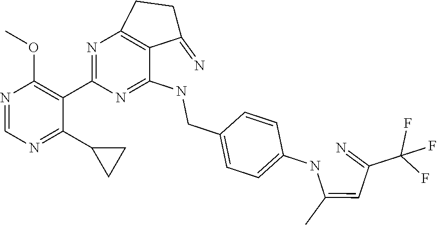 [SMILES: COc1ncnc(C2CC2)c1-c1nc2c(c(NCc3ccc(N/C(C)=C\C(=N)C(F)(F)F)cc3)n1)C(=N)CC2] |
| Compound 9 | 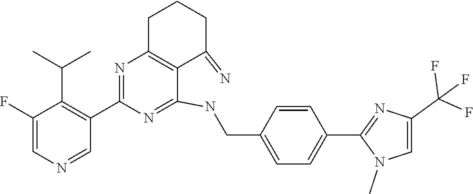 [SMILES: CC(C)c1c(F)cncc1-c1nc2c(c(NCc3ccc(-c4nc(C(F)(F)F)cn4C)cc3)n1)C(=N)CCC2] |
| Compound 10 | 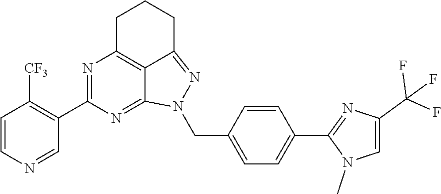 [SMILES: Cn1cc(C(F)(F)F)nc1-c1ccc(Cn2nc3c4c(nc(-c5cnccc5C(F)(F)F)nc42)CCC3)cc1] |
| Compound 11 | 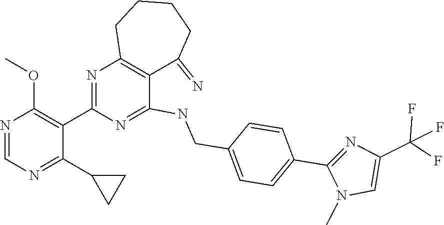 [SMILES: COc1ncnc(C2CC2)c1-c1nc2c(c(NCc3ccc(-c4nc(C(F)(F)F)cn4C)cc3)n1)C(=N)CCCC2] |
| Compound 12 | 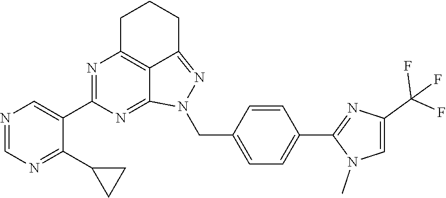 [SMILES: Cn1cc(C(F)(F)F)nc1-c1ccc(Cn2nc3c4c(nc(-c5cncnc5C5CC5)nc42)CCC3)cc1] |
| Compound 13 | 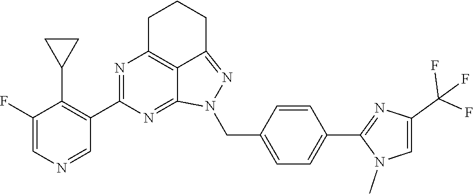 [SMILES: Cn1cc(C(F)(F)F)nc1-c1ccc(Cn2nc3c4c(nc(-c5cncc(F)c5C5CC5)nc42)CCC3)cc1] |
| Compound 14 | 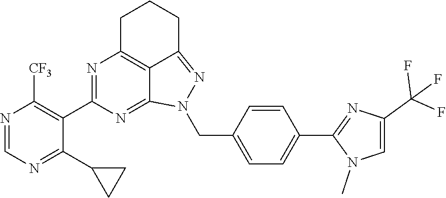 [SMILES: Cn1cc(C(F)(F)F)nc1-c1ccc(Cn2nc3c4c(nc(-c5c(C6CC6)ncnc5C(F)(F)F)nc42)CCC3)cc1] |
| Compound 15 | 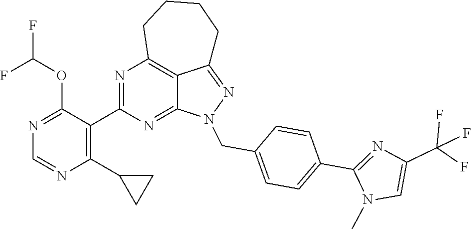 [SMILES: Cn1cc(C(F)(F)F)nc1-c1ccc(Cn2nc3c4c(nc(-c5c(OC(F)F)ncnc5C5CC5)nc42)CCCC3)cc1] |
| Compound 16 | 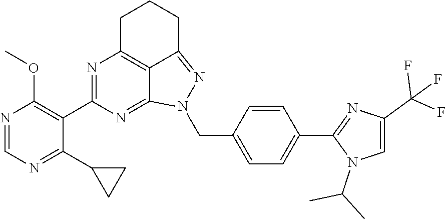 [SMILES: COc1ncnc(C2CC2)c1-c1nc2c3c(nn(Cc4ccc(-c5nc(C(F)(F)F)cn5C(C)C)cc4)c3n1)CCC2] |
| Compound 17 | 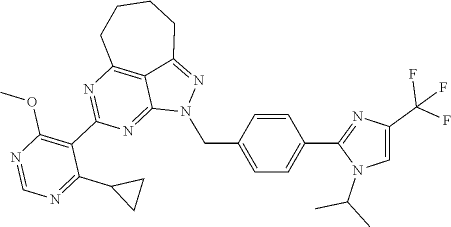 [SMILES: COc1ncnc(C2CC2)c1-c1nc2c3c(nn(Cc4ccc(-c5nc(C(F)(F)F)cn5C(C)C)cc4)c3n1)CCCC2] |
| Compound 18 | 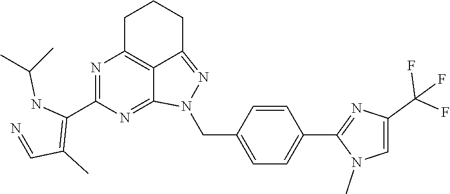 [SMILES: C/C(C=N)=C(/NC(C)C)c1nc2c3c(nn(Cc4ccc(-c5nc(C(F)(F)F)cn5C)cc4)c3n1)CCC2] |
| Compound 19 | 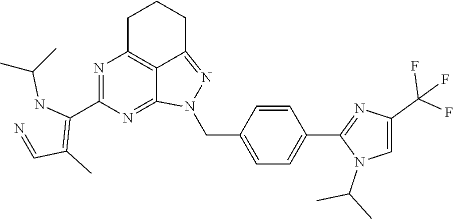 [SMILES: C/C(C=N)=C(/NC(C)C)c1nc2c3c(nn(Cc4ccc(-c5nc(C(F)(F)F)cn5C(C)C)cc4)c3n1)CCC2] |
| Compound 20 | 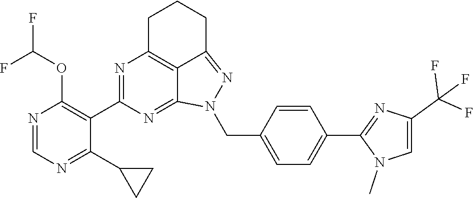 [SMILES: Cn1cc(C(F)(F)F)nc1-c1ccc(Cn2nc3c4c(nc(-c5c(OC(F)F)ncnc5C5CC5)nc42)CCC3)cc1] |
| Compound 1-1 | 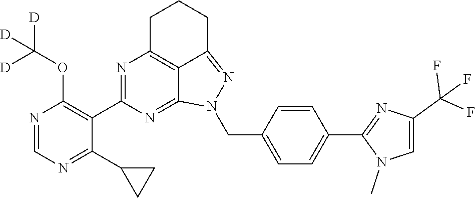 [SMILES: [2H]C([2H])([2H])Oc1ncnc(C2CC2)c1-c1nc2c3c(nn(Cc4ccc(-c5nc(C(F)(F)F)cn5C)cc4)c3n1)CCC2] |
| Compound 5-1 | 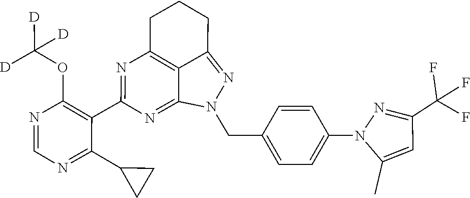 [SMILES: [2H]C([2H])([2H])Oc1ncnc(C2CC2)c1-c1nc2c3c(nn(Cc4ccc(-n5nc(C(F)(F)F)cc5C)cc4)c3n1)CCC2] |
| Compound 7-1 | 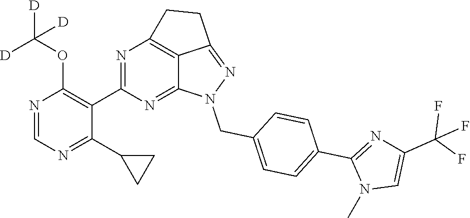 [SMILES: [2H]C([2H])([2H])Oc1ncnc(C2CC2)c1-c1nc2c3c(nn(Cc4ccc(-c5nc(C(F)(F)F)cn5C)cc4)c3n1)CC2] |
| Compound 8-1 | 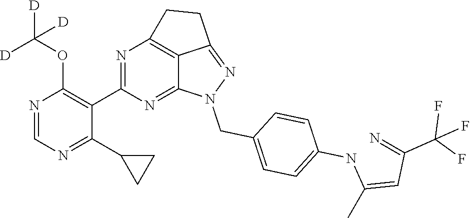 [SMILES: [2H]C([2H])([2H])Oc1ncnc(C2CC2)c1-c1nc2c3c(nn(Cc4ccc(N/C(C)=C\C(=N)C(F)(F)F)cc4)c3n1)CC2] |
| Compound 11-1 | 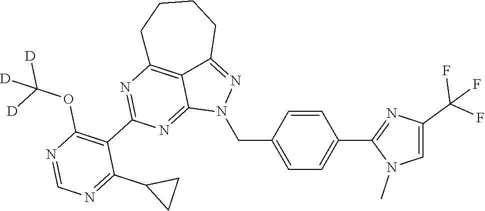 [SMILES: [2H]C([2H])([2H])Oc1ncnc(C2CC2)c1-c1nc2c3c(nn(Cc4ccc(-c5nc(C(F)(F)F)cn5C)cc4)c3n1)CCCC2] |
| Compound 16-1 | 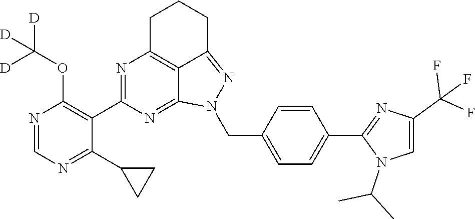 [SMILES: [2H]C([2H])([2H])Oc1ncnc(C2CC2)c1-c1nc2c3c(nn(Cc4ccc(-c5nc(C(F)(F)F)cn5C(C)C)cc4)c3n1)CCC2] |
| Compound 17-1 | 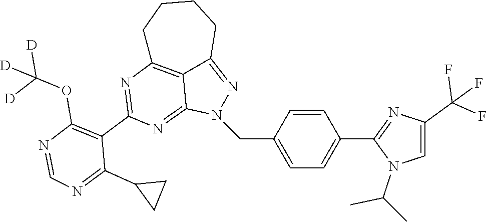 [SMILES: [2H]C([2H])([2H])Oc1ncnc(C2CC2)c1-c1nc2c3c(nn(Cc4ccc(-c5nc(C(F)(F)F)cn5C(C)C)cc4)c3n1)CCCC2] |

The “pharmaceutically acceptable salt” according to the present disclosure refers to an addition salt of a pharmaceutically acceptable acid and a base, such as a metal salt, an ammonium salt, a salt formed with an organic acid, a salt formed with an organic base, a salt formed with an inorganic acid, a salt formed with an acidic amino acid or a basic amino acid, etc.

The “ester” according to the present disclosure refers to a pharmaceutically acceptable ester, in particular to the following ester which is hydrolyzed in vivo and includes an ester of a compound or a salt thereof that is easily decomposed in a human body to leave a parent compound (the compound of general formula (I)) or a salt thereof. The “ester” according to the present disclosure, for example, may be selected from the following groups: (1) a carboxylic ester obtained by esterification with a carboxylic acid compound, where a non-carbonyl part of the carboxylic acid compound is selected from, for example, C₁₋₂₀ linear or branched alkyl, C₁₋₁₂ linear or branched alkyl, C₁₋₈ linear or branched alkyl, C₁₋₆ linear or branched alkyl (such as methyl, ethyl, n-propyl, tert-butyl or n-butyl), C₁₋₆ alkoxy C₁₋₆ alkyl (such as methoxymethyl) and C₆₋₁₀ aryl C₁₋₆ alkyl (such as benzyl), C₆₋₁₀ aryloxy C₁₋₆ alkyl (such as phenoxymethyl), and C₆₋₁₀ aryl (such as phenyl, optionally substituted by, for example, halogen, C₁₋₄ alkyl or C₁₋₄ alkoxy or amino); (2) a sulfonic acid ester, such as alkylsulfonyl or aralkylsulfonyl (such as methylsulfonyl); (3) an amino acid ester (such as L-valinyl or L-isoleucyl); (4) mono,di-or triphosphate, and the like; and (5) an ester obtained by esterification with an alcohol compound, where a non-hydroxyl part of the alcohol compound is selected from, for example, C₁₋₂₀ linear or branched alkyl, C₁₋₁₂ linear or branched alkyl, C₁₋₈ linear or branched alkyl, C₁₋₆ linear or branched alkyl (such as methyl, ethyl, n-propyl, tert-butyl or n-butyl), C₁₋₆ alkoxy C₁₋₆ alkyl (such as methoxymethyl) and C₆₋₁₀ aryl C₁₋₆ alkyl (such as benzyl), C₆₋₁₀ aryloxy C₁₋₆ alkyl (such as phenoxymethyl), and C₆₋₁₀ aryl (such as phenyl, optionally substituted by, for example, halogen, C₁₋₄ alkyl or C₁₋₄ alkoxy or amino).

The “stereoisomer” of the compound of general formula (I) in the present disclosure refers to an enantiomer generated when the compound of formula (I) has an asymmetric carbon atom; a cis and trans isomer generated when the compound has a carbon-carbon double bond or a ring structure; and a tautomer generated when the compound has ketone or oxime. In some embodiments of the present disclosure, the stereoisomer includes, but is not limited to, an enantiomer, a diastereomer, a racemate, a cis and trans isomer, a tautomer, a geometric isomer, an epimer and a mixture thereof.

The present disclosure also provides a pharmaceutical composition including the compound of general formula (I) or the pharmaceutically acceptable salt, ester, deuterated compound or stereoisomer thereof, and one or more second therapeutic active agents, and optionally, the pharmaceutical composition further includes one or more pharmaceutically acceptable carriers and/or diluents.

The present disclosure also provides a pharmaceutical formulation including the compound of general formula (I) or the pharmaceutically acceptable salt, ester, deuterated compound or stereoisomer thereof, and one or more pharmaceutically acceptable carriers and/or diluents. The pharmaceutical formulation is any dosage form that is clinically or pharmaceutically acceptable.

In some embodiments of the present disclosure, the pharmaceutical formulation described above may be administered to a patient or subject in need of such treatment in oral, parenteral, rectal, or pulmonary administration. When used for oral administration, the pharmaceutical composition may be made into an oral formulation, for example, may be made into a conventional oral solid formulation, such as a tablet, a capsule, a pill, a granule and the like; may also be made into an oral liquid formulation, such as an oral solution, an oral suspension, a syrup, and the like. When the pharmaceutical composition is made into an oral formulation, an appropriate filler, binder, disintegrant, lubricant, and the like, may be added. When used for parenteral administration, the above pharmaceutical formulation may also be made into an injection preparation, including an injection, a sterile powder for injection and a concentrated solution for injection. When the pharmaceutical formulation is made into an injection preparation, the preparation may be produced by a conventional method in the existing pharmaceutical field. When preparing the injection preparation, no additives need to be added, or appropriate additives may be added according to the properties of the drug. When used for rectal administration, the pharmaceutical composition may be made into a suppository, and the like. When used for pulmonary administration, the pharmaceutical composition may be made into an inhalant or a spray, and the like.

The pharmaceutically acceptable carriers and/or diluents that may be used in the pharmaceutical composition or the pharmaceutical formulation of the present disclosure may be any conventional carrier and/or diluent in the field of pharmaceutical formulations, and the selection of specific carrier and/or diluent will depend on the mode of administration or the type and state of diseases used for treating specific patients. Preparation methods of suitable pharmaceutical compositions for specific modes of administration are completely within the knowledge of those skilled in the pharmaceutical field. For example, the pharmaceutical carriers and/or diluents may include solvents, diluents, dispersants, suspending agents, surfactants, isotonic agents, thickeners, emulsifiers, binders, lubricants, stabilizers, hydration agents, emulsification accelerators, buffers, absorbents, colorants, ion exchangers, release agents, coating agents, correctants, antioxidants and the like, which are common in the pharmaceutical field. When necessary, flavoring agents, preservatives, sweeteners, and the like, may also be added to the pharmaceutical composition.

The present disclosure also provides a use of the compound of general formula (I), the pharmaceutically acceptable salt, ester, deuterated compound or stereoisomer thereof, the above pharmaceutical formulation or the above pharmaceutical composition in the manufacture of a medicament for treating and/or preventing a disease mediated by USP1 and a disease related thereto. The disease mediated by USP1 and the disease related thereto are selected from cancer or benign tumor.

The present disclosure also provides a use of the compound of general formula (I), the pharmaceutically acceptable salt, ester, deuterated compound or stereoisomer thereof, the above pharmaceutical formulation or the above pharmaceutical composition for treating and/or preventing a disease mediated by USP1 and a disease related thereto. The disease mediated by USP1 and the disease related thereto are selected from cancer or benign tumor.

The present disclosure also provides a method for treating a disease. The method includes administering to a patient in need a therapeutically effective amount of the preceding compound of general formula (I), the pharmaceutically acceptable salt, ester, deuterated compound or stereoisomer thereof, the preceding pharmaceutical formulation or the preceding pharmaceutical composition, where the disease refers to a disease mediated by USP1 and a disease related thereto. The disease mediated by USP1 and the disease related thereto are selected from cancer or benign tumor.

The present disclosure provides a preparation method of the above compound, which includes, but is not limited to the following method, and reaction equations of the preparation method are as follows:

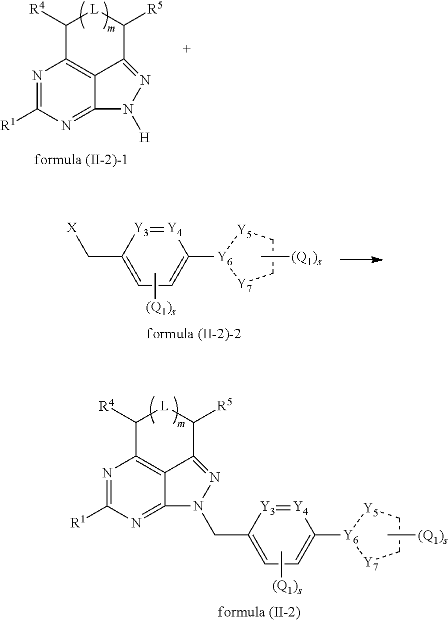

The compound of formula (II-2)-1 is dissolved in an appropriate solvent (such as DMF), added with an alkaline reagent (such as NaH, potassium carbonate, cesium carbonate and sodium carbonate), and then added with the compound of formula (II-2)-2 and reacted for 1-15 hours. The reaction system is added with water for quenching, extracted with an appropriate organic solvent (such as dichloromethane and ethyl acetate), and spin-dried to obtain the compound of general formula (II-2) by an appropriate method (such as silica gel column chromatography).

The preparation method above may be simply summarized as: reacting the compound of formula (II-2)-1 with the compound of formula (II-2)-2 to obtain the compound of general formula (II-2);
wherein, X is halogen; and each Q₁, each Q₂, each L, m, R¹, R⁴, R⁵, Y₃, Y₄, Y₅, Y₆, Y₇, each R^{a}, each R^{b} and each R^{c} are as defined in any of the preceding technical solutions.

The present disclosure also provides an intermediate for preparing compounds of general formula (II-1) to general formula (IX-1), pharmaceutically acceptable salts, esters thereof, deuterated compounds thereof or stereoisomers thereof, which has the following structural formula:

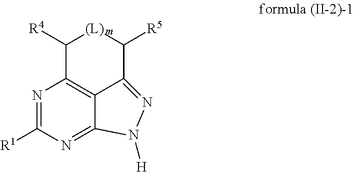

wherein, R¹, R⁴, R⁵, each Q₁, each Q₂, each L, each R^{a}, each R^{b}, each W and m are as defined in any of the preceding technical solutions.

The present disclosure also provides an intermediate for preparing compounds of general formula (III) to general formula (IX-1), pharmaceutically acceptable salts, esters thereof, deuterated compounds thereof or stereoisomers thereof, which has the following structural formula:

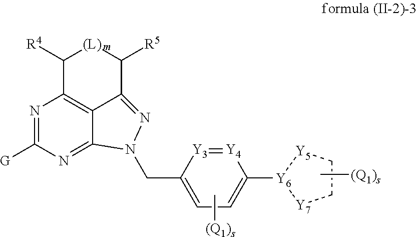

wherein, G is selected from halogen, hydroxyl, amino, C₁₋₆ alkylthio or C₁₋₆ alkylsulfonyl; and
each Q₁, each Q₂, each L, m, R¹, R⁴, R⁵, Y₃, Y₄, Y₅, Y₆, Y₇, each R^{a}, each R^{b} and each R^{c} are as defined in any of the preceding technical solutions.

In some embodiments, G is selected from halogen, hydroxyl, amino, methylthio or methylsulfonyl.

In the specification and claims of the present disclosure, the compounds are named according to the chemical structural formulae, and if it is indicated that the name and chemical structural formula of the compound are inconsistent when the same compound is represented, the chemical structural formula is prevailing.

In the present disclosure, the scientific and technical nouns used herein have the meanings commonly understood by those skilled in the art, unless otherwise stated, however, in order to better understand the present disclosure, definitions of some terms are provided below. When the definitions and explanations of terms provided in the present disclosure are inconsistent with those commonly understood by those skilled in the art, the definitions and explanations of terms provided in the present disclosure are prevailing.

The “halogen” in the present disclosure refers to fluorine, chlorine, bromine and iodine, preferably fluorine and chlorine.

“Halogenation” in the present disclosure means that any hydrogen in the substituent can be substituted by one or more identical or different halogens. “Halogen” is as defined above.

The “C₁₋₆ alkyl” in the present disclosure refers to a linear or branched alkyl containing 1-6 carbon atoms, including, for example, “C₁₋₅ alkyl”, “C₁₋₄ alkyl”, “C₁₋₃ alkyl”, “C₁₋₂ alkyl”, “C₂₋₆ alkyl”, “C₂₋₅ alkyl”, “C₂₋₄ alkyl”, “C₂₋₃ alkyl”, “C₃₋₆ alkyl”, “C₃₋₅ alkyl”, “C₃₋₄ alkyl”, “C₄₋₆ alkyl”, “C₄₋₅ alkyl”, “C₅₋₆ alkyl”, and the like. Specific examples include but are not limited to: methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, and the like. The “C₁₋₄ alkyl” according to the present disclosure refers to a specific example of C₁₋₆ alkyl containing 1-4 carbon atoms.

The “C₁₋₆ alkylene” in the present disclosure refers to a group formed by removing one hydrogen atom from the C₁₋₆ alkyl, including, for example, “C₁₋₅ alkylene”, “C₁₋₄ alkylene”, “C₁₋₃ alkylene”, “C₁₋₂ alkylene”, “C₂₋₆ alkylene”, “C₂₋₅ alkylene”, “C₂₋₄ alkylene”, “C₂₋₃ alkylene”, “C₃₋₆ alkylene”, “C₃₋₅ alkylene”, “C₃₋₄ alkylene”, “C₄₋₆ alkylene”, “C₄₋₅ alkylene”, “C₅₋₆ alkylene”, and the like. Specific examples include but are not limited to: methylene, ethylene, propylene, butylene, pentylene, hexylene, and the like. The “C₁₋₄ alkylene” according to the present disclosure refers to a specific example of C₁₋₆ alkylene containing 1-4 carbon atoms.

The “C₂₋₆ alkenyl” according to the present disclosure refers to a linear or branched or cyclic alkenyl with 2-6 carbon atoms and containing at least one double bond, including, for example, “C₂₋₅ alkenyl”, “C₂₋₄ alkenyl” and “C₂₋₃ alkenyl”. Specific examples include, but are not limited to, ethenyl, 1-propenyl, 2-propenyl, 2-butenyl, 3-butenyl, 2-methyl-1-propenyl, 1-methyl-2-propenyl, 1-pentenyl, 2-pentenyl, 3-pentenyl, 2-methyl-1-butenyl, 3-methyl-1-butenyl, 2-methyl-3-butenyl, 1,1-dimethyl-2-propenyl, 1-ethyl-2-propenyl, 2-hexenyl, 3-hexenyl, 2-methyl-1-pentenyl, 3-methyl-1-pentenyl, 1-methyl-2-pentenyl, 3-methyl-2-pentenyl, 2-methyl-3-pentenyl, 1-methyl-4-pentenyl, 3-methyl-4-pentenyl, 1,1-dimethyl-3-butenyl, 1,2-dimethyl-3-butenyl, 1,3-dimethyl-2-butenyl, 2,2-dimethyl-3-butenyl, 2,3-dimethyl-2-butenyl, 2,3-dimethyl-1-butenyl, 2-ethyl-1-butenyl, 2-ethyl-3-butenyl, etc.

The “C₂₋₆ alkynyl” according to the present disclosure refers to a linear or branched or cyclic alkynyl with 2-6 carbon atoms and containing a triple bond, including, for example, “C₂₋₅ alkynyl”, “C₂₋₄ alkynyl” and “C₂₋₃ alkynyl”. Specific examples include, but are not limited to, ethynyl, 1-propynyl, 2-butynyl, 1-methyl-2-propynyl, 2-pentynoyl, 3-pentynoyl, 1-methyl-2-butynyl, 2-methyl-3-butynyl, 1,1-dimethyl-2-propynyl, 1-ethyl-2-propynyl, 2-hexynyl, 3-hexynyl, 1-methyl-2-pentynoyl, 1-methyl-3-pentynoyl, 2-methyl-3-pentynoyl, 1,1-dimethyl-3-butynyl, 2-ethyl-3-butynyl, etc.

As used herein, the “C₁₋₆ alkoxy, C₁₋₆ alkylamino, di(C₁₋₆ alkyl)amino, C₁₋₆ alkylaminoacyl, C₁₋₆ alkylamido, C₁₋₆ alkylsulfonyl, C₁₋₆ alkylsulfonamido, C₁₋₆ alkylaminosulfonyl, C₁₋₆ alkylcarbonyl, C₁₋₆ alkoxycarbonyl and C₁₋₆ alkylthio” refer to groups formed in the form of C₁₋₆ alkyl-O—, C₁₋₆ alkyl-NH—, (C₁₋₆ alkyl)₂—N—, C₁₋₆ alkyl-NH—C(O)—, C₁₋₆ alkyl-C(O)—NH—, C₁₋₆ alkyl-S(O)₂—, C₁₋₆ alkyl-S(O)₂—NH—, C₁₋₆ alkyl-NH—S(O)₂—, C₁₋₆ alkyl-C(O)—, C₁₋₆ alkyl-O—C(O)— and C₁₋₆ alkyl-S—, where the “C₁₋₆ alkyl” is as defined above.

As used herein, the “C₁₋₄ alkoxy, C₁₋₄ alkylamino, di(C₁₋₄ alkyl)amino, C₁₋₄ alkylaminoacyl, C₁₋₄ alkylamido, C₁₋₄ alkylsulfonyl, C₁₋₄ alkylsulfonamido, C₁₋₄ alkylaminosulfonyl, C₁₋₄ alkylcarbonyl, C₁₋₄ alkoxycarbonyl and C₁₋₄ alkylthio” refer to groups formed in the form of C₁₋₄ alkyl-O—, C₁₋₄ alkyl-NH—, (C₁₋₄ alkyl)₂—N—, C₁₋₄ alkyl-NH—C(O)—, C₁₋₄ alkyl-C(O)—NH—, C₁₋₄ alkyl-S(O)₂—, C₁₋₄ alkyl-S(O)₂—NH—, C₁₋₄ alkyl-NH—S(O)₂—, C₁₋₄ alkyl-C(O)—, C₁₋₄ alkyl-O—C(O)— and C₁₋₄ alkyl-S—, wherein the “C₁₋₄ alkyl” is as defined above.

As used herein, the “halogenated C₁₋₆ alkyl, hydroxy C₁₋₆ alkyl, amino C₁₋₆ alkyl, carboxyl C₁₋₆ alkyl, halogenated C₁₋₆ alkylene and halogenated C₁₋₆ alkoxy” refer to groups formed by one or more (for example, 1-4, 1-3 and 1-2) halogen atoms, hydroxyls, aminos and carboxyls substituting hydrogen atoms in C₁₋₆ alkyl, C₁₋₆ alkylene and C₁₋₆ alkoxy respectively.

As used herein, the “halogenated C₁₋₄ alkyl, hydroxy C₁₋₄ alkyl, amino C₁₋₄ alkyl, carboxyl C₁₋₄ alkyl, halogenated C₁₋₄ alkylene and halogenated C₁₋₄ alkoxy” refer to groups formed by one or more (for example, 1-4, 1-3 and 1-2) halogen atoms, hydroxyls, aminos and carboxyls substituting hydrogen atoms in C₁₋₄ alkyl, C₁₋₄ alkylene and C₁₋₄ alkoxy respectively.

The “3-12 membered cycloalkyl” according to the present disclosure refers to a saturated or partially saturated cyclic alkyl containing 3-12 carbon atoms and having no aromaticity, including “monocycloalkyl” and “fused cycloalkyl”.

The “monocycloalkyl” according to the present disclosure refers to a saturated or partially saturated monocyclic alkyl and having no aromaticity, including “3-8 membered saturated cycloalkyl” and “3-8 membered partially saturated cycloalkyl”; preferably “3-4 membered cycloalkyl”, “3-5 membered cycloalkyl”, “3-6 membered cycloalkyl”, “3-7 membered cycloalkyl”, “4-5 membered cycloalkyl”, “4-6 membered cycloalkyl”, “4-7 membered cycloalkyl”, “4-8 membered cycloalkyl”, “5-6 membered cycloalkyl”, “5-7 membered cycloalkyl”, “5-8 membered cycloalkyl”, “6-7 membered cycloalkyl”, “6-8 membered cycloalkyl”, “7-8 membered cycloalkyl”, “3-6 membered saturated cycloalkyl”, “4-7 membered saturated cycloalkyl”, “4-8 membered saturated cycloalkyl”, “5-8 membered saturated cycloalkyl”, “5-7 membered saturated cycloalkyl”, “5-6 membered saturated cycloalkyl”, “3-6 membered partially saturated cycloalkyl”, “4-7 membered partially saturated cycloalkyl”, “4-8 membered partially saturated cycloalkyl”, “5-8 membered partially saturated cycloalkyl”, “5-7 membered partially saturated cycloalkyl”, and “5-6 membered partially saturated cycloalkyl”, and the like. Specific examples of the “3-8 membered saturated cycloalkyl” include, but are not limited to: cyclopropyl, cyclobutyl, cyclopentyl, cyclohexyl, cycloheptyl, cyclooctyl and the like; and specific examples of the “3-8 membered partially saturated cycloalkyl” include, but are not limited to, cyclopropenyl, cyclobutenyl, cyclopentenyl, cyclopentadienyl, cyclohexenyl, cyclohex-1,3-dienyl, cyclohex-1,4-dienyl, cycloheptenyl, cyclohept-1,3-dienyl, cyclohept-1,4-dienyl, cyclohept-1,3,5 -trienyl, cyclooctenyl, cyclooct-1,3-dienyl, cyclooct-1,4-dienyl, cyclooct-1,5-dienyl, cyclooct-1,3,5-trienyl, cyclooctatetraenyl and the like.

The “fused cycloalkyl” according to the present disclosure refers to a saturated or partially saturated and non-aromatic cycloalkyl, which is formed by two or more cyclic structures sharing two adjacent carbon atoms, where one ring of the fused cycloalkyl may be an aromatic ring, but the fused cycloalkyl as a whole is non-aromatic. The fused cycloalkyl may be fused in a manner as 5-6 membered cycloalkano 5-6 membered cycloalkyl, benzo 5-6 membered cycloalkyl, benzo 5-6 membered saturated cycloalkyl and the like. Examples of the fused cycloalkyl include, but are not limited to, bicyclo[3.1.0]hexyl, bicyclo[4.1.0]heptyl, bicyclo [2.2.0]hexyl, bicyclo [3.2.0]heptyl, bicyclo [4.2.0]octyl, octahydro cyclopentadienyl, octahydro-1H-indenyl, decahydronaphthyl, tetradecahydrophenanthryl, bicyclo[3.1.0]hex-2-enyl, bicyclo[4.1.0]hept-3-enyl, bicyclo[3.2.0]hept-3-enyl, bicyclo[4.2.0]oct-3-enyl, 1,2,3,3a-tetrahydrocyclopentadienyl, 2,3,3a,4,7,7a-hexahydro-1H-indenyl, 1,2,3,4,4a,5,6,8a-octahydronaphthyl, 1,2,4a,5,6,8a-hexahydronaphthyl, 1,2,3,4,5,6,7,8,9,10-decahydrophenanthryl, benzocyclopentyl, benzocyclohexyl, benzocyclohexenyl, benzocyclopentenyl and the like.

The “3-12 membered heterocyclyl” according to the present disclosure refers to a saturated or partially saturated and non-aromatic monocyclic or fused cycloalkyl containing at least one heteroatom (for example, containing 1, 2, 3, 4 or 5) and having 3-12 ring atoms. The heteroatom is a nitrogen atom, an oxygen atom and/or a sulfur atom, and optionally, a ring atom in a ring structure (for example, a carbon atom, a nitrogen atom or a sulfur atom) can be substituted by oxygen. The “3-12 membered heterocyclyl” according to the present disclosure includes “3-12 membered saturated heterocyclyl” and “3-12 membered partially saturated heterocyclyl”. Preferably, the “3-12 membered heterocyclyl” according to the present disclosure includes 1-3 heteroatoms; preferably, the “3-12 membered heterocyclyl” according to the present disclosure contains 1-2 heteroatoms, and the heteroatoms are selected from nitrogen atoms and/or oxygen atoms; and preferably, the “3-12 membered heterocyclyl” according to the present disclosure contains 1-2 nitrogen atoms. The “3-12 membered heterocyclyl” is preferably “3-10 membered heterocyclyl”, “3-8 membered heterocyclyl”, “4-8 membered heterocyclyl”, “3-6 membered heterocyclyl”, “3-6 membered saturated heterocyclyl”, “3-6 membered nitrogen-containing heterocyclyl”, “3-6 membered saturated nitrogen-containing heterocyclyl”, “5-6 membered heterocyclyl”, “5-6 membered saturated heterocyclyl”, “5-6 membered nitrogen-containing heterocyclyl”, etc. Specific examples of the “3-12 membered heterocyclyl” include, but are not limited to: aziridinyl, 2H-aziridinyl, diazaziridinyl, 3H-diazacyclopropenyl, azetidinyl, 1,4-dioxanyl, 1,3-dioxanyl, 1,3-dioxolanyl, 1,4-dioxadienyl, tetrahydrofuranyl, tetrahydropyranyl, dihydropyrrolidyl, pyrrolidinyl, imidazolyl, 4,5-dihydroimidazolyl, pyrazolidinyl, 4,5-dihydropyrazolyl, 2,5-dihydrothienyl, tetrahydrothiophenyl, 4,5-dihydrothiazolyl, piperidinyl, piperazinyl, morpholinyl, 4,5-dihydrooxazolyl, 4,5-dihydroisoxazolyl, 2,3-dihydroisoxazolyl, 2H-1,2-oxazinyl, 6H-1,3-oxazinyl, 4H-1,3-thiazinyl, 6H-1,3-thiazinyl, 2H-pyranyl, 2H-pyran-2-onyl, 3,4-dihydro-2H-pyranyl, pyrrolidinocyclopropyl, cyclopentazacyclopropyl, pyrrolidinocyclobutyl, pyrrolidinopyrrolidinyl, pyrrolidinopiperidinyl, pyrrolidinopiperazinyl, pyrrolidinomorpholinyl, piperidinomorpholinyl, benzopyrrolidinyl, benzocyclopentyl, benzocyclohexyl, benzotetrahydrofuranyl, benzopyrrolidinyl, benzoimidazolyl, benzoxazolidinyl, benzothiazolidinyl, benzoisoxazolidinyl, benzoisothiazolidinyl, benzopiperidinyl, benzomorpholinyl, benzopiperazinyl, benzotetrahydropyranyl, pyridinocyclopentyl, pyridinocyclohexyl, pyridinotetrahydrofuranyl pyridinopyrrolidinyl, pyridinoimidazolyl, pyridoxazolidinyl, pyridinothiazolidinyl, pyridinosatoisothiazolidinyl, pyridino is othiazo lidinyl, pyridinopiperidinyl, pyridinomorpholinyl, pyridinopiperazinyl, pyridinotetrahydropyranyl, pyrimidinocyclopentyl, pyrimidinocyclohexyl, pyrimidinotetrahydrofuranyl, pyrimidinopyrrolidinyl, pyrimidinoimidazolyl, pyrimidinothiazolidinyl, pyrimidinoisothiazolidinyl, pyrimidinoisothiazolidinyl, pyrimidinopiperidiny, pyrimidinomorpholinyl, pyrimidinopiperazinyl, pyrimidinotetrahydropyranyl, tetrahydroimidazo[4,5-c]pyridinyl, 3,4-dihydroquinazo linyl, 1,2-dihydroquinoxalinyl, benzo[d][1,3]dioxolyl, 2H-chromenyl, 2H-chromen-2-onyl, 4H-chromenyl, 4H-chromen-4-onyl, 4H-1,3-benzoxazinyl, 4,6-dihydro-1H-furo[3,4-d]imidazolyl, 3a,4,6,6a-tetrahydro-1H-furo[3,4-d]imidazolyl, 4,6-dihydro-1H-thieno[3,4-d]imidazolyl, 4,6-dihydro-1H-pyrrolo[3,4-d]imidazolyl, octahydrobenzo octahydro-benzo[d]imidazolyl, decahydroquinolinyl, hexahydrothienoimidazolyl, hexahydrofuroimidazolyl, 4,5,6,7-tetrahydro-1H-benzo[d]imidazolyl, octahydrocyclopenta[c]pyrrolyl, 4H-1,3-benzoxazinyl and the like.

The “6-10 membered aryl” in the present disclosure refers to an aromatic ring group which contains 6 to 10 cyclic carbon atoms, including “6-8 membered monocycloaryl ” and “8-10 membered fused ring aryl”.

The “6-8 membered monocycloaryl” in the present disclosure refers to monocycloaryl which contains 6 to 8 cyclic carbon atoms, and examples thereof comprise, but are not limited to, phenyl, cyclooctatetraenyl and the like; and phenyl is preferred.

The “8-10 membered fused ring aryl” in the present disclosure refers to an unsaturated aromatic ring group which is formed by two or more ring structures sharing two adjacent atoms with each other and contains 8 to 10 cyclic carbon atoms, and “9-10 membered fused ring aryl” is preferred, with specific examples such as naphthyl and the like.

The “5-12 membered heteroaryl” in the present disclosure refers to an aromatic ring group which contains 5 to 12 ring atoms (where at least one ring atom is a heteroatom, such as a nitrogen atom, an oxygen atom or a sulfur atom), such as 5-12 membered nitrogen-containing heteroaryl, 5-12 membered oxygen-containing heteroaryl, 5-12 membered sulfur-containing heteroaryl and the like, including “5-8 membered monoheteroaryl” and “8-10 membered fused heteroaryl”.

The “5-8 membered monoheteroaryl” in the present disclosure refers to an aromatic monocyclic ring group which contains 5 to 8 ring atoms (where at least one ring atom is a heteroatom, such as a nitrogen atom, an oxygen atom or a sulfur atom). Optionally, ring atoms (such as carbon atoms, nitrogen atoms or sulfur atoms) in a ring structure may be substituted by oxygen atoms. The “5-8 membered monoheteroaryl” includes, for example, “5-7 membered monoheteroaryl”, “5-6 membered monoheteroaryl”, “5-6 membered nitrogen-containing monoheteroaryl”, “5 membered nitrogen-containing monoheteroaryl” and the like. Specific examples of “5-8 membered monocyclic heteroaryl” comprise, but are not limited to, furyl, thienyl, pyrrolyl, thiazolyl, isothiazolyl, thiadiazole, oxazolyl, isoxazole, oxadiazolyl, imidazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, pyridyl, 2-pyridonyl, 4-pyridonyl, pyrimidinyl, pyridazinyl, pyrazinyl, 1,2,3-triazinyl, 1,3,5-triazinyl, 1,2,4,5-tetrazinyl, aza-cycloheptatrienyl, 1,3-diaza-cycloheptatrienyl, aza-cyclooctatetraenyl. The “5-6 membered heteroaryl” refers to a specific example in which 5-8 member heteroaryl contains 5-6 ring atoms.

The “8-10 membered fused heteroaryl” in the present disclosure refers to an unsaturated aromatic ring structure which is formed by two or more ring structures sharing two adjacent atoms with each other and contains 8 to 10 ring atoms (where at least one ring atom is a heteroatom, such as a nitrogen atom, an oxygen atom or a sulfur atom). Optionally, ring atoms (such as carbon atoms, nitrogen atoms or sulfur atoms) in a ring structure may be substituted by oxygen atoms, including “9-10 membered fused heteroaryl”, “8-9 membered fused heteroaryl” and the like, and a fusion mode may be benzo 5-6 membered heteroaryl, 5-6 membered heteroaro 5-6 membered heterocyclyl and the like; and specific examples comprise, but are not limited to, pyrrolopyrrole, pyrrolofuran, pyrazolopyrrole, pyrazolothiophene, furanothiophene, pyrazoloxazole, benzofuryl, benzoisofuryl, benzothiophenyl, indolyl, isoindolyl, benzoxazolyl, benzimidazolyl, indazolyl, benzotriazolyl, quinolyl, 2-quinolinonyl, 4-quinolinonyl, 1-isoquinolinonyl, isoquinolinyl, acridinyl, phenanthridinyl, benzopyridazinyl, phthalazinyl, quinazolinyl, quinoxalinyl, purinyl, naphthyridinyl and the like.

The “optionally substituted by . . . ” in the present disclosure includes two conditions of being “substituted” and “unsubstituted”.

The-- and - in the present disclosure is selected from a single-bond or a double-bond.

Unless otherwise specified, any atom of the compounds of the present disclosure may represent any stable isotope of the atom. Unless otherwise specified, when a certain site in a structure is defined as H, which is namely deuterium (H-1), this site only contains naturally occurring isotopes. Similarly, unless otherwise specified, when a certain site in a structure is defined as D, which is namely deuterium (H-2), an isotope content at this site is at least 3,340 folds greater than the naturally occurring isotope content (0.015%) (which means that at least 50.1% deuterium isotopes are contained), and when one or more sites in structures of the compounds of the present disclosure are defined as D, which is namely deuterium (H-2), contents of the compounds represented by the structures may be at least 52.5%, at least 60%, at least 67.5%, at least 75%, at least 82.5%, at least 90%, at least 95%, at 97%, at least 98.5%, at least 99% and at least 99.5%.

Deuteration rates of the compounds of the present disclosure refer to ratios of labeled-synthesized isotope contents to naturally occurring isotope contents. A deuteration rate of each designated deuterium atom in the compounds of the present disclosure may be at least 3,500 folds (52.5%), at least 4,000 folds (60%), at least 4,500 folds (67.5%), at least 5,000 folds (75%), at least 5,500 folds (82.5%), at least 6,000 folds (90%), at least 6,333.3 folds (95%), at least 6,466.7 folds (97%), at least 6,566.7 folds (98.5%), at least 6,600 folds (99%) and at least 6,633.3 folds (99.5%).

Isotopologues in the present disclosure refer to compounds that differ only in isotopic composition in chemical structure. The compounds containing deuterium at specific sites in the present disclosure may also contain very few hydrogen isotopologues at the sites, and an amount of hydrogen isotopologues at deuterated sites in the deuterated compounds of the present disclosure depends on many factors, including a deuterium isotope purity of a deuterated reagent (D₂O, D₂, NaBD₄, L1AID₄, etc.) and an effectiveness of a synthetic method of introducing deuterium isotopes. However, as mentioned above, a total amount of hydrogen isotopologues at deuterated sites will be less than 49.9%. The total amount of hydrogen isotopologues at deuterated sites in the deuterated compounds of the present disclosure will be less than 47.5%, 40%, 32.5%, 25%, 17.5%, 10%, 5%, 3%, 1% or 0.5%.

In the present disclosure, any atom not designated as deuterium exists in the natural isotope abundance.

The “deuterated” in the present disclosure refers to replacing one or more hydrogen atoms on a deuterated group by one or more deuterium atoms, which may be partially deuterated or completely deuterated. For example, a deuterated compound may contain only one deuterium atom. In some embodiments, the deuterated compound contains only two deuterium atoms. In some embodiments, the deuterated compound contains only three deuterium atoms. In some embodiments, the deuterated compound contains only four deuterium atoms.

The “optionally deuterated” as mentioned in the present disclosure includes two conditions that a group is deuterated and non-deuterated, where the “deuterated” is as defined above.

The “therapeutically effective amount” as mentioned in the present disclosure refers to amounts of the aforementioned compound, the pharmaceutical formulation and the pharmaceutical composition capable of at least alleviating symptoms of a disease of a patient when administered to the patient. An actual amount including the “therapeutically effective amount” may vary according to various conditions, including but being not limited to a specific disease treated, a severity of disease, a physical and health status of patient and a route of administration. A skilled medical practitioner may easily determine an appropriate amount by methods known in the medical field.

Beneficial Effects of the Present Disclosure

(1) The compound, the pharmaceutically acceptable salt, ester and deuterated compound thereof, or the stereoisomer thereof of the present disclosure have excellent inhibitory activity on USP1, and can treat and/or prevent the disease mediated by USP1 and the disease related thereto;

(2) the compound of the present disclosure has a good inhibitory effect on tumor cells;

(3) the compound, the pharmaceutically acceptable salt, ester and deuterated compound thereof, or the stereoisomer thereof of the present disclosure have good pharmacokinetic properties, more lasting action and high bioavailability;

(4) the compound, the pharmaceutically acceptable salt, esters and deuterated compound thereof, or the stereoisomer thereof of the present disclosure have good safety; and

(5) the compound of the present disclosure has simple preparation process, high drug purity and stable quality, and are easy for large-scale industrial production.

The beneficial effects of the compound provided by the examples of the present disclosure are further illustrated by experiments, which should not be understood that the compounds provided by the examples of the present disclosure only have the following beneficial effects.

Experimental Example 1 In-vitro Enzymatic Activities of Compounds of the Present Disclosure

Test compounds: the compounds synthesized in the examples of the present disclosure, with structural formulas shown in Table 1.

Experimental Reagents:

| Reagent | Vendor | Cat No. |
|---|---|---|
| Recombinant Human His6-USP1/His6-UAF1 | R&D | E-568-050 |
| Complex Protein | | |
| Ubiquitin Rhodamine 110 Protein, CF (Ub-Rho) | R&D | U-555-050 |

Experimentalcomsumables:

| Consumables | Vendor | Cat No. |
|---|---|---|
| 384-Well plate | Perkin Elmer | 6007279 |

First Experimental Method:

1. Compound Dilution

1) The compounds of the present disclosure were prepared to 10 mM with DMSO, and used as test stock solutions.

2) The stock solutions of the compounds of the present disclosure were diluted in a 4-fold gradient for 10 concentrations, with a maximum concentration of 10 mM.

3) The diluted compounds of the present disclosure were respectively transferred to 384-well plates (diluted by 1,000 folds) by Echo550, 2 multiple wells were set for each concentration, and a final concentration of DMSO was 1%.

4) The final concentrations of test compounds were 10,000 nM, 2,500 nM, 625 nM, 156 nM, 39 nM, 9.8 nM, 2.4 nM, 0.61 nM, 0.15 nM and 0.038 nM.

2. Enzyme Reaction Experiment

1) An enzyme solution was prepared in 1×test buffer.

2) Ubiquitin Rhodamine 110 protein, CF (UB-Rho), was added into 1×determination buffer to prepare a substrate solution.

3) 10 μL of enzyme solution and 1×reaction buffer were transferred to a 384-well plate, and

4) incubated at room temperature for 15 minutes.

5) 10 μL of substrate solution was added into each well to start the reaction, centrifuged for 30 seconds, and shaken for 30 seconds.

3. Result Detection

1) Plate reading was carried out on SpectraMax Paradigm for 30 minutes, with an excitation wavelength of 480 nm and an emission wavelength of 540 nm.

2) Data on SpectraMax Paradigm were collected.

4. Data analysis

An inhibition (% inh) was calculated by the following formula:

$$inhibition ( \% ) = 100 \% \times \frac{Max - Signal}{Max - Min}$$

wherein, Max represented: a luminous signal intensity of a positive control well without adding the compound;
Min represented: a luminous signal intensity of a negative control well without adding the enzyme; and
Signal represented: a luminous signal intensity of the compound of the test sample.

IC₅₀ was Calculated by the Following Formula:

$$Y = Bottom + \frac{Top - Bottom}{1 + ( IC 50 \div X ) \times HillSlope}$$

wherein, Y represented: % inhibition; and
X represented: the concentration of the compound.

Experimental Results:

TABLE 2
Inhibitory activities of compounds
of the present disclosure on USP-1

| Compounds | IC₅₀ (nM) |
|---|---|
| Compound 1 | 28.0 |
| Compound 2 | 5.0 |
| Compound 3 | 18 |
| Compound 11 | 22 |

It can be seen from the above experimental results that the compounds prepared by the present disclosure can effectively inhibit the activity of USP1, thus being effective USP1 inhibitors.

Second Experimental Method:

In the enzyme reaction experiment, the incubation time at room temperature in the step 4) was changed to 60 minutes, other conditions were the same as those in the first experimental method, and the following test results were obtained:

TABLE 3
Inhibitory activities of compounds
of the present disclosure on USP-1

| Compounds | IC₅₀ (nM) |
|---|---|
| Compound 16 | 8.00 |
| Compound 18 | 3.98 |
| Compound 19 | 4.36 |
| Compound 1-1 | 12.88 |
| Compound 11-1 | 15.0 |
| Compound 16-1 | 6.67 |
| Compound 17-1 | 6.66 |

It can be seen from the above experimental results that the compounds prepared by the present disclosure can effectively inhibit the activity of USP1, thus being effective USP1 inhibitors.

Experimental Example 2 First Experiment of in-vitro Cytological Inhibitory Activities of Compounds of the Present Disclosure

Test compounds: some compounds of the present disclosure, with chemical names and structures shown in the preparation examples.

A cell line used in the following experiment was as follows: MDA-MB-436: human breast cancer cells.

The definitions represented by the following abbreviations were as follows:

FBS: fetal bovine serum

ITS-G: insulin-transferrin-selenium additive

Glutathione: GSH

Experimental method (CelltiterGlo assay)

1 Cell Preparation

1.1 Cell Culture:

All cells were adherent cells, the medium was DMEM+10% FBS+1% ITS-G+16 μg/ml glutathione, and the cells were tested in a logarithmic growth phase.

1.2 Preparation of Cell Suspension:

Cells in the logarithmic growth phase were collected and counted by a platelet counter. The cell viability was detected by a trypan blue exclusion method, and the cell viability was kept over 90%. The cell concentration was adjusted to be in a proper range, and 90 μL of cell suspension was added into a 96-well plate respectively.

TABLE 4
Number of cell inoculation

| Cell line | Number of cells inoculated |
|---|---|
| MDA-MB-436 | 4,000 cells/well |

2 Preparation of Test Compounds

2.1 DMSO stock solutions of the test compounds were prepared, and concentrations of the stock solutions of the test compounds were 10 mM respectively.

2.2 Preparation of Working Stock Solutions of Test Compounds

The stock solutions of the test compounds were serially diluted from 10 mM with DMSO in a 3-fold gradient for a total of 8 concentrations. Then, 2 □L of DMSO-gradient diluted compounds were respectively added into 198 □L of culture solutions to give the working stock solutions of the test compounds (a compound concentration was 10-fold higher than the final concentration, and a maximum concentration was 10 μM).

2.3 Compound Treatment

10 μL of compound working stock solution was added into each well in the 96-well plate inoculated with cells (10-fold dilution, a final DMSO concentration was 0.1%).

The final concentrations of the test compounds were: 10,000.00 nM, 3,333.33 nM, 1,111.11 nM, 370.37 nM, 123.46 nM, 41.15 nM, 13.72 nM and 4.57 nM.

2.4 Setting of Control Well

Solvent control: 0.1% DMSO.

Blank control: only a medium was added, and cells were not inoculated.

2.5 The 96-well plate was placed in a 5% CO₂ cell incubator at 37° C. to culture for 7 days.

3 Detection

A CTG reagent was thawed and the 96-well plate was equilibrated to room temperature for 30 minutes, 60 μL of reagent (Celititer Glo assay kit) was added into each well, shaking was carried out for 2 minutes with a shaker (in the absence of light), and the plate was incubated at room temperature for 20 minutes (in the absence of light). Light signal values were read on a multi-functional microplate reader.

4 Data Processing

1) inhibition (%)=(DMSO solvent control well reading−test compound well reading)/(DMSO solvent control well reading−blank control well reading)×100%;

2) GraphPad Prism was input for drawing to obtain a curve and IC₅₀.

Experimental Result and Conclusion

TABLE 5
In-vitro cytological activities of compounds
of the present disclosure (IC₅₀, nM)

| Test compound | MDA-MB-436 |
|---|---|
| Compound 1 | 68.05 |
| Compound 2 | 88.12 |
| Compound 11 | 100.95 |

It can be seen from Table 5 that the compounds of the present disclosure can effectively inhibit the proliferation of MDA-MB-436 cells, indicating that the compounds of the present disclosure have the potential of clinical application in treating cancerous diseases with BRCA1 gene mutation.

Experimental Example 3 Second Experiment of in-vitro Cytological Inhibitory Activities of Compounds of the Present Disclosure

Test compounds: some compounds of the present disclosure, with chemical names and structures shown in the preparation examples.

Cell lines used in the following experiment were as follows: MDA-MB-436: human breast cancer cells; and Caov-3: Human ovarian cancer cells

First Experimental Method (CelltiterGlo Assay)

1 Cell Preparation

1.1 Cell Culture:

All cells were adherent cells, the MDA-MB-436 cell medium was DMEM+10% FBS+1% ITS-G+16 μg/ml glutathione, the Caov-3 cell medium was DMEM+10% FBS, and cells were tested in a logarithmic growth phase.

1.2 Preparation of Cell Suspension:

Cells in the logarithmic growth phase were collected and counted by a platelet counter. The cell viability was detected by a trypan blue exclusion method, and the cell viability was kept over 90%. The cell concentration was adjusted to be in a proper range, and 90 μL of cell suspension was added into a 96-well plate respectively.

TABLE 6
Number of cell inoculation

| Cell line | Number of cells inoculated |
|---|---|
| MDA-MB-436 | 3,000 cells/well |
| Caov-3 | 2,000 cells/well |

2 Preparation of Test Compounds

2.1 DMSO stock solutions of the test compounds were prepared, and concentrations of the stock solutions of the test compounds were 10 mM respectively.

2.2 Preparation of Working Stock Solutions of Test Compounds

The stock solutions of the test compounds were serially diluted from 10 mM with DMSO in a 3-fold gradient for a total of 8 concentrations. Then, 2 □L of DMSO-gradient diluted compounds were respectively added into 198 □L of culture solutions to give the working stock solutions of the test compounds (a compound concentration was 10-fold higher than the final concentration, and a maximum concentration was 100 μM).

2.3 Compound Treatment

10 μL of compound working stock solution was added into each well in the 96-well plate inoculated with cells (10-fold dilution, a final DMSO concentration was 0.1%).

The final concentrations of the test compounds were: 10,000.00 nM, 3,333.33 nM, 1,111.11 nM, 370.37 nM, 123.46 nM, 41.15 nM, 13.72 nM and 4.57 nM.

2.4 Setting of Control Well

Solvent control: 0.1% DMSO.

Blank control: 0 hour after dosing, the 96-well plate was detected and read.

2.5 The 96-well plate was placed in a 5% CO₂ cell incubator at 37° C. to culture for 7 days.

3 Detection

A CTG reagent was thawed and the 96-well plate was equilibrated to room temperature for 30 minutes, 60 μL of reagent (Celititer Glo assay kit) was added into each well, shaking was carried out for 2 minutes with a shaker (in the absence of light), and the plate was incubated at room temperature for 20 minutes (in the absence of light). Light signal values were read on a multi-functional microplate reader.

4. Data Processing

1) inhibition (%)=(DMSO solvent control well reading−test compound well reading)/(DMSO solvent control well reading−blank control well reading)×100%;

2) GraphPad Prism was input for drawing to obtain a curve and IC₅₀.

Experimental Result and Conclusion

TABLE 7
In-vitro cytological activities of compounds
of the present disclosure (IC₅₀, nM)

| Test compound | MDA-MB-436 |
|---|---|
| Compound 16 | 42.31 |
| Compound 18 | 29.67 |
| Compound 19 | 49.79 |
| Compound 1-1 | 42.43 |
| Compound 11-1 | 121.69 |
| Compound 16-1 | 55.32 |
| Compound 17-1 | 110.41 |

The IC₅₀ values of the inhibitory activities of the compounds 11, 16, 18, 19, 1-1, 11-1, 16-1 and 17-1 in the examples of the present disclosure on Caov-3 cells range from 1 nM to 600 nM, indicating that the compounds of the present disclosure can effectively inhibit the proliferation of the MDA-MB-436 cells and the Caov-3 cells, so that the compounds of the present disclosure have the clinical application potential for treating HRD positive (homologous recombination defect) cancer diseases.

Second Experimental Method:

The concentrations of the compound stock solutions prepared were 5 mM.

The stock solutions of the test compounds were serially diluted from 5 mM with DMSO in a 3-fold gradient for a total of 9 concentrations.

The final concentrations of the test compounds were: 10,000.00 nM, 3,333.33 nM, 1,111.11 nM, 370.37 nM, 123.46 nM, 41.15 nM, 13.72 nM, 4.57 nM and 1.52 nM.

Other conditions were the same as those in the first experimental method, and the IC₅₀ value of the inhibitory activity of the compound 20 on the MDA-MB-436 cells was 55.7 nM; and the IC₅₀ value of the inhibitory activity of the compound 20 on the Caov-3 cells was 1 nM to 100 nM.

Experiment Example 4 Pharmacokinetic Experiment of Compounds of the Present Disclosure

1. Test compounds: the compounds in the examples of the present disclosure, prepared according to the examples in the specification of the present disclosure.

| Number of | | | |
|---|---|---|---|
| animals | Sex | Administration route | Sample |
| 9 | Female | Intravenous injection (iv) | Plasma |
| 9 | Female | Oral administration (po) | Plasma |

2. Preparation of Test Compound Solution

(1) Intravenous administration (iv): 2.54 mg of compound 11 was taken, added with 0.495 ml of DMSO solution to be dissolved by vortex and ultrasound, added with 0.495 ml of PEG400 to be mixed by vortex, and then added with 1.484 ml of 28% HP-β-CD to be mixed by vortex to obtain a clear solution, with a concentration of 1 mg/ml.

(2) Oral administration (po): 3.33 mg of compound 11 was taken, added with 3.243 ml of solvent (2% HPC+0.1% Tween 80), and ground evenly to obtain a uniform suspension, with a concentration of 1 mg/ml.

3. Experimental Method

(1) Administration

The test compounds were administered intravenously (iv), with an administration dosage of 5 mg/kg and an administration volume of 5 ml/kg.

The test compounds were administered orally (po), with an administration dosage of 10 mg/kg and an administration volume of 10 ml/kg.

(2) Blood Sampling

0.083 hour, 0.25 hour, 0.5 hour, 1 hour, 2 hours, 3 hours, 4 hours, 6 hours, 8 hours and 24 hours after administration, tail vein blood sampling was carried out, about 100 μl of whole blood was taken at each time point, plasma was centrifugally separated in a high-speed centrifuge at 8,000 rpm for 6 minutes, and the plasma was frozen in a refrigerator at −80° C.

(3) Analysis of Plasma Sample

A protein precipitation method was adopted: 20 μl of plasma was added into a 96-well deep-well plate, added with 200 μl of internal standard solution (tolbutamide-200 ng/ml) to be processed by vortex for 10 minutes, and then centrifuged at 4,000 rpm for 20 minutes, and 100 μl of supernatant was taken, and added with 100 μL of water to be processed by vortex for 3 minutes. LC-MS/MS was to be analyzed.

4. Experimental Result and Conclusion

Experimental data show that intravenous injection or oral administration of the compounds of the present disclosure has high exposure in vivo and suitable half-life and clearance rate, showing good pharmacokinetic properties. For example, the exposure of the compound 11 administered intravenously in mice is more than 6,000 h*ng/ml, indicating that the compounds in the examples of the present disclosure have good clinical application prospects.

### DETAILED DESCRIPTION OF THE EMBODIMENTS

The following clearly describes the technical solutions of the present disclosure with reference to the embodiments of the present disclosure. Apparently, the described embodiments are merely some but not all of the embodiments of the present disclosure. Based on the embodiments of the present disclosure, all other embodiments obtained by those of ordinary skills in the art without going through any creative work shall fall within the protection scope of the present disclosure.

The abbreviations used in the following experiments have the following meanings:

Xphos-Pd-G2: Chloro(2-dicyclohexylphosphino-2′,4′,6′-triisopropyl-1,1′-biphenyl)[2-(2′-amino-1,1′-biphenyl)]palladium (II); Xphos: 2-dicyclohexylphosphino-2′,4′,6′-triisopropylbiphenyl; Pd(dppf)Cl₂: dichloro[1,1′-bis(diphenylphosphino)ferrocene]palladium; DMF: N,N-dimethylformamide EA: ethyl acetate; PE: petroleum ether; DCM: dichloromethane; NMP: N-methylpyrrolidone; and DIBAl-H: diisobutylaluminum hydride

Example 1 Preparation of 4-(4-cyclopropyl-6-methoxypyrimidine-5-yl)-2-(4-(1-methyl-4-(trifluoromethyl)-1H-imidazol-2-yl)benzyl)-2,6,7,8- tetrahydropyrazolo[3,4,5-de]quinazoline (Compound 1)

(1) Preparation of 2-(bis(methylthio)methylene)cyclohexane-1,3-dione

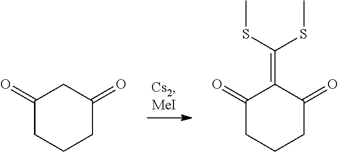

1-3-cyclohexanedione (20 g, 178.4 mmol) was dissolved to DMF (200 mL), added with potassium carbonate (74 g, 535.2 mmol), and reacted at 20° C. for 0.5 h. Carbon disulphid (20 g, 267.6 mmol) was added and reacted at 20° C. for 1 h. Methyl iodide (76 g, 535.2 mmol) was added and reacted at 20° C. for 1 h. The reaction mixture was concentrated to give a crude product used directly in next step.

(2) Preparation of 2-amino-4-(methylthio)-7,8-dihydroquinazo line-5 (6H)-one

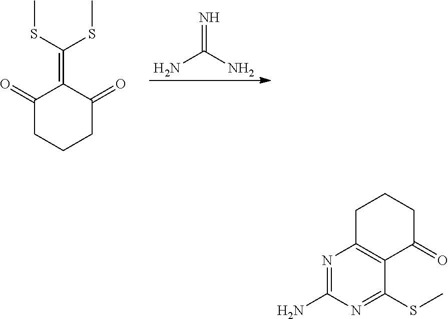

2-(bis(methylthio)methylene)cyclohexane-1,3-dione (crude product from the above step) was dissolved in DMF (200 mL), added with guanidine hydrochloride (17 g, 178.4 mmol) and potassium carbonate (49.3 g, 356.8 mmol), reacted at 100° C. for 16 h, cooled to 25° C., added with water (300 mL), and filtered to obtain a filter cake, and then dried in vacuum to give 16.3 g of the target compound, with a yield of 44%.

(3) Preparation of 2-chloro-4-(methylthio)-7,8-dihydroquinazolin-5(6H)-one

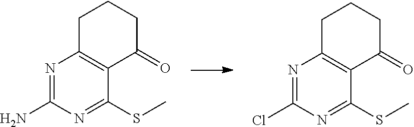

2-amino-4-(methylthio)-7,8-dihydroquinazoline-5(6H)-one (16.3 g, 78 mmol) was dissolved to dichloromethane (150 mL), added with titanium tetrachloride (14.8 g, 78 mmol) and tert-butyl nitrite (48 g, 468 mmol), and reacted at 25° C. for 3 h. Water was added for quenching, and the reaction mixture was filtered to obtain a filtrate, concentrated and subjected to column chromatography (ethyl acetate/petroleum ether=0-40%) to give 6.1 g of the target compound, with a yield of 34%.

(4) Preparation of 2-(4-cyclopropyl-6-methoxypyrimidin-5-yl)-4-(methylthio)-

7,8-dihydroquinazolin-5(6H)-one

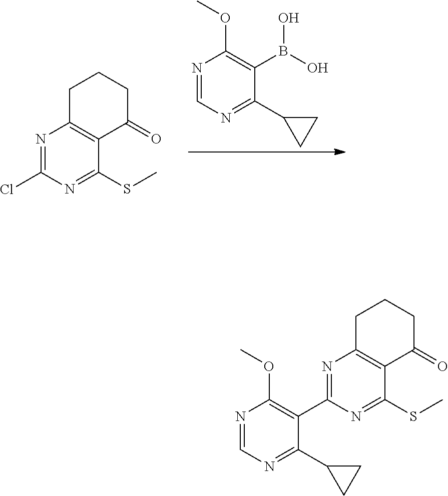 [SMILES: CSc1nc(Cl)nc2c1C(=O)CCC2 ; COc1ncnc(C2CC2)c1B(O)O ; COc1ncnc(C2CC2)c1-c1nc2c(c(SC)n1)C(=O)CCC2]

2-chloro-4-(methylthio)-7,8-dihydroquinazolin-5(6H)-one (1.2 g, 5.15 mmol) was dissolved to 1,4-dioxane (30 mL), added with (4-cyclopropyl-6-methoxypyrimidin-5-yl)boric acid (1 g, 5.15 mmol), Xphos-Pd-G2 (408 mg, 0.52 mmol), Xphos (496 mg, 1.04 mmol), K₃PO₄ (1.2 g, 5.7 mmol), and water (10 mL), and reacted under the protection of N₂ at 90° C. for 3 h. After the reaction, the solvent was spin-dried to give 830 mg of the target compound by normal phase preparation and separation (ethyl acetate/petroleum ether=0-50%) with a yield of 47%.

(5) Preparation of 2-(4-cyclopropyl-6-methoxypyrimidin-5-yl)-4-hydrazino-7,8-dihydroquinazolin-5(6H)-one

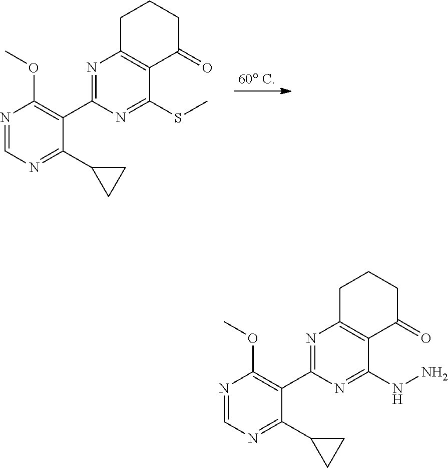 [SMILES: COc1ncnc(C2CC2)c1-c1nc2c(c(SC)n1)C(=O)CCC2 ; COc1ncnc(C2CC2)c1-c1nc2c(c(NN)n1)C(=O)CCC2]

2-(4-cyclopropyl-6-methoxypyrimidin-5-yl)-4-(methylthio)-7,8-dihydroquinazolin-5(6H)-one (830 mg, 2.4 mmol) was dissolved to ethanol (20 mL), added with hydrazine hydrate (184 mg, 3.6 mmol), and reacted at 70° C. for 3 h. After the reaction, the reaction mixture was concentrated to give 700 mg of the target compound with a yield of 89%.

(6) Preparation of 4-(4-cyclopropyl-6-methoxypyrimidin-5-yl)-2,6,7,8-tetrahydropyrazolo[3,4,5-de]quinazoline

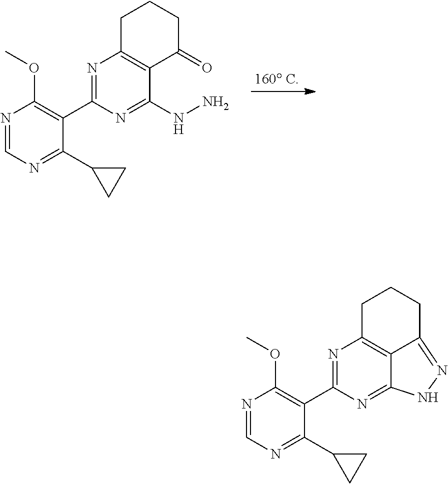 [SMILES: COc1ncnc(C2CC2)c1-c1nc2c(c(NN)n1)C(=O)CCC2 ; COc1ncnc(C2CC2)c1-c1nc2c3c(n[nH]c3n1)CCC2]

2-(4-cyclopropyl-6-methoxypyrimidin-5-yl)-4-hydrazino-7,8-dihydroquinazolin-5(6H)-one (300 mg, 0.92 mmol) was dissolved to N-methylpyrrolidone (5 mL) and ethanol (60 mL), and reacted at 160° C. for 1 h with microwave. The reaction mixture was subjected to column chromatography separation (methanol/water=0-60%) to give 150 mg of the target compound with a yielding of 53%.

(7) Preparation of 4-(4-cyclopropyl-6-methoxypyrimidine-5-yl)-2-(4-(1-methyl-4-(trifluoromethyl)-1H-imidazol-2-yl)benzyl)-2,6,7,8-tetrahydropyrazolo[3,4,5-de]quinazoline

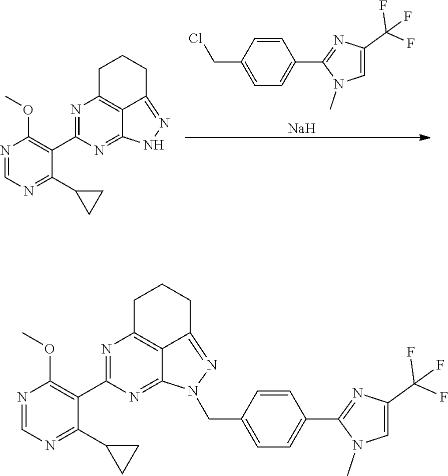

4-(4-cyclopropyl-6-methoxypyrimidin-5-yl)-2,6,7,8-tetrahydropyrazolo[3,4,5-de]quinazoline (130 mg, 0.43 mmol) was dissolved to N,N-dimethylformamide (4 mL), added with sodium hydride (60%, 34 mg, 0.85 mmol), and reacted at 20° C. for 10 min, then added with 2-(4-(chloromethyl)phenyl)-1-methyl-4-(trifluoromethyl)-1H-imidazole (139 mg, 0.51 mmol), and reacted at 20° C. for 3 h. After the reaction, the reaction mixture was subjected to column chromatography separation (methanol/water=0-40%) to give 18.5 mg of the target compound with a yield of 8%.

Molecular formula: C₂₈H₂₅F₃N₈O, Molecular weight: 546.6, LC-MS (M/e): 547.2(M+H⁺)

¹H-NMR (400 MHz, CDCl₃) δ: 8.65(s, 1H), 7.60-7.45 (m, 4H), 7.29(s, 1H), 5.67(s, 2H), 3.91(s,3H), 3.72(s,3H), 3.15-3.02 (m, 4H), 2.45-2.32 (m, 2H), 1.69-1.55 (m, 1H), 1.25-1.19 (m, 2H), 0.92-0.75 (m, 2H).

Example 2 Preparation of 4-(3-fluoro-2-isopropylphenyl)-2-(4-(1-methyl-4-(trifluoromethyl)-1H-imidazol-2-yl)benzyl)-2,6,7,8-tetrahydropyrazolo[3,4,5- de]quinazoline (Compound 2)

(1) Preparation of 3-fluoro-2-(prop-1-en-2-yl)phenol

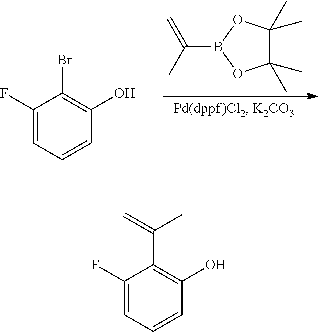

2-bromo-3-fluorophenol (11 g, 57.6 mmol), 4,4,5,5-tetramethyl-2-(prop-1-en-2-yl)-1,3,2-dioxolane (13.2 g, 78.6 mmol), K₂CO₃ (15.9 g, 115.2 mmol) and Pd(dppf)Cl₂ (4.2 g, 5.7 mmol) were dissolved in dioxane (120 mL) and water (24 mL), reacted at 80° C. for 16 h under the protection of nitrogen. After the completion of the reaction by LCMS detection, the reaction solution was poured into water (200 mL), extracted with EA, then organic phases were dried with anhydrous sodium sulfate, spin-dried and separated by silica gel column chromatography (EA:PE=1:5) to give 5.6 g of the product with a yield of 63.9%.

(2) Preparation of 3-fluoro-2-isopropylphenol

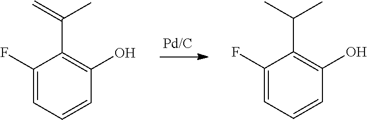

3-fluoro-2-(prop-1-en-2-yl)phenol (5.6 g, 36.8 mmol) was dissolved to methanol (60 mL), added with Pd/C (1.2 g), and reacted at 25° C. for 4 h. After the completion of the reaction by LCMS detection, the reaction mixture was subjected to suction filtration, and the filtrate was spin-dried to give 5 g of the product with a yield of 88.2%.

(3) Preparation of 3-fluoro-2-isopropylphenyl triflate

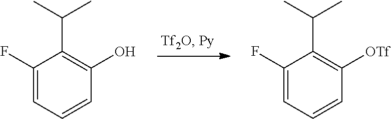 [SMILES: CC(C)c1c(O)cccc1F ; CC(C)c1c(F)cccc1OS(=O)(=O)C(F)(F)F]

3-fluoro-2-isopropylphenol (1 g, 6.49 mmol) was dissolved to dichloromethane (20 mL), dropwise added with pyridine (1.0 g, 12.6 mmol) and Tf₂O (2.7 g, 9.6 mmol) at −10° C., and then continuously reacted for 1 h. After the completion of the reaction by LCMS detection, the reaction solution was poured into water (50 mL) for quenching, and extracted with DCM. Organic phases were dried with anhydrous sodium sulfate, spin-dried, and separated by silica gel column chromatography (EA:PE=1:5) to give 1.6 g of the product with a yield of 86.1%.

(4) Preparation of 2-(3-fluoro-2-isopropylphenyl)-4,4,5,5-tetramethyl-1,3,2-dioxolane

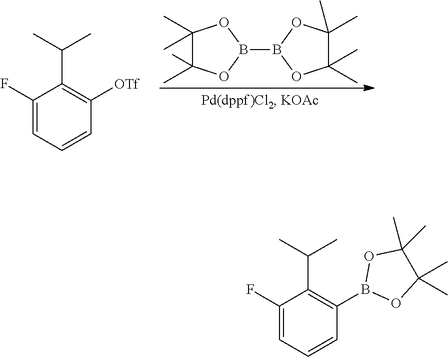

3-fluoro-2-isopropylphenyl triflate (1.6 g, 5.59 mmol), bis(pinacolato)diboron (2.8 g, 11.18 mmol), Pd(dppf)Cl₂ (409 mg, 0.56 mmol) and potassium acetate (1.7 g, 17.32 mmol) were dissolved to dioxane (40 mL), reacted under the protection of nitrogen at 80° C. for 8 h. After the completion of the reaction by LCMS detection, the reaction solution was spin-dried, and extracted with EA. Organic phases were spin-dried and separated by silica gel column chromatography (EA:PE=1:5) to give 1 g of the product with a yield of 67.8%.

(5) Preparation of 2-(3-fluoro-2-isopropylphenyl)-4-(methylthio)-7,8-dihydroquinazolin-5(6H)-one

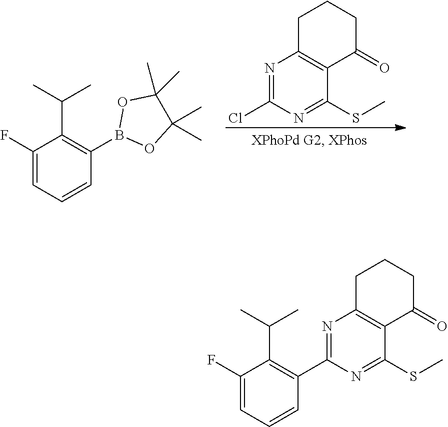

2-(3-fluoro-2-isopropylphenyl)-4,4,5,5-tetramethyl-1,3,2-dioxolane (1.0 g, 3.79 mmol), 2-chloro-4-(methylthio)-7,8-dihydroquinazolin-5(6H)-one (870 mg, 3.79 mmol), XPhosPdG2 (299 mg, 0.38 mmol), XPhos (362 mg, 0.76 mmol) and potassium phosphate (892 mg, 4.2 mmol) were dissolved to dioxane (26 mL) and water (7 mL), and reacted under the protection of nitrogen at 90° C. for 2 h. After the completion of the reaction by LCMS detection, the reaction solution was spin-dried and separated by silica gel column chromatography (EA:PE=1:2) to give 350 mg of the product with a yield of 28.0%.

(6) Preparation of 2-(3-fluoro-2-isopropylphenyl)-4-hydrazino-7,8-dihydroquinazolin-5(6H)-one

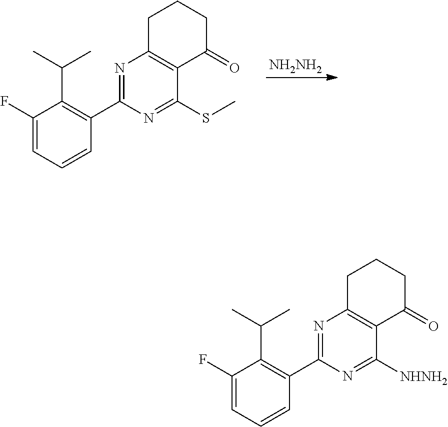

2-(3-fluoro-2-isopropylphenyl)-4-(methylthio)-7,8-dihydroquinazolin-5(6H)-one (350 mg, 1.06 mmol) was dissolved to ethanol (10 mL), added with hydrazine hydrate (64 mg, 1.27 mmol), and reacted at 70° C. for 8 h. After the completion of the reaction by LCMS detection, the reaction solution was spin-dried and directly used for next reaction.

(7) Preparation of 4-(3-fluoro-2-isopropylphenyl)-2,6,7,8-tetrahydropyrazolo[3,4,5-de]quinazoline

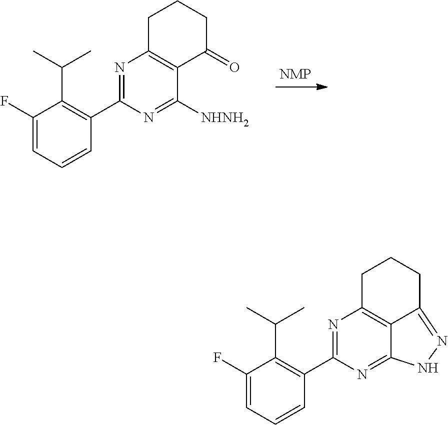 [SMILES: CC(C)c1c(F)cccc1-c1nc2c(c(NN)n1)C(=O)CCC2 ; CC(C)c1c(F)cccc1-c1nc2c3c(n[nH]c3n1)CCC2]

2-(3-fluoro-2-isopropylphenyl)-4-hydrazino-7,8-dihydroquinazolin-5(6H)-one (crude product) was dissolved to NMP (2 mL), and reacted at 160° C. for 1 h with microwaves. After the completion of the reaction by LCMS detection, the reaction mixture was subjected to reversed-phase chromatography purification (MeOH:H₂O=0-50) to give 100 mg of the product with a two-step yield of 31.8%.

(8) Preparation of 4-(3-fluoro-2-isopropylphenyl)-2-(4-(1-methyl-4-(trifluoromethyl)-1H-imidazol-2-yl)benzyl)-2,6,7,8-tetrahydropyrazolo[3,4,5-de]quinazoline

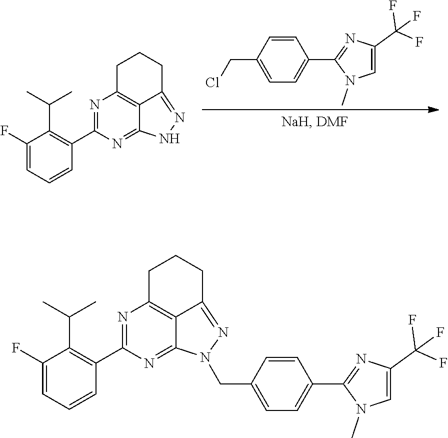 [SMILES: CC(C)c1c(F)cccc1-c1nc2c3c(n[nH]c3n1)CCC2 ; Cn1cc(C(F)(F)F)nc1-c1ccc(CCl)cc1 ; CC(C)c1c(F)cccc1-c1nc2c3c(nn(Cc4ccc(-c5nc(C(F)(F)F)cn5C)cc4)c3n1)CCC2]

4-(3-fluoro-2-isopropylphenyl)-2,6,7,8-tetrahydropyrazolo[3,4,5-de]quinazoline (70 mg, 0.24 mmol) was dissolved to DMF (3 mL), added with NaH (60%, 19 mg, 0.48 mmol) in batches at 25° C., added with 2-(4-(chloromethyl)phenyl)-1-methyl-4-(trifluoromethyl)-1H-imidazole (77 mg, 0.28 mmol) after 10 min, and continuously reacted for 4 h. The reaction was completed by LCMS detection. The reaction solution was added with water (20 mL) for quenching, and extracted with dichloromethane. Organic phases were spin-dried, and the crude product was separated by silica gel column chromatography (EA:PE=1:1) to give 16 mg of the product with a yield of 12.5%.

Molecular formula: C₂₉H₂₆F₄N₆ Molecular weight: 534.6 LC-MS (M/e): 535.0 (M+H⁺)

¹H-NMR(400 MHz,DMSO) δ: 7.90 (s, 1H), 7.60-7.70 (m, 2H), 7.40-7.50 (m,2H), 7.18-7.35 (m, 3H), 5.63 (s, 2H), 3.73 (s, 3H), 3.15-3.22 (m, 1H), 2.95-3.05 (m,4H), 2.20-2.30 (m, 2H), 1.25 (d, J=6.8 Hz, 6H).

Example 3 Preparation of 4-(3-fluoro-2-isopropylphenyl)-2-(4-(5-methyl-3-(trifluoromethyl)-1H-pyrazol-1-yl)benzyl)-2,6,7,8-tetrahydropyrazolo[3,4,5-de]quinazoline (Compound 3)

(1) Preparation of 4-(5-methyl-3-(trifluoromethyl)-1H-pyrazol-1-yl)methyl benzoate

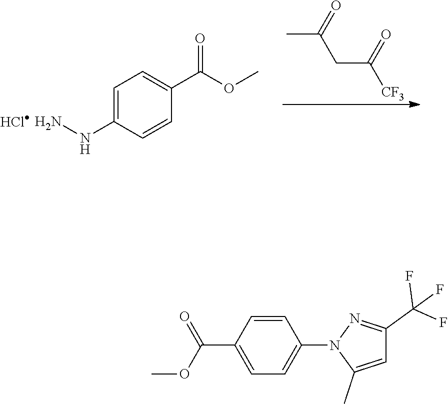

4-hydrazino-methylbenzoate hydrochloride (9.0 g, 44.4 mmol) and 1,1,1-trifluoropropane-2,4-dione (6.8 g, 44.1 mmol) were dissolved to hexafluoroisopropanol (70 mL), dropwise added with triethylamine (8.9 g, 87.9 mmol) at 0° C., and then reacted at 20° C. for 2 h. The system was spin-dried, and the residue was purified by silica gel column chromatography (PE:EA=10:1) to give the product (9.0 g, with a yield of 71.3%).

(2) Preparation of (4-(5-methyl-3-(trifluoromethyl)-1H-pyrazol-1-y1)phenyl)methanol

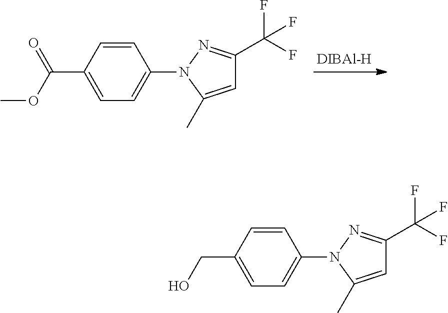

4-(5-methyl-3-(trifluoromethyl)-1H-pyrazol-1-yl)methyl benzoate (5.0 g, 17.6 mmol) was dissolved in THF (60 mL), dropwise added with DIBA1-H (1.5 mol/L, 46.9 mL, 70.4 mmol), and reacted at 20° C. for 2 h after the addition was completed. Water was slowly added for quenching, added with saturated aqueous ammonium chloride solution (50 mL) and EA (150 mL) to extract for solution separation. Organic phases were spun-dried, and the residue was purified by silica gel column chromatography (PE:EA=3:1) to give the product (4.0 g, with a yielding of 88.7%).

(3) Preparation of 1-(4-(chloromethyl)phenyl)-5-methyl-3-(trilluoromethyl)-1H-pyrazole

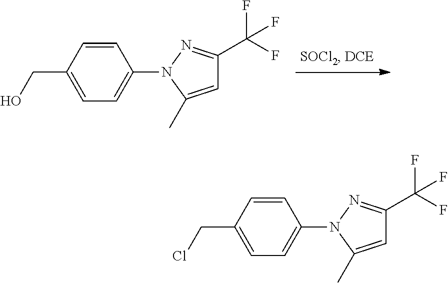 [SMILES: Cc1cc(C(F)(F)F)nn1-c1ccc(CO)cc1 ; Cc1cc(C(F)(F)F)nn1-c1ccc(CCl)cc1]

(4-(5-methyl-3-(trifluoromethyl)-1H-pyrazol-1-yl)phenyl)methanol (3.7 g, 14.4 mmol) was dissolved to dichloroethane (30 mL), added with sulfur dichloride (3 mL), and then reacted at 50° C. for 1 h. The system was spin-dried, and the residue was purified by silica gel column chromatography (PE:EA=5:1) to give the product (3.6 g, with a yielding of 91.0%).

(4) Preparation of 4-(3-fluoro-2-isopropylphenyl)-2-(4-(5-methyl-3-(trifluoromethyl)-1H-pyrazol-1-yl)benzyl)-2,6,7,8-tetrahydropyrazolo[3,4,5-de]quinazoline

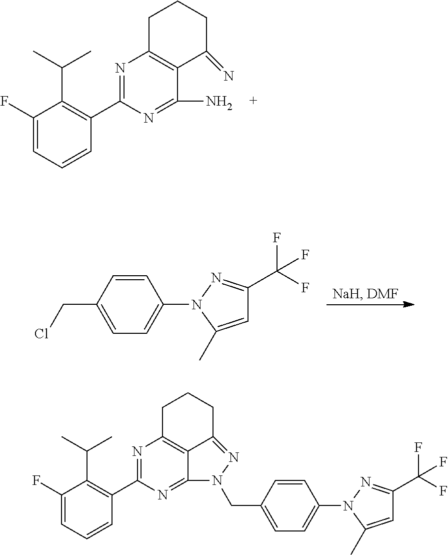

4-(3-fluoro-2-isopropylphenyl)-2,6,7,8-tetrahydropyrazolo[3,4,5-de]quinazoline (100 mg, 0.34 mmol) was dissolved to DMF (5 mL), and added with NaH (60%, 27 mg, 0.68 mmol) in batches at 25° C., then added with 1-(4-(chloromethyl)phenyl)-5-methyl-3-(trifluoromethyl)-1H-pyrazole (139 mg, 0.51 mmol) after 30 min, and continuously reacted for 2 h. Water (20 mL) was added to the reaction solution for quenching, then added with EA (30 mL) for extraction and solution separation. Organic phases were spin-dried, and the residue was purified by silica gel column chromatography (PE:EA=3:1) to give 20 mg of crude product, which was subjected to C₁₋₈ reversed purification (water/methanol=2/8) and freeze-dried to give the product (6 mg, with a yield of 3.3%).

Molecular formula: C₂₉H₂₆F₄N₆ Molecular weight: 534.6 LC-MS (M/e): 535.0 (M+H⁺)

¹H-NMR(400 MHz, CDCl₃) δ: 7.65-7.61(m,2H), 7.42-7.38(m,2H), 7.36-7.33 (m,1H), 7.27-7.23(m,1H), 7.13-7.08(m,1H), 6.43(s,1H), 5.63(m,2H), 3.18-3.11(m,1H), 3.12-3.03 (m, 4H), 2.44-2.38(m,2H), 2.32(s,3H), 1.36 (d, J=6.8 Hz, 6H).

Example 4 Preparation of 2-(4-(1-methyl-4-(trifluoromethyl)-1H-imidazol-2-yl)benzyl)-4-(4-(trifluoromethyl)pyridin-3-yl)-2,6,7,8-tetrahydropyrazolo[3,4,5-de]quinazoline (Compound 10)

(1) Preparation of (4-(trifluoromethyl)pyridin-3-yl)boric acid

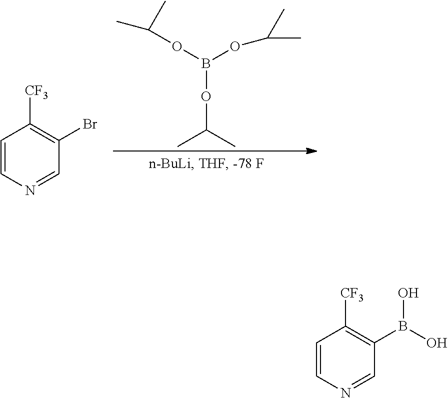

3-bromo-4-(trifluoromethyl)pyridine (9.2 g, 0.041 mol) was dissolved to THF (200 mL), added with boron isopropoxide (10.8 g, 0.057 mol), added with n-BuLi (2.5 M) (22.9 mL, 0.057 mol) at −78° C. and reacted for 3 h. After 50.0 mL of water was added for quenching, the reaction mixture was adjusted to a pH of 4 by 4N hydrochloric acid, then adjusted to a pH of 8 by saturated sodium hydrogen carbonate solution and extracted with ethyl acetate. Organic phases were concentrated to give the crude product directly used in next reaction.

(2) Preparation of 4-(methylthio)-2-(4-(trifluoromethyl)pyridin-3-yl)-7,8-dihydroquinazolin-5(6H)-one

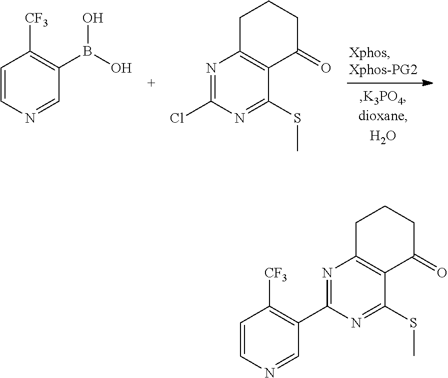 [SMILES: OB(O)c1cnccc1C(F)(F)F ; CSc1nc(Cl)nc2c1C(=O)CCC2 ; CSc1nc(-c2cnccc2C(F)(F)F)nc2c1C(=O)CCC2]

2-chloro-4-(methylthio)-7,8-dihydroquinazolin-5(6H)-one (1.5 g, 6.6 mmol) was dissolved to 1,4-dioxane (75 mL), added with (4-(trifluoromethyl)pyridin-3-yl)boric acid (crude product from the above step), Xphos-Pd-G2 (514.7 mg, 0.66 mmol), Xphos (625.3 mg, 1.3 mmol), K₃PO₄ (1.5 g, 7.2 mmol), and water (25 mL), and reacted under the protection of N₂ at 90° C. for 3 h. After the reaction, the solvent was spin-dried, and the residue was subjected to normal phase preparation and separation (ethyl acetate/petroleum ether=0-50%) to give 535.0 mg of the target product.

(3) Preparation of 4-hydrazino-2-(4-(trifluoromethyl)pyridin-3-yl)-7,8-dihydroquinazolin-5(6H)-one

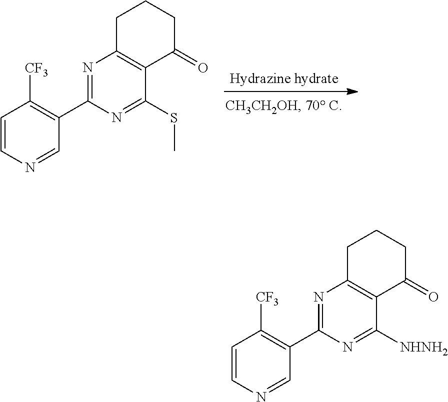 [SMILES: CSc1nc(-c2cnccc2C(F)(F)F)nc2c1C(=O)CCC2 ; NNc1nc(-c2cnccc2C(F)(F)F)nc2c1C(=O)CCC2]

4-(methylthio)-2-(4-(trifluoromethyl)pyridin-3-yl)-7,8-dihydroquinazolin-5(6H)-one (0.48 g, 1.4 mmol) was dissolved to ethanol (20.0 mL), added with hydrazine hydrate (248.0 mg, 5.0 mmol) and reacted at 80° C. for 3 h. After the reaction, the solvent was spin-dried and the residue was subjected to normal phase preparation and separation (ethyl acetate/petroleum ether=0-70%) to give 240.0 mg of the target product with a yielding of 52.2%.

(4) Preparation of 4-(4-(trifluoromethyl)pyridin-3-yl)-2,6,7,8-tetrahydropyrazolo[3,4,5-de]quinazoline

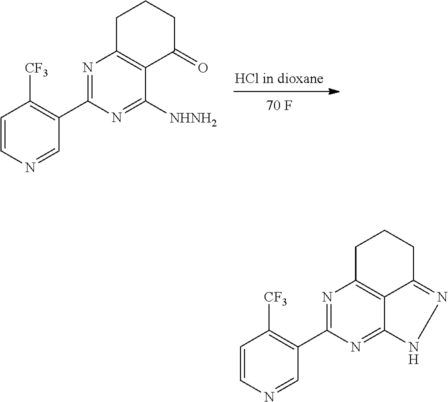 [SMILES: NNc1nc(-c2cnccc2C(F)(F)F)nc2c1C(=O)CCC2 ; FC(F)(F)c1ccncc1-c1nc2c3c(n[nH]c3n1)CCC2]

4-hydrazino-2-(4-(trifluoromethyl)pyridin-3-yl)-7,8-dihydroquinazolin-5(6H)-one (200.0 mg, 0.62 mmol) was dissolved to HCL 1,4-dioxane solution (4 M,18 mL), and reacted at 70° C. for 1 h. After the reaction, the solvent was spin-dried, and the reaction mixture was added with a saturated sodium bicarbonate solution to adjust a pH to neutrality, and then extracted with ethyl acetate. Organic phases were subjected to normal phase preparation and separation (ethyl acetate/petroleum ether=0-50%) to give the product (130.0 mg, with a yield of 68.8%).

(5) Preparation of 2-(4-(1-methyl-4-(trifluoromethyl)-1H-imidazol-2-yl)benzyl)-4-(4-(trifluoromethyl)pyridin-3-yl)-2,6,7,8-tetrahydropyrazolo[3,4,5-de]quinazoline

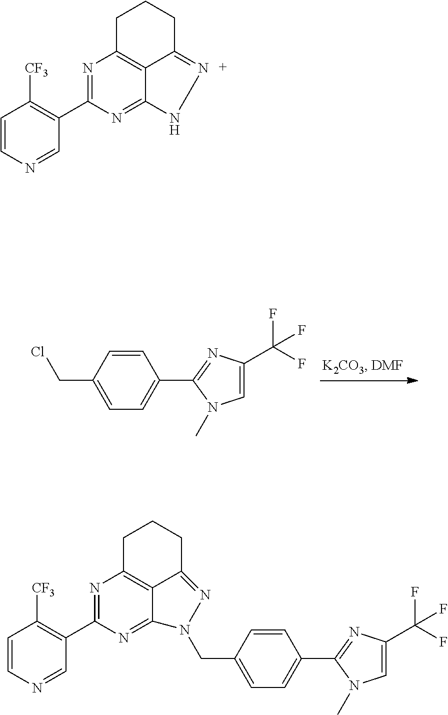 [SMILES: FC(F)(F)c1ccncc1-c1nc2c3c(n[nH]c3n1)CCC2 ; Cn1cc(C(F)(F)F)nc1-c1ccc(CCl)cc1 ; Cn1cc(C(F)(F)F)nc1-c1ccc(Cn2nc3c4c(nc(-c5cnccc5C(F)(F)F)nc42)CCC3)cc1]

4-(4-(trifluoromethyl)pyridin-3-yl)-2,6,7,8-tetrahydropyrazolo[3,4,5-de]quinazoline (105 mg, 0.34 mmol) was dissolved to DMF (4 mL), added with potassium carbonate (143 mg, 1.03 mmol), then added with 2-(4-(chloromethyl)phenyl)-1-methyl-4-(trifluoromethyl)-1H-imidazole (71 mg, 0.26 mmol), and reacted at 50° C. for 5 h. After the reaction, the reaction solution was subjected to normal phase column chromatography separation (methanol/water=0-10%) to give 8.8 mg of the product with a yield of 4.7%.

Molecular formula: C₂₋₆H₁₉F₆N₇ Molecular weight: 543.48 LC-MS (M/e): 544.2(M+H⁺)

¹H-NMR (400 MHz, CDCl₃) δ: 9.13(s, 1H), 7.97(d, 1H), 7.69 (d, 1H), 7.6(d, 2H), 7.5(d, 2H), 7.28(s,1H), 5.63(d,2H), 3.73 (s, 3H), 3.12-3.08 (m, 4H), 2.43-2.37 (m, 2H).

Example 5 Preparation of 4-(4-cyclopropyl-6-methoxypyrimidine-5-yl)-2-(4-(1-methyl-4-(trifluoromethyl)-1H-imidazol-2-yl)benzyl)-6,7,8,9-tetrahydro-2H-1,2,3,5-tetraazabenzo[cd]pyrene (Compound 11)

(1) Preparation of 2-(bis(methylthio)methylene)cycloheptane-1,3-dione

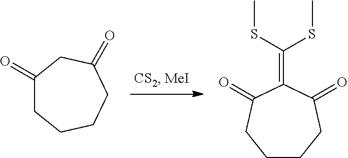

1,3-cycloheptanedione (9 g, 71.4 mmol) was dissolved to DMF (200 mL), added with potassium carbonate (30 g, 214.2 mmol), and reacted at 20° C. for 0.5 h. Carbon disulphid (8.1 g, 106.4 mmol) was added and reacted at 20° C. for 1 h. Methyl iodide (30.4 g, 214.2 mmol) was added and reacted at 20° C. for 1 h. The reaction mixture was concentrated to give a crude product used directly in next step.

(2) Preparation of 2-amino-4-(methylthio)-6,7,8,9-tetrahydro-5H-cyclohepta[d]pyrimidin-5-one

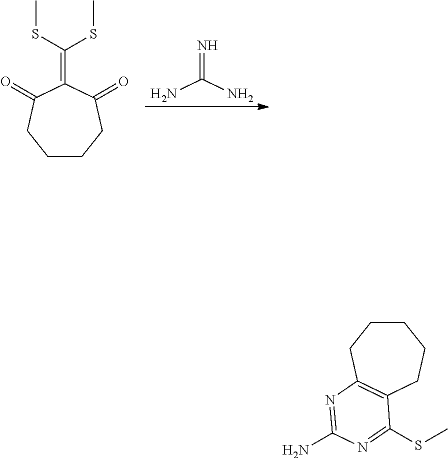

2-(bis(methylthio)methylene)cycloheptane-1,3-dione (crude product from the above step) was dissolved in DMF (200 mL), and added with guanidine hydrochloride (6.8 g, 71.4 mmol) and potassium carbonate (14.8 g, 107.1 mmol), reacted at 100° C. for 16 h, cooled to 25° C., added with water (300 mL), and filtered to obtain a filter cake, and then dried in vacuum to give 10.4 g of the product with a two-step yielding of 65.0%.

(3) Preparation of 2-chloro-4-(methylthio)-6,7,8,9-tetrahydro-5H-cyclohepta[d]pyrimidin-5-one

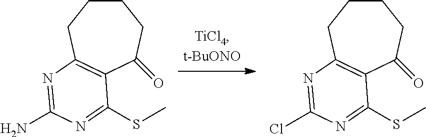

2-amino-4-(methylthio)-6,7,8,9-tetrahydro-5H-cyclohepta[d]pyrimidin-5-one (10.4 g, 46.6 mmol) was dissolved to dichloromethane (150 mL), added with titanium tetrachloride (8.8 g, 46.4 mmol) and tert-butyl nitrite ((28.7 g, 278.6 mmol), and reacted at 25° C. for 3 h. Water was added for quenching, and the reaction mixture was filtered to obtain a filtrate, concentrated and subjected to column chromatography (ethyl acetate/petroleum ether=0-30%) to give 1.2 g of the product with a yield of 10.6%.

(4) Preparation of 2-(4-cyclopropyl-6-methoxypyrimidin-5-yl)-4-(methylthio)-6,7,8,9-tetrahydro-5H-cyclohepta[d]pyrimidin-5-one

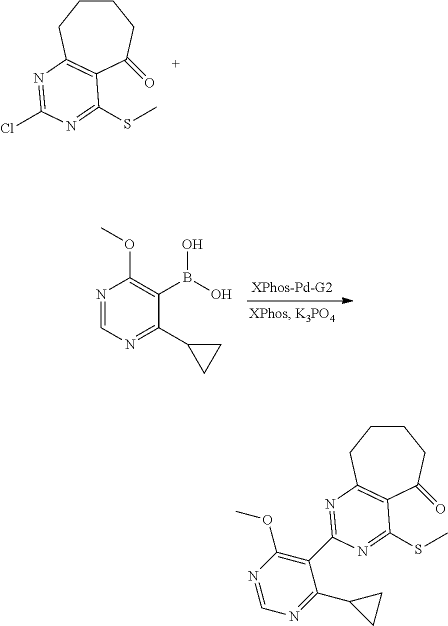

2-chloro-4-(methylthio)-6,7,8,9-tetrahydro-5H-cyclohepta[d]pyrimidin-5-one (800 mg, 3.3 mmol) was dissolved to 1,4-dioxane (18 mL), added with (4-cyclopropyl-6-methoxypyrimidin-5-yl)boric acid (640 mg, 3.3 mmol), Xphos-Pd-G2 (259 mg, 0.33 mmol), Xphos (315 mg, 0.66 mmol), potassium phosphate (770 mg, 3.6 mmol) and water (6 mL), and reacted under the protection of nitrogen at 90° C. for 3 h. After the reaction, the solvent was spin-dried, and the residue was subjected to normal phase preparation and separation (ethyl acetate/petroleum ether=0-50%) to give 400 mg of the product with a yield of 34.0%.

(5) Preparation of 4-(4-cyclopropyl-6-methoxypyrimidine-5-yl)-6,7,8,9-tetrahydro-2H-1,2,3,5-tetraazabenzo[cd]pyrene

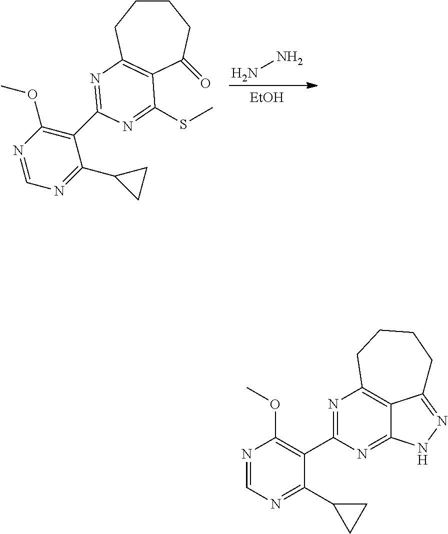

2-(4-cyclopropyl-6-methoxypyrimidin-5-yl)-4-(methylthio)-6,7,8,9-tetrahydro-5H-cyclohepta[d]pyrimidin-5-one (200 mg, 0.56 mmol) was dissolved to ethanol (5 mL), added with hydrazine hydrate (84 mg, 1.69 mmol), and reacted at 80° C. for 3 h. After the reaction, the solvent was spin-dried and the residue was subjected to normal phase preparation and separation (ethyl acetate/petroleum ether=0-70%) to give 150 mg of the product with a yield of 82.9%.

(6) Preparation of 4-(4-cyclopropyl-6-methoxypyrimidine-5-yl)-2-(4-(1-methyl-4-(trifluoromethyl)-1H-imidazol-2-yl)benzyl)-6,7,8,9-tetrahydro-2H-1,2,3,5-tetraazabenzo[cd]pyrene

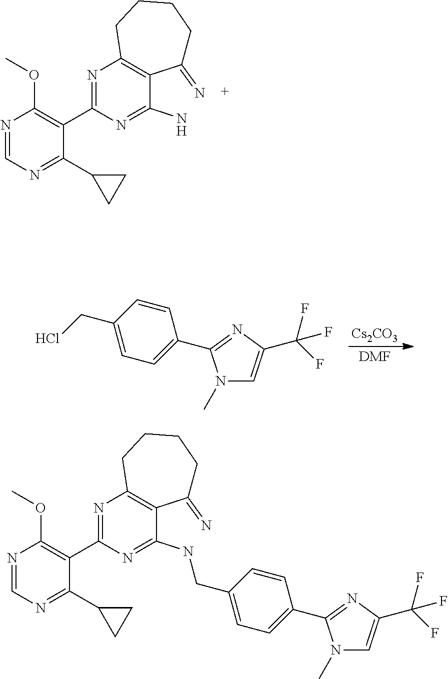

4-(4-cyclopropyl-6-methoxypyrimidine-5-yl)-6,7,8,9-tetrahydro-2H-1,2,3,5-tetraazabenzo[cd]pyrene (70 mg, 0.22 mmol) was dissolved to DMF (4 mL), added with cesium carbonate (143 mg, 0.44 mmol), and reacted at 20° C. for 10 min, then added with 2-(4-(chloromethyl)phenyl)-1-methyl-4-(trifluoromethyl)-1H-imidazole (71 mg, 0.26 mmol), and reacted at 20° C. for 3 h. After the reaction, the reaction solution was subjected to reversed column chromatography separation (methanol/water=0-70%) to give 25 mg of the product with a yield of 20.2%.

Molecular formula: C₂₉H₂₇F₃N₈O Molecular weight: 560.6 LC-MS (M/e): 561.2(M+H⁺)

¹H-NMR (400 MHz, CDCl₃) δ: 8.70(s, 1H), 7.97(s, 1H), 7.92-7.89 (m, 2H), 7.43-7.41(m, 2H), 5.67(s, 2H), 3.92(s,3H), 3.75(s,3H), 3.25-3.08 (m, 4H), 2.07-2.00 (m, 4H), 1.69-1.55 (m, 1H), 1.22-1.18 (m, 2H), 0.95-0.85 (m, 2H).

Example 6 Preparation of 4-(4-cyclopropyl-6-methoxypyrimidine-5-yl)-2-(4-(1-isopropyl-4-(trifluoromethyl)-1H-imidazol-2-yl)benzyl)-2,6,7,8- tetrahydropyrazolo[3,4,5-de]quinazoline (Compound 16)

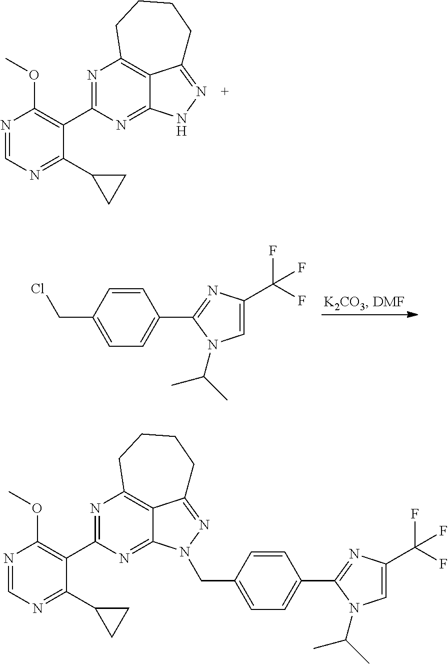

4-(4-cyclopropyl-6-methoxypyrimidin-5-yl)-2,6,7,8-tetrahydropyrazolo[3,4,5 -de]quinazoline (100 mg, 0.32 mmol) was dissolved to DMF (4 mL), added with potassium carbonate (88 mg, 0.64 mmol) and 2-(4-(chloromethyl)phenyl)-1-isopropyl-4-(trifluoromethyl)-1H-imidazole (98 mg, 0.32 mmol), and reacted at 70° C. for 5 h. After the reaction, the reaction mixture was subjected to column chromatography separation (methanol/water=0-40%) to give 21 mg of the product with a yield of 11.4%.

Molecular formula: C₃₀H₂₉F₃N₈O Molecular weight: 574.6 LC-MS (M/e): 575.2(M+H⁺)

¹H-NMR (400 MHz, DMSO): 8.67(s, 1H), 7.53-7.51 (m, 4H), 7.48 (s, 1H), 5.68(s, 2H), 4.56-4.55(m, 1H), 3.95(s,3H), 3.15-3.00 (m, 4H), 2.45-2.40(m,2H), 1.69-1.55 (m, 1H), 1.53-1.32 (m, 6H), 1.42-1.21 (m, 2H) , 0.96-0.87 (m, 2H).

Example 7 Preparation of 4-(1-isopropyl-4-methyl-1H-pyrazol-5-yl)-2-(4-(1-methyl-4-(trifluoromethyl)-1H-imidazol-2-yl)benzyl)-2,6,7,8- tetrahydropyrazolo[3,4,5-de]quinazoline (Compound 18)

(1) Preparation of 2-(1-isopropyl-4-methyl-1H-pyrazol-5-yl)-4-(methylthio)-7,8-dihydroquinazolin-5(6H)-one

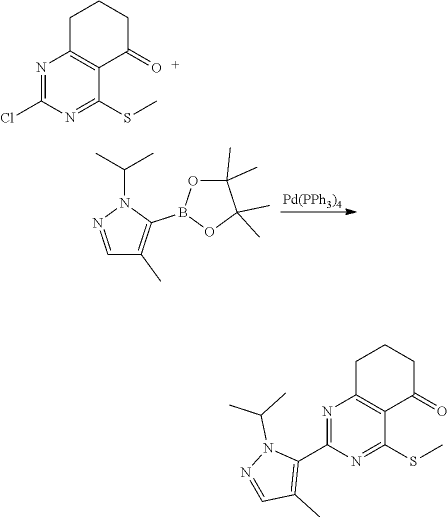

2-chloro-4-(methylthio)-7,8-dihydroquinazolin-5(6H)-one (1.0 g, 4.4 mmol) and 1-isopropyl-4-methyl-5-(4,4,5,5-tetramethyl-1,3,2-dioxaborolan-2-yl)-1H-pyrazole (1.3 g, 5.2 mmol) were dissolved to 1,4-dioxane (20 mL) and water (4 mL), then added with tetratriphenylphosphine palladium (760 mg, 0.66 mmol) and sodium carbonate (950 mg, 9.0 mmol). Under the protection of N₂, the reaction mixture reacted at 90° C. for 3 h. After the reaction, the reaction mixture was concentrated and purified by silica gel column chromatography (ethyl acetate: normal heptane=35%) to give 700 mg of the product with a yield of 50.6%.

(2) Preparation of 4-hydrazino-2-(1-isopropyl-4-methyl-1H-pyrazol-5-yl)-7,8-dihydroquinazolin-5(6H)-one

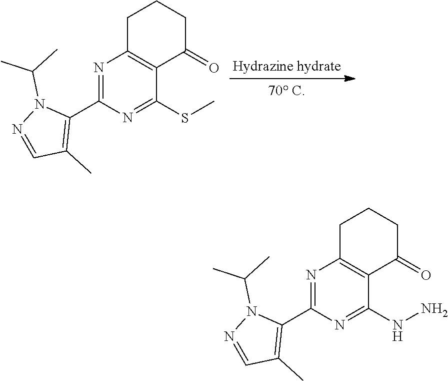 [SMILES: CSc1nc(-c2c(C)cnn2C(C)C)nc2c1C(=O)CCC2 ; Cc1cnn(C(C)C)c1-c1nc2c(c(NN)n1)C(=O)CCC2]

2-(1-isopropyl-4-methyl-1H-pyrazol-5-yl)-4-(methylthio)-7,8-dihydroquinazolin-5(6H)-one (500 mg, 1.6 mmol) was dissolved to anhydrous ethanol (10 mL), added with 98% hydrazine hydrate (180 mg, 3.5 mmol), and reacted at 70° C. for 7 h. After the reaction, the reaction mixture was concentrated to give crude product directly used for next reaction.

(3) Preparation of 4-(1-isopropyl-4-methyl-1H-pyrazol-5-yl)-2,6,7,8-tetrahydropyrazolo[3,4,5]quinazoline

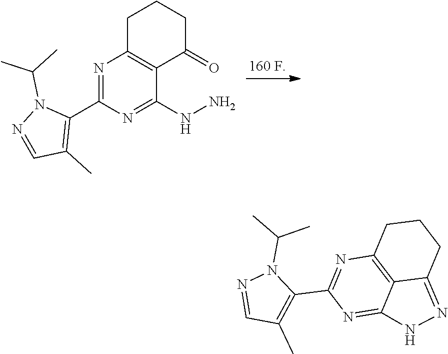 [SMILES: Cc1cnn(C(C)C)c1-c1nc2c(c(NN)n1)C(=O)CCC2 ; Cc1cnn(C(C)C)c1-c1nc2c3c(n[nH]c3n1)CCC2]

The crude product from the above step was dissolved to NMP (10 mL), added with p-toluenesulfonic acid (50 mg), and the system reacted at 160° C. for 1.5 h with microwaves. After the reaction, the reaction mixture was washed with water, extracted with ethyl acetate, subjected to solution separation and concentrated, and purified by silica gel column chromatography (normal heptane: ethyl acetate=1:1) to give 399 mg of the target product with a two-step yield of 89.4%.

(4) Preparation of 4-(1-isopropyl-4-methyl-1H-pyrazol-5-yl)-2-(4-(1-methyl-4-(trifluoromethyl)-1H-imidazol-2-yl)benzyl)-2,6,7,8-tetrahydropyrazolo[3,4,5-de]quinazoline

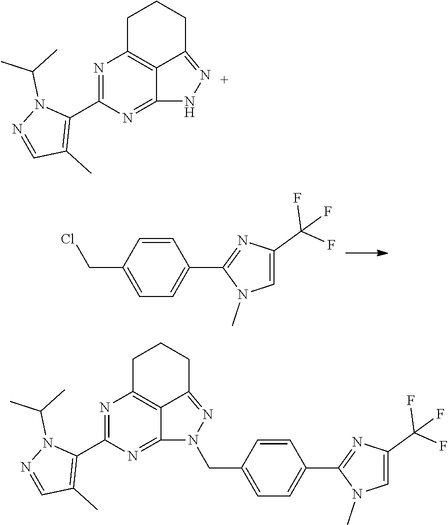 [SMILES: Cc1cnn(C(C)C)c1-c1nc2c3c(n[nH]c3n1)CCC2 ; Cn1cc(C(F)(F)F)nc1-c1ccc(CCl)cc1 ; Cc1cnn(C(C)C)c1-c1nc2c3c(nn(Cc4ccc(-c5nc(C(F)(F)F)cn5C)cc4)c3n1)CCC2]

4-(1-isopropyl-4-methyl-1H-pyrazol-5-yl)-2,6,7,8-tetrahydropyrazolo[3,4,5-de]quinazoline (150 mg, 0.53 mmol), 2-(4-(chloromethyl)phenyl)-1-methyl-4-(trifluoromethyl)-1H-imidazole (218 mg, 0.79 mmol), cesium carbonate (518 mg, 1.6 mmol) and DMF (10 mL) were weighed in turn, and reacted at 50° C. for 13 h. After LC-MS showed that the reaction was completed, the reaction solution was diluted with water, extracted with EA. Organic phases were spin-dried, and the residue was separated by Prep-TLC (SiO₂, PE:EA=1:2, Rf=0.5), and then separated by reversed column chromatography (C_{1-8,} H₂O:MeOH=90:10 to 65:35) to give 150 mg of the product with a yield of 54.2%.

Molecular formula: C₂₇H₂₇F₃N₈ Molecular weight: 520.6 LC-MS (m/z): 521.3 (M+H⁺)

¹H-NMR(400 MHz, CDCl₃): 7.52 (d, J=8.4, 2H), 7.61 (d, J=8.4, 2H), 7.47 (s, 1H), 7.30 (s, 1H), 5.65 (s, 2H), 5.44-5.37 (m, 1H), 3.75 (s, 3H), 3.11-3.07 (m, 4H), 2.43-2.36 (m, 2H), 2.32 (s, 3H), 1.56-1.53 (m, 6H).

Example 8 Preparation of 2-(4-(1-isopropyl-4-(trifluoromethyl)-1H-imidazol-2-yl)benzyl)-4-(1-isopropyl-4-methyl-1H-pyrazol-5-yl)-2,6,7,8- tetrahydropyrazolo[3,4,5-de]quinazoline (Compound 19)

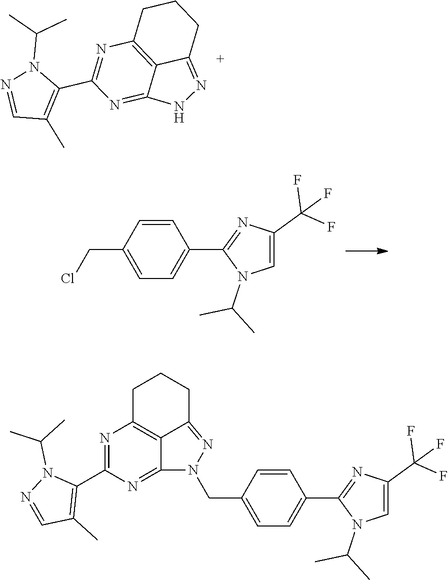 [SMILES: Cc1cnn(C(C)C)c1-c1nc2c3c(n[nH]c3n1)CCC2 ; CC(C)n1cc(C(F)(F)F)nc1-c1ccc(CCl)cc1 ; Cc1cnn(C(C)C)c1-c1nc2c3c(nn(Cc4ccc(-c5nc(C(F)(F)F)cn5C(C)C)cc4)c3n1)CCC2]

4-(1-isopropyl-4-methyl-1H-pyrazol-5-yl)-2,6,7,8-tetrahydropyrazolo[3,4,5-de]quinazoline (120 mg, 0.42 mmol) was dissolved to DMF (5 mL), and added with 2-(4-(chloromethyl)phenyl)-1-isopropyl-4-(trifluoromethyl)-1H-imidazole (120 mg, 0.40 mmol) and potassium carbonate (150 mg, 1.1 mmol). The reaction mixture reacted at 50° C. for 2 h. After the reaction, the reaction mixture was filtered, concentrated and purified by silica gel plate (ethyl acetate: normal heptane=2:1) to give 23 mg of the target product with a yield of 10.5%.

Molecular formula: C₂₉H₃₁F₃N₈ Molecular weight: 548.6 LC-MS (M/e): 549.4(M+H⁺)

¹H-NMR (400 MHz, CDCl₃) δ:7.92-7.69(m, 4H), 7.47-7.28 (m,2H), 5.89 (s, 2H), 5.41 (s, 1H), 4.54 (s,1H), 3.19-3.01 (m,4H), 2.5 (s,2H), 2.40 (s,3H), 1.72-1.5 (m, 6H), 1.55-1.43 (m, 6H).

Example 9 Preparation of 4-(4-cyclopropyl-6-(difluoromethoxy)pyrimidin-5-yl)-2-(4-(1-methyl-4-(trifluoromethyl)-1H-imidazol-2-yl)benzyl)-2,6,7,8- tetrahydropyrazolo[3,4,5-de]quinazoline (Compound 20)

(1) Preparation of 2-(bis(methylthio)methylene)cyclohexane-1,3-dione

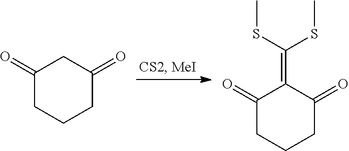

1-3-cyclohexanedione (12.5 g, 0.11 mol) was dissolved to DMF (100 mL), added with potassium carbonate (45.6 g, 0.33 mol) and reacted at 20° C. for 0.5 h. Carbon disulphid (12.9 g, 0.17 mol) was added and reacted at 20° C. for 1 h. Methyl iodide (46.8 g, 0.33 mol) was added and reacted at 20° C. for 1 h. The reaction mixture was concentrated to give the product used directly in next step.

(2) Preparation of 2,4-bis(methylthio)-7,8-dihydroquinazolin-5(6H)-one

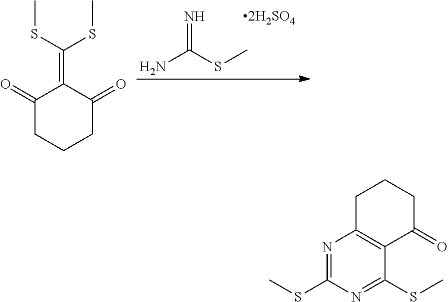

2-(bis(methylthio)methylene)cyclohexane-1,3-dione (crude product from the above step) was dissolved to DMF (200 mL), added with methylisothiourea disulfate (11.5 g, 40.1 mmol) and potassium carbonate (18.5 g, 133.8 mmol), reacted at 100° C. for 16 h, cooled to 25° C., added with water (300 mL), filtered to obtain a filter cake. The filter cake was dried in vacuum to give 2.48 g of the product with a yield of 26%.

(3) Preparation of 4-hydrazino-2-(methylthio)-7,8-dihydroquinazolin-5(6H)-one

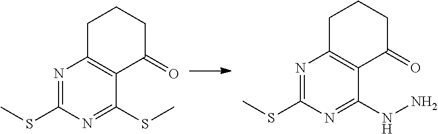

2,4-bis(methylthio)-7,8-dihydroquinazolin-5(6H)-one (2.0 g, 8.3 mmol) was dissolved to ethanol (20 mL), added with hydrazine hydrate (1.0 g, 20.0 mmol), and reacted at 70° C. for 3 h. After the reaction, the reaction mixture was concentrated to give crude product.

(4) Preparation of 4-(methylthio)-2,6,7,8-tetrahydropyrazolo[3,4,5-de]quinazoline

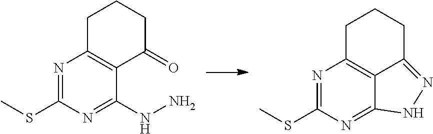

4-hydrazino-2-(methylthio)-7,8-dihydroquinazolin-5(6H)-one (crude product from the above step) was dissolved to DMAc (20 ml), added with p-toluenesulfonic acid (0.25 g, 1.5 mmol), stirred and heated to 160° C., and reacted for 2 h. Then, the reaction was stopped, and the DMAc was spin-evaporated to give the crude product, which was separated by normal-phase chromatographic column (petroleum ether:ethyl acetate=10:0 to 4:6) to give 0.45 g of the product with a yield of 26.3% (two-step).

(5) Preparation of 2-(4-(1-methyl-4-(trifluoromethyl)-1H-imidazol-2-yl)benzyl)-4-(methylthio)-2,6,7,8-tetrahydropyrazolo[3,4,5-de]quinazoline

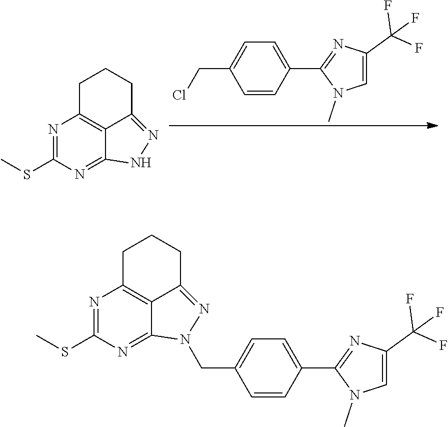 [SMILES: CSc1nc2c3c(n[nH]c3n1)CCC2 ; Cn1cc(C(F)(F)F)nc1-c1ccc(CCl)cc1 ; CSc1nc2c3c(nn(Cc4ccc(-c5nc(C(F)(F)F)cn5C)cc4)c3n1)CCC2]

4-(methylthio)-2,6,7,8-tetrahydropyrazolo[3,4,5-de]quinazoline (206 mg, 1.0 mmol), 2-(4-(chloromethyl)phenyl)-1-methyl-4-(trifluoromethyl)-1H-imidazole (411 mg, 1.5 mmol) and potassium carbonate (414 mg, 3.0 mmol) were added into DMF (20 ml), and heated to 80° C. After 4 hours of reaction, the reaction was stopped, the reaction mixture was filtered, and the filtrate was spin-evaporated to give a crude product, which was separated by normal phase column (petroleum ether:ethyl acetate=10:0 to 4:6) to give 200 mg of the product with a yield of 45%.

(6) Preparation of 2-(4-(1-methyl-4-(trifluoromethyl)-1H-imidazol-2-yl)benzyl)-4-(methylsulfonyl)-2,6,7,8-tetrahydropyrazolo[3,4,5-de]quinazoline

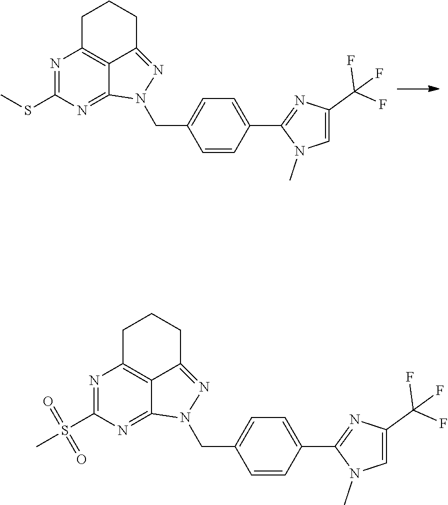 [SMILES: CSc1nc2c3c(nn(Cc4ccc(-c5nc(C(F)(F)F)cn5C)cc4)c3n1)CCC2 ; Cn1cc(C(F)(F)F)nc1-c1ccc(Cn2nc3c4c(nc(S(C)(=O)=O)nc42)CCC3)cc1]

2-(4-(1-methyl-4-(trifluoromethyl)-1H-imidazol-2-yl)benzyl)-4-(methylthio)-2,6,7,8-tetrahydropyrazolo[3,4,5-de]quinazoline (200 mg, 0.45 mmol) was dissolved to dichloromethane (10 mL), added with metachloroperbenzoic acid (155 mg, 0.90 mmol), reacted at 20° C. for 1 h. Then, the reaction was stopped, and the reaction mixture was added with 50 ml of dichloromethane, 50 ml of saturated aqueous sodium carbonate solution and stirred for 5 min, stood for solution separation, and organic phases were collected and spin-evaporated to give a crude product, which was directly used in the next step.

(7) Preparation of 2-(4-(1-methyl-4-(trifluoromethyl)-1H-imidazol-2-yl)benzyl)-2,6,7,8-tetrahydropyrazolo[3,4,5-de]quinazoline-4-ol

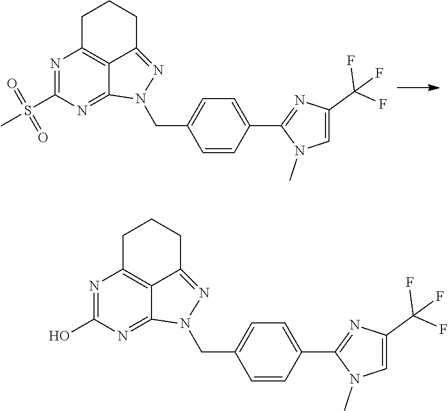 [SMILES: Cn1cc(C(F)(F)F)nc1-c1ccc(Cn2nc3c4c(nc(S(C)(=O)=O)nc42)CCC3)cc1 ; Cn1cc(C(F)(F)F)nc1-c1ccc(Cn2nc3c4c(nc(O)nc42)CCC3)cc1]

2-(4-(1-methyl-4-(trifluoromethyl)-1H-imidazol-2-yl)benzyl)-4-(methylsulfonyl)-2,6,7,8-tetrahydropyrazolo[3,4,5-de]quinazoline (crude product from the above step) was dissolved to tetrahydrofuran (20 mL), added with 10 ml of 10% KOH aqueous solution, stirred at 20° C. for 4 h, then the reaction was stopped, and a pH was adjusted to 4-5 by adding 1M HCl. The solvent in the reaction solution was spin-evaporated, and 200 ml of mixed solvent of dichloromethane and methanol (10:1) was added to elute the product, which was spin-evaporated to give a crude product. 2 ml of ethyl acetate was added into the reaction solution, stirred evenly, and filtered to give 200 mg of the product (crude product).

(8) Preparation of 4-chloro-2-(4-(1-methyl-4-(trifluoromethyl)-1H-imidazol-2-yl)benzyl)-2,6,7,8-tetrahydropyrazolo[3,4,5-de]quinazoline

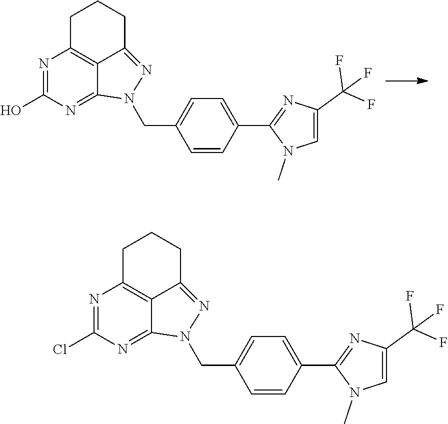 [SMILES: Cn1cc(C(F)(F)F)nc1-c1ccc(Cn2nc3c4c(nc(O)nc42)CCC3)cc1 ; Cn1cc(C(F)(F)F)nc1-c1ccc(Cn2nc3c4c(nc(Cl)nc42)CCC3)cc1]

2-(4-(1-methyl-4-(trifluoromethyl)-1H-imidazol-2-yl)benzyl)-2,6,7,8-tetrahydropyrazolo[3,4,5-de]quinazoline-4-ol (200 mg of crude product) was dissolved to phosphorus oxychloride (6 mL), reacted at 110° C. for 4 h. LCMS showed that the reaction was completed. The reaction solution was spin-dried, the residue was diluted with saturated sodium bicarbonate solution, and extracted with EA. Organic phases were spin-dried, and the residue was separated by column chromatography (SiO₂, PE:EA=4:1 to 2:3) to give 100 mg of the product.

(9) Preparation of 4-(4-cyclopropyl-6-(difluoromethoxy)pyrimidin-5-yl)-2-(4-(1-methyl-4-(trifluoromethyl)-1H-imidazol-2-yl)benzyl)-2,6,7,8- tetrahydropyrazolo[3,4,5-de]quinazoline

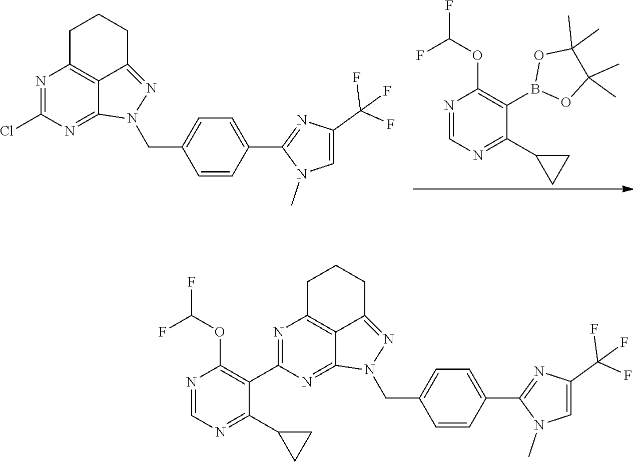 [SMILES: Cn1cc(C(F)(F)F)nc1-c1ccc(Cn2nc3c4c(nc(Cl)nc42)CCC3)cc1 ; CC1(C)OB(c2c(OC(F)F)ncnc2C2CC2)OC1(C)C ; Cn1cc(C(F)(F)F)nc1-c1ccc(Cn2nc3c4c(nc(-c5c(OC(F)F)ncnc5C5CC5)nc42)CCC3)cc1]

4-chloro-2-(4-(1-methyl-4-(trifluoromethyl)-1H-imidazol-2-yl)benzyl)-2,6,7,8-tetrahydropyrazolo[3,4,5-de]quinazoline (90 mg, 021 mmol), 4-cyclopropyl-6-(difluoromethoxy)-5-(4,4,5,5-tetramethyl-1,3,2-dioxobenzofuran-2-yl)pyrimidine (330 mg of crude product), XPhos-Pd-G2 (17 mg, 0.022 mmol), XPhos (10 mg, 0.021 mmol), potassium phosphate (134 mg, 0.63 mmol), dioxane (5 mL) and water (1 mL) were weighed, and then reacted at 90° C. for 2 h. LCMS showed that the reaction was completed, the reaction solution was spin-dried, and the residue was separated by column chromatography (SiO₂, PE:EA=4:1 to 2:3) to give 40 mg of the product with a yield of 33.0%.

Molecular formula: C₂₈H₂₃F₅N₈O Molecular weight: 582.5 LC-MS (m/z):583.1 (M+H⁺)

¹H-NMR(400 MHz, CDCl₃) δ: 8.66 (s, 1H), 7.72-7.20 (m, 6H), 5.64 (s, 2H), 3.72 (s, 3H), 3.15-3.05 (m, 4H), 2.45-2.35 (m, 2H), 1.85-1.75 (m, 1H), 1.30-1.20 (m, 2H), 1.05-0.95 (m, 2H).

Example 10 Preparation of 4-(4-cyclopropyl-6-(methoxy-d₃)pyrimidin-5-yl)-2-(4-(1-methyl-4-(trifluoromethyl)-1H-imidazol-2-yl)benzyl)-2,6,7,8- tetrahydropyrazolo[3,4,5-de]quinazoline (Compound 1-1)

(1) Preparation of 2-(4-cyclopropyl-6-(methoxy-d₃)pyrimidin-5-yl)-4-(methylthio)-7,8-dihydroquinazolin-5(6H)-one

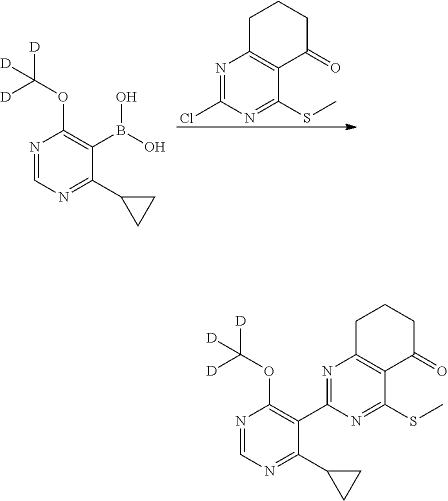 [SMILES: [2H]C([2H])([2H])Oc1ncnc(C2CC2)c1B(O)O ; CSc1nc(Cl)nc2c1C(=O)CCC2 ; [2H]C([2H])([2H])Oc1ncnc(C2CC2)c1-c1nc2c(c(SC)n1)C(=O)CCC2]

2-chloro-4-(methylthio)-7,8-dihydroquinazolin-5(6H)-one (1.2 g, 5.2 mmol) was dissolved to 1,4-dioxane (30 mL), added with (4-cyclopropyl-6-(methoxy-d₃)pyrimidin-5-yl)boronic acid (1 g, 5.1 mmol), Xphos-Pd-G2 (408 mg, 0.52 mmol), Xphos (496 mg, 1.04 mmol), K₃PO₄ (1.2 g, 5.7 mmol) and water (10 mL), and reacted at 90° C. for 3 h under the protection of N₂. After the reaction, the solvent was spin-dried to give 600 mg of the product by normal phase preparation and separation (ethyl acetate/petroleum ether=0-50%) with a yield of 34.1%.

(2) Preparation of 2-(4-cyclopropyl-6-(methoxy-d₃)pyrimidin-5-yl)-4-hydrazino-7,8-dihydroquinazolin-5(6H)-one

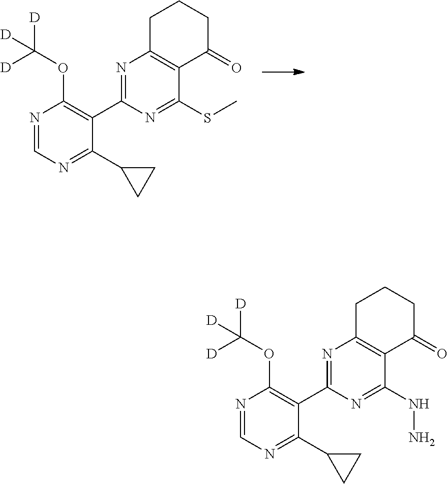 [SMILES: [2H]C([2H])([2H])Oc1ncnc(C2CC2)c1-c1nc2c(c(SC)n1)C(=O)CCC2 ; [2H]C([2H])([2H])Oc1ncnc(C2CC2)c1-c1nc2c(c(NN)n1)C(=O)CCC2]

2-(4-cyclopropyl-6-(methoxy-d₃)pyrimidin-5-yl)-4-(methylthio)-7,8-dihydroquinazolin-5(6H)-one (500 mg, 1.4 mmol) was dissolved to ethanol (10 mL), added with hydrazine hydrate (87 mg, 1.7 mmol), and reacted at 70° C. for 3 h. After the reaction, the reaction mixture was concentrated to give 400 mg of the product with a yielding of 86.8%.

(3) Preparation of 4-(4-cyclopropyl-6-(methoxy-d₃)pyrimidin-5-yl)-2,6,7,8-tetrahydropyrazolo[3,4,5-de]quinazoline

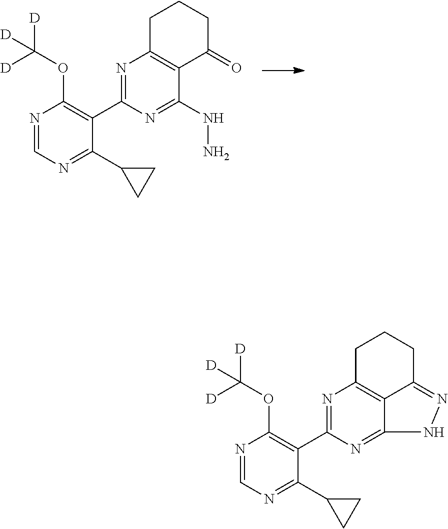 [SMILES: [2H]C([2H])([2H])Oc1ncnc(C2CC2)c1-c1nc2c(c(NN)n1)C(=O)CCC2 ; [2H]C([2H])([2H])Oc1ncnc(C2CC2)c1-c1nc2c3c(n[nH]c3n1)CCC2]

2-(4-cyclopropyl-6-(methoxy-d₃)pyrimidin-5-yl)-4-hydrazino-7,8-dihydroquinazolin-5(6H)-one (300 mg, 0.92 mmol) was dissolved to NMP (5 mL) and ethanol (60 mL), and reacted at 160° C. for 1 h with microwaves. 150 mg of the product was given by column chromatography separation (methanol/water=0-60%) with a yield of 53%.

(4) Preparation of 4-(4-cyclopropyl-6-(methoxy-d₃)pyrimidin-5-yl)-2-(4-(1-methyl-4-(trifluoromethyl)-1H-imidazol-2-yl)benzyl)-2,6,7,8- tetrahydropyrazolo[3,4,5-de]quinazoline

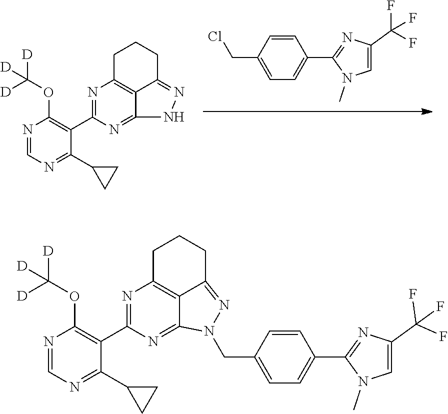 [SMILES: [2H]C([2H])([2H])Oc1ncnc(C2CC2)c1-c1nc2c3c(n[nH]c3n1)CCC2 ; Cn1cc(C(F)(F)F)nc1-c1ccc(CCl)cc1 ; [2H]C([2H])([2H])Oc1ncnc(C2CC2)c1-c1nc2c3c(nn(Cc4ccc(-c5nc(C(F)(F)F)cn5C)cc4)c3n1)CCC2]

4-(4-cyclopropyl-6-(methoxy-d₃)pyrimidin-5-yl)-2,6,7,8-tetrahydropyrazolo[3,4,5-de]quinazoline (100 mg, 0.32 mmol) was dissolved to DMF (4 mL), added with potassium carbonate (88 mg, 0.64 mmol) and 2-(4-(chloromethyl)phenyl)-1-methyl-4-(trifluoromethyl)-1H-imidazole (88 mg, 0.32 mmol), and reacted at 20° C. for 3 h. After the reaction, 42 mg of the product was given by column chromatography separation (methanol/water=0-40%) with a yield of 24%.

Molecular formula: C₂₈H₂₂D₃F₃N₈O Molecular weight: 549.58 LC-MS (M/e): 550.2(M+H⁺) ¹H-NMR (400 MHz, DMSO) δ: 8.68(s, 1H), 7.92(s, 1H), 7.76-7.68 (m, 2H), 7.44-7.42 (m, 2H), 5.79(s, 2H), 3.59(s,3H), 3.05-3.00 (m, 4H), 2.45-2.32 (m, 2H), 1.69-1.55 (m, 1H), 1.25-1.19 (m, 2H), 0.92-0.75 (m, 2H).

Example 11 Preparation of 4-(4-cyclopropyl-6-(methoxy-d₃)pyrimidin-5-yl)-2-(4-(1-methyl-4-(trifluoromethyl)-1H-imidazol-2-yl)benzyl)-6,7,8,9- tetrahydro-2H-1,2,3,5-tetraazabenzo[cd]azulene (Compound 11-1)

(1) Preparation of 2-(di-tert-butoxycarbonyl)amino-4-(methylthio)-6,7,8,9-tetrahydro-5H-cyclohepta[d]pyrimidin-5-one

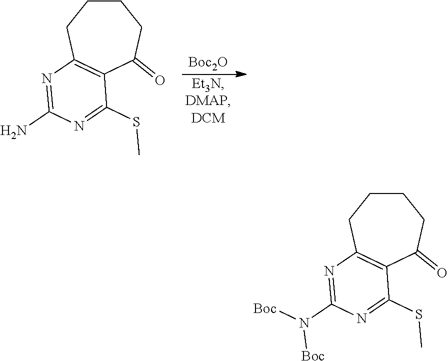

A DCM (60 mL) solution of 2-amino-4-(methylthio)-6,7,8,9-tetrahydro-5H-cyclohepta[d]pyrimidin-5-one (2.0 g, 9.0 mmol) was added with triethylamine (4.6 g, 45.5 mmol), p-dimethylaminopyridine (110 mg, 0.9 mmol) and Boc₂O (7.9 g, 36.2 mmol) in turn, and reacted at 25° C. for 1 h. LC-MS showed that the reaction was completed, the reaction solution was spin-dried and the residue was separated by column chromatography (SiO₂, PE:EA=10:1 to 2:1) to give 3.4 g of the product with a yield of 89.6%.

(2) Preparation of tert-butyl(6,7,8,9-tetrahydro-2H-1,2,3,5-tetraazabenzo[cd]azulene-4-yl)carbamate

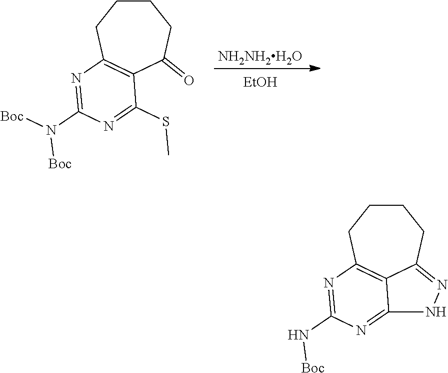 [SMILES: "EtOH" ; CSc1nc(N(C(=O)OC(C)(C)C)C(=O)OC(C)(C)C)nc2c1C(=O)CCCC2 ; "NH2NH2·H2O" ; CC(C)(C)OC(=O)Nc1nc2c3c(n[nH]c3n1)CCCC2]

An ethanol (50 mL) solution of 2-(di-tert-butoxycarbonyl)amino-4-(methylthio)-6,7,8,9-tetrahydro-5H-cyclohepta[d]pyrimidin-5-one (3.2 g, 7.6 mmol) was added with hydrazine hydrate (1.9 g, 37.9 mmol), and reacted at 80° C. for 13 h. LC-MS showed that the reaction was completed, the reaction solution was spin-dried and the residue was separated by column chromatography (SiO₂, DCM:MeOH=50:1 to 10:1) to give 1.5 g of the product with a yield of 68.6%.

(3) Preparation of tert-butyl(2-(4-(1-methyl-4-(trifluoromethyl)-1H-imidazol-2-yl)benzyl)-6,7,8,9-tetrahydro-2H-1,2,3,5-tetraazabenzo[cd]azulene-4- yl)carbamate

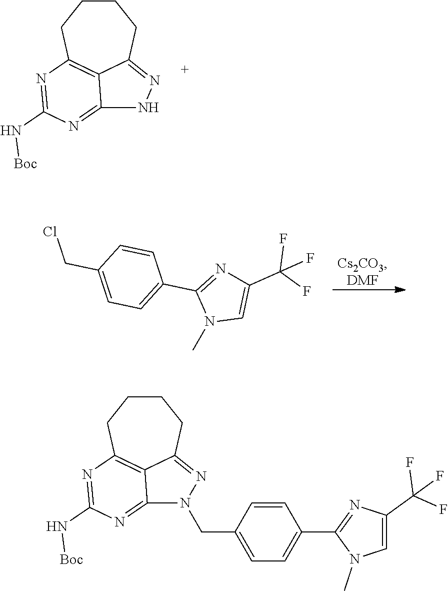

Tert-butyl(6,7,8,9-tetrahydro-2H-1,2,3,5-tetraazabenzo[cd] azulene-4-yl)carbamate (1.4 g, 4.8 mmol), 2-(4-(chloromethyl)phenyl)-1-methyl-4-(trifluoromethyl)-1H-imidazole (1.6 g, 5.8 mmol), cesium carbonate (3.1 g, 9.5 mmol) and DMF (30 mL) were weighed in turn, and reacted at 70° C. for 6 h. LC-MS showed that the reaction was completed, the reaction solution was diluted with water, and extracted with ethyl acetate. Organic phases were dried, and spin-dried. The residue was separated by column chromatography (SiO₂, PE:EA=2:1 to 1:1) to give 1.9 g of the product with a yield of 74.4%.

(4) Preparation of 2-(4-(1-methyl-4-(trifluoromethyl)-1H-imidazol-2-yl)benzyl)-6,7,8,9-tetrahydro-2H-1,2,3,5-tetraazabenzo[cd]azulene-4-amine

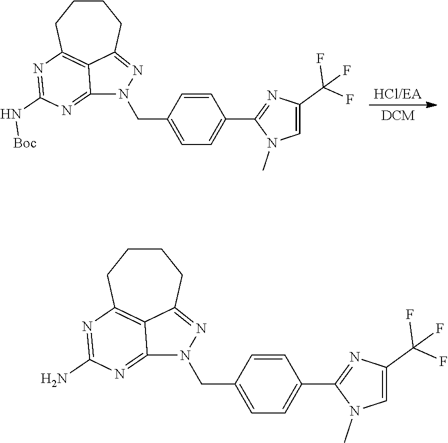

A DCM (10 mL) solution of tert-butyl(2-(4-(1-methyl-4-(trifluoromethyl)-1H-imidazol-2-yl)benzyl)-6,7,8,9-tetrahydro-2H-1,2,3,5-tetraazabenzo[cd]azulene-4- yl)carbamate (1.9 g, 3.6 mmol) was added with HCl/EA (20 mL, 80.0 mmol) and reacted at 25° C. for 2 h. LC-MS showed that the reaction was completed, the reaction solution was diluted with water, adjusted with ammonia water till a pH was 9, and then extracted with DCM. Organic phases were dried, and spin-dried. The residue was separated by column chromatography (SiO₂, DCM:MeOH=50:1 to 30:1) to give 1.2 g of the product with a yield of 77.9%.

(5) Preparation of 4-chloro-2-(4-(1-methyl-4-(trifluoromethyl)-1H-imidazol-2-yl)benzyl)-6,7,8,9-tetrahydro-2H-1,2,3,5-tetraazabenzo[cd]azulene

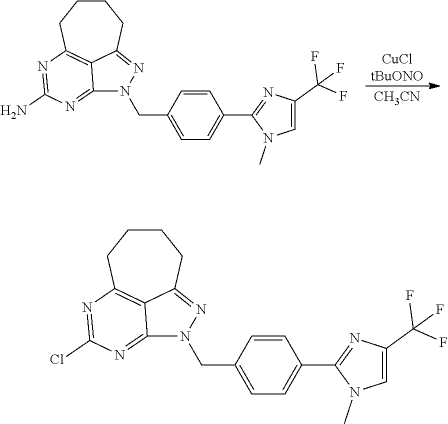

At 0° C., an acetonitrile (3 mL) solution of 2-(4-(1-methyl-4-(trifluoromethyl)-1H-imidazol-2-yl)benzyl)-6,7,8,9-tetrahydro-2H-1,2,3,5-tetraazabenzo[cd]azulene-4-amine (500 mg, 1.2 mmol) was added with cuprous chloride (238 mg, 2.4 mmol) and tert-butyl nitrite (247 mg, 2.4 mmol), and reacted at 60° C. for 4 h. LC-MS showed that the reaction was completed, the reaction solution was filtered, and the filtrate was spin-dried. The residue was separated by column chromatography (SiO₂, DCM:MeOH=50:1 to 30:1) to give 100 mg of the product with a yield of 19.1%.

(6) Preparation of 4-(4-cyclopropyl-6-(methoxy-d₃)pyrimidin-5-yl)-2-(4-(1-methyl-4-(trifluoromethyl)-1H-imidazol-2-yl)benzyl)-6,7,8,9-tetrahydro-2H-1,2,3,5-tetraazabenzo[cd]azulene

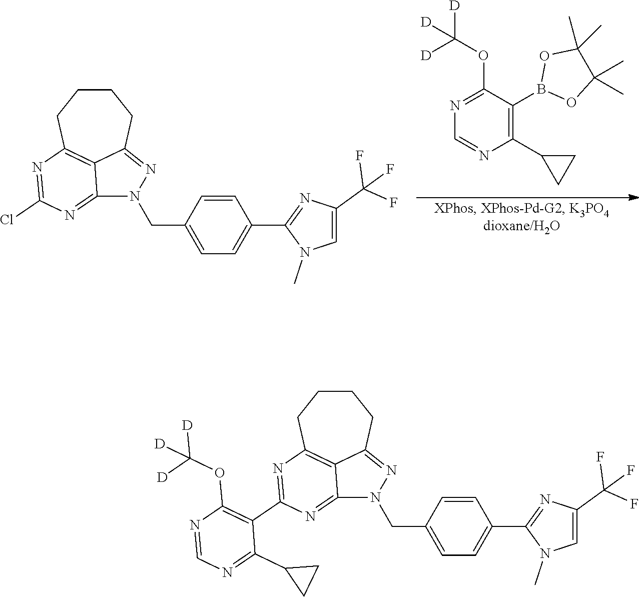 [SMILES: Cn1cc(C(F)(F)F)nc1-c1ccc(Cn2nc3c4c(nc(Cl)nc42)CCCC3)cc1 ; [2H]C([2H])([2H])Oc1ncnc(C2CC2)c1B1OC(C)(C)C(C)(C)O1 ; "XPhos, XPhos-Pd-G2, K3PO4dioxane/H2O" ; [2H]C([2H])([2H])Oc1ncnc(C2CC2)c1-c1nc2c3c(nn(Cc4ccc(-c5nc(C(F)(F)F)cn5C)cc4)c3n1)CCCC2]

4-chloro-2-(4-(1-methyl-4-(trifluoromethyl)-1H-imidazol-2-yl)benzyl)-6,7,8,9-tetrahydro-2H-1,2,3,5-tetraazabenzo[cd]azulene (100 mg, 0.22 mmol), 4-cyclopropyl-6-(methoxy-d₃)-5-(4,4,5,5-tetramethyl-1,3,2-dioxobenzaldehyde-2-yl)pyrimidine (50 mg of crude product), XPhos (10 mg, 0.021 mmol), XPhos-Pd-G2 (17 mg, 0.022 mmol), potassium phosphate (140 mg, 0.66 mmol), dioxane (5 mL) and water (1 mL) were weighed in turn, subjected to nitrogen replacement for 3 times, and reacted at 90° C. for 6 h. LC-MS showed that the reaction was completed, the reaction solution was filtered, and the filtrate was spin-dried. The residue was separated by thin layer chromatography (SiO₂, DCM:MeOH=30:1, Rf-0.4) to give 16 mg of the product with a yield of 12.7%.

Molecular formula: C₂₉H₂₄D₃F₃N₈O Molecular weight: 563.6 LC-MS (m/z): 564.2 (M+H⁺)

¹H-NMR(400 MHz, CDCl₃) δ: 8.68 (s, 1H), 7.59 (d, 2H, J=8.0), 7.53 (d, 2H, J=8.0), 7.31 (s, 1H), 5.69 (s, 2H), 3.75 (s, 3H), 3.43-3.33 (m, 2H), 3.24-3.14 (m, 2H), 2.33-2.12 (m, 5H), 1.20-0.90 (m, 4H).

Example 12 Preparation of 4-(4-cyclopropyl-6-(methoxy-d₃)pyrimidin-5-yl)-2-(4-(1-isopropyl-4-(trifluoromethyl)-1H-imidazol-2-yl)benzyl)-2,6,7,8- tetrahydropyrazolo[3,4,5-de]quinazoline (Compound 16-1)

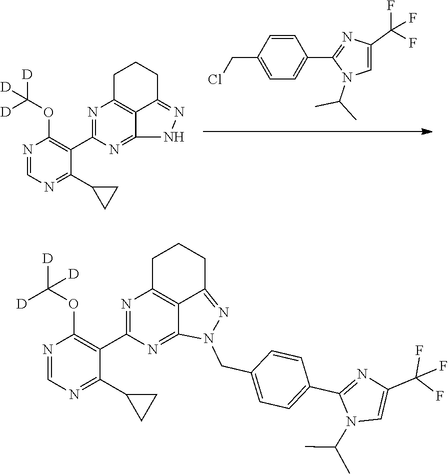 [SMILES: [2H]C([2H])([2H])Oc1ncnc(C2CC2)c1-c1nc2c3c(n[nH]c3n1)CCC2 ; CC(C)n1cc(C(F)(F)F)nc1-c1ccc(CCl)cc1 ; [2H]C([2H])([2H])Oc1ncnc(C2CC2)c1-c1nc2c3c(nn(Cc4ccc(-c5nc(C(F)(F)F)cn5C(C)C)cc4)c3n1)CCC2]

4-(4-cyclopropyl-6-(methoxy-d₃)pyrimidin-5-yl)-2,6,7,8-tetrahydropyrazolo[3,4,5-de]quinazoline (93 mg, 0.3 mmol) was dissolved to DMF (5 mL), added with potassium carbonate (83 mg, 0.6 mmol) and 2-(4-(chloromethyl)phenyl)-1-isopropyl-4-(trifluoromethyl)-1H-imidazole (100 mg, 0.3 mmol), and reacted at 70° C. for 2 h. After the reaction, the reaction mixture was subjected to column chromatography separation (PE/EA=3:1 to 1:1) to give 43 mg of the product with a yield of 24.2%.

Molecular formula: C₃₀H₂₆D₃F₃N₈O Molecular weight: 577.6 LC-MS (M/e): 578.3(M+H⁺)

¹H-NMR (400 MHz, DMSO) δ: 8.69(s, 1H), 8.16(s, 1H), 7.55-7.49 (d, 2H), 7.44-7.39 (d, 2H), 5.65(s, 2H), 4.45-4.35(m,1H), 3.09-3.00 (m, 4H), 2.30-2.22 (m, 2H), 1.65-1.55 (m, 1H), 1.38-1.32 (m, 6H), 1.09-1.01 (m, 2H), 0.86-0.80 (m, 2H).

Example 13 Preparation of 4-(4-cyclopropyl-6-(methoxy-d₃)pyrimidin-5-yl)-2-(4-(1-isopropyl-4-(trifluoromethyl)-1H-imidazol-2-yl)benzyl)-6,7,8,9- tetrahydro-2H-1,2,3,5-tetraazabenzo[cd]azulene(Compound 17-1)

(1) Preparation of 2-(4-cyclopropyl-6-(methoxy-d₃)pyrimidin-5-yl)-4-(methylthio)-6,7,8,9-tetrahydro-5H-cyclohepta[d]pyrimidin-5-one

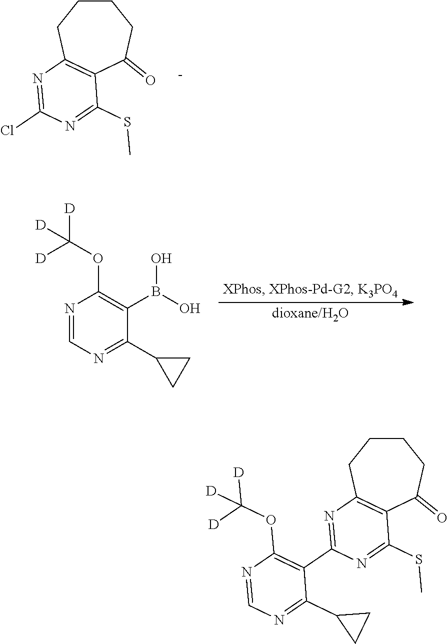 [SMILES: CSc1nc(Cl)nc2c1C(=O)CCCC2 ; [2H]C([2H])([2H])Oc1ncnc(C2CC2)c1B(O)O ; "XPhos, XPhos-Pd-G2, K3PO4" ; "dioxane/H2O" ; [2H]C([2H])([2H])Oc1ncnc(C2CC2)c1-c1nc2c(c(SC)n1)C(=O)CCCC2]

2-chloro-4-(methylthio)-6,7,8,9-tetrahydro-5H-cyclohepta[d]pyrimidin-5-one (400 mg, 1.6 mmol), (4-cyclopropyl-6-(methoxy-d₃)pyrimidin-5-yl)boronic acid (347 mg, 1.8 mmol), XPhos (76 mg, 0.16 mmol), XPhos-Pd-G2 (126 mg, 0.16 mmol), potassium phosphate (1.0 g, 4.7 mmol), dioxane (20 mL) and water (4 mL) were weighed in turn, subjected to nitrogen replacement for three times, and reacted at 90° C. for 4 h. LC-MS showed that the reaction was completed, the reaction solution was filtered, and the filtrate was spin-dried. The residue was separated by column chromatography (SiO₂, PE:EA=5:1 to 3:1) to give 260 mg of the product with a yield of 43.9%.

(2) Preparation of 4-(4-cyclopropyl-6-(methoxy-d₃)pyrimidin-5-yl)-6,7,8,9-tetrahydro-2H-1,2,3,5-tetraazabenzo[cd]azulene

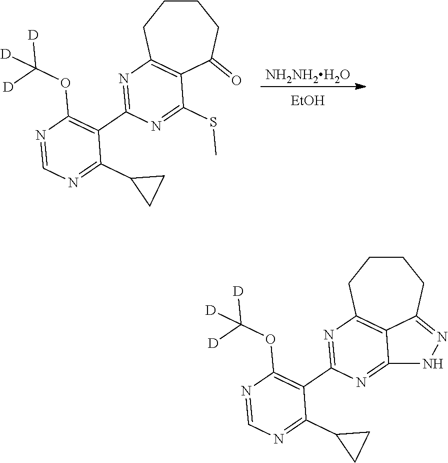 [SMILES: "EtOH" ; [2H]C([2H])([2H])Oc1ncnc(C2CC2)c1-c1nc2c(c(SC)n1)C(=O)CCCC2 ; "NH2NH2·H2O" ; [2H]C([2H])([2H])Oc1ncnc(C2CC2)c1-c1nc2c3c(n[nH]c3n1)CCCC2]

An ethanol (6 mL) solution of 2-(4-cyclopropyl-6-(methoxy-d₃)pyrimidin-5-yl)-4-(methylthio)-6,7,8,9-tetrahydro-5H-cyclohepta[d]pyrimidin-5-one (240 mg, 0.67 mmol) was added with hydrazine hydrate (168 mg, 3.4 mmol), and reacted at 80° C. for 3 h. LC-MS showed that the reaction was completed, the reaction solution was spin-dried and the residue was separated by column chromatography (SiO₂, PE:EA=3:1 to 1:1) to give 200 mg of the product with a yield of 92.0%.

(3) Preparation of 4-(4-cyclopropyl-6-(methoxy-d₃)pyrimidin-5-yl)-2-(4-(1-isopropyl-4-(trifluoromethyl)-1H-imidazol-2-yl)benzyl)-6,7,8,9- tetrahydro-2H-1,2,3,5-tetraazabenzo[cd]azulene

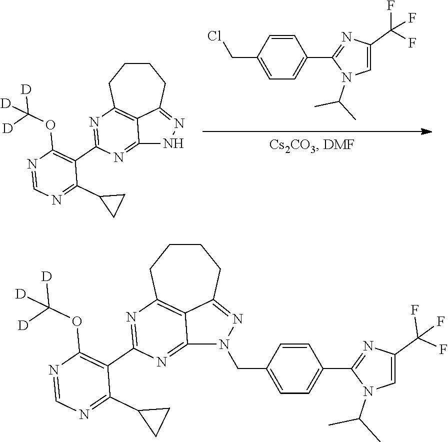 [SMILES: [2H]C([2H])([2H])Oc1ncnc(C2CC2)c1-c1nc2c3c(n[nH]c3n1)CCCC2 ; CC(C)n1cc(C(F)(F)F)nc1-c1ccc(CCl)cc1 ; [2H]C([2H])([2H])Oc1ncnc(C2CC2)c1-c1nc2c3c(nn(Cc4ccc(-c5nc(C(F)(F)F)cn5C(C)C)cc4)c3n1)CCCC2]

A DMF (5 mL) solution of 4-(4-cyclopropyl-6-(methoxy-d₃)pyrimidin-5-yl)-6,7,8,9-tetrahydro-2H-1,2,3,5-tetraazabenzo[cd]chamomile (200 mg, 0.61 mmol) was added with 2-(4-(chloromethyl)phenyl)-1-isopropyl-4-(trifluoromethyl)-1H-imidazole (203 mg, 0.67 mmol) and cesium carbonate (596 mg, 1.8 mmol), and reacted at 50° C. for 13 h. LC-MS showed that the reaction was completed, the reaction solution was diluted with water, and extracted with EA. Organic phases were dried, and spin-dried. The residue was separated by column chromatography (SiO₂, PE:EA=3:1 to 1:1) to give 167 mg of the product with a yield of 45.9%.

Molecular formula: C₃₁H₂₈D₃F₃N₈O Molecular weight: 591.7 LC-MS (m/z): 592.3 (M+H⁺)

¹H-NMR(400 MHz, CDCl₃) δ: 8.67 (s, 1H), 7.52-7.47 (m, 4H), 7.41 (s, 1H), 5.69 (s, 2H), 4.56-4.49 (m, 1H), 3.37-3.34 (m, 2H), 3.19-3.16 (m, 2H), 2.23-2.12 (m, 4H), 1.70-1.62 (m, 1H), 1.45-1.41 (m, 6H), 1.35-1.23 (m, 2H), 0.91-0.86 (m, 2H).

The compounds shown in the following table were prepared by the same or similar methods as in the above examples:

| | LC-MS | | LC-MS |
|---|---|---|---|
| No. | (M/e, M + H⁺) | No. | (M/e, M + H⁺) |
| Compound 4 | 517.2 | Compound 5 | 547.2 |
| Compound 6 | 517.2 | Compound 7 | 533.2 |
| Compound 8 | 533.2 | Compound 9 | 536.2 |
| Compound 12 | 517.2 | Compound 13 | 534.2 |
| Compound 14 | 585.2 | Compound 15 | 597.2 |
| Compound 17 | 590.3 | Compound 5-1 | 550.2 |
| Compound 7-1 | 536.2 | Compound 8-1 | 536.2 |

The USP1 inhibitor and the use thereof provided by the present disclosure are described in detail above. Specific embodiments are used herein to illustrate the principles and embodiments of the present disclosure. The description of the above embodiments is only used to help understand the method and core ideas of the present disclosure. It should be pointed out that for those of ordinary skills in the art, several improvements and modifications can be made to the present disclosure without departing from the principle of the present disclosure, and these improvements and modifications also fall within the protection scope of the claims of the present disclosure.

What is claimed is:

1. A compound of general formula (I) or a pharmaceutically acceptable salt, ester, deuterated compound or stereoisomer thereof, 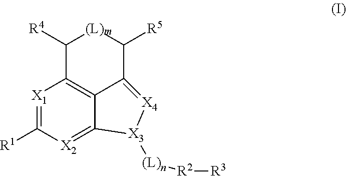
wherein:
X₁, X₂, X₃ and X₄ are each independently selected from N or CR^{a};
R¹, R² and R³ are each independently selected from 3-12 membered cycloalkyl, 3-12 membered heterocyclyl, 6-10 membered aryl or 5-12 membered heteroaryl, each of which is optionally substituted by one or more Q₁;
R⁴ and R⁵ are each independently selected from deuterium, hydrogen, carboxyl, cyano, nitro, amino, halogen, C₂₋₆ alkenyl, C₂₋₆ alkynyl, or from C₁₋₆ alkyl, C₁₋₆ alkoxy, C₁₋₆ alkylamino, di(C₁₋₆ alkyl)amino, halogenated C₁₋₆ alkyl, hydroxy C₁₋₆ alkyl, amino C₁₋₆ alkyl, carboxyl C₁₋₆ alkyl or halogenated C₁₋₆ alkoxy, each of which is optionally deuterated;
each Q₁ is independently selected from deuterium, halogen, cyano, carboxyl, hydroxyl, amino, nitro, sulfonamido, or from C₁₋₆ alkylamino, di(C₁₋₆ alkyl)amino, halogenated C₁₋₆ alkyl, halogenated C₁₋₆ alkoxy, hydroxy C₁₋₆ alkyl, amino C₁₋₆ alkyl, carboxyl C₁₋₆ alkyl, C₁₋₆ alkylcarbonyl, C₁₋₆ alkoxycarbonyl, C₁₋₆ alkylaminoacyl, C₁₋₆ alkylamido, C₁₋₆ alkylsulfonyl,C₁₋₆ alkylsulfonamido, C₁₋₆ alkylaminosulfonyl, —(L)ₘ-C₁₋₆ alkyl, —(L)ₘ-C₂₋₆ alkenyl, —(L)ₘ-C₂₋₆ alkynyl, —(L)ₘ-C₁₋₆ alkoxy, —(L)ₘ-6-10 membered aryl, —(L)ₘ-5-12 membered heteroaryl, —(L)ₘ-3-8 membered cycloalkyl or —(L)ₘ-3-8 membered heterocyclyl, each of which is optionally substituted by 1-4 substituents Q₂; and each Q₂ is independently selected from deuterium, halogen, carboxyl, hydroxyl, cyano, nitro, amino, C₁₋₆ alkyl, hydroxy C₁₋₆ alkyl, carboxyl C₁₋₆ alkyl, C₁₋₆ alkylamino, di(C₁₋₆ alkyl)amino, —CO—C₁₋₆ alkylene-NH₂—, —CO—C₁₋₆ alkyl, C₁₋₆ alkoxy, halogenated C₁₋₆ alkyl and halogenated C₁₋₆ alkoxy;
each L is independently selected from —CO—, —O—, —S—, —SO—, —S(O)₂—, —NR^{c}— or —CR^{a}R^{b}—;
each R^{a} and each R^{b} are each independently selected from deuterium, hydrogen, halogen, amino, hydroxyl, carboxyl, cyano, C₂₋₆ alkenyl, C₂₋₆ alkynyl, or from C₁₋₆ alkyl, C₁₋₆ alkoxy, C₁₋₆ alkylamino, di(C₁₋₆ alkyl)amino, C₁₋₆ alkylaminoacyl, C₁₋₆ alkylamido, C₁₋₆ alkylsulfonamido, C₁₋₆ alkylaminosulfonyl, halogenated C₁₋₆ alkyl, halogenated C₁₋₆ alkoxy, hydroxy C₁₋₆ alkyl, amino C₁₋₆ alkyl and carboxyl C₁₋₆ alkyl, each of which is optionally deuterated;
each R^{c} is independently selected from deuterium, hydrogen, or from C₁₋₆ alkyl, halogenated C₁₋₆ alkyl, halogenated C₁₋₆ alkoxy, hydroxy C₁₋₆ alkyl, amino C₁₋₆ alkyl and carboxyl C₁₋₆ alkyl, each of which is optionally deuterated;
each m is independently an integer of 0-3; and
each n is independently an integer of 0-6.

2. The compound or the pharmaceutically acceptable salt, ester, deuterated compound or stereoisomer thereof according to claim 1,
wherein:
X₁, X₂, X₃ and X₄ are each independently selected from N or CR^{a};
R¹, R² and R³ are each independently selected from 5-8 membered cycloalkyl, 3-8 membered heterocyclyl, phenyl or 5-6 membered heteroaryl, each of which is optionally substituted by one or more Q₁;
R⁴ and R⁵ are each independently selected from deuterium, hydrogen, cyano, halogen, or from C₁₋₆ alkyl, C₁₋₆ alkoxy, C₁₋₆ alkylamino, di(C₁₋₆ alkyl)amino, halogenated C₁₋₆ alkyl, hydroxy C₁₋₆ alkyl, amino C₁₋₆ alkyl, carboxyl C₁₋₆ alkyl or halogenated C₁₋₆ alkoxy, each of which is optionally deuterated;
each Q₁ is independently selected from deuterium, halogen, cyano, or from C₁₋₆ alkoxy, C₁₋₆ alkylamino, di(C₁₋₆ alkyl)amino, halogenated C₁₋₆ alkyl, halogenated C₁₋₆ alkoxy, hydroxy C₁₋₆ alkyl, amino C₁₋₆ alkyl, carboxyl C₁₋₆ alkyl, —(L)ₘ-C₁₋₆ alkyl, —(L)ₘ-3-6 membered cycloalkyl or —(L)ₘ-3-6 membered heterocyclyl, each of which is optionally substituted by 1-4 substituents Q₂; and each Q₂ is independently selected from deuterium, halogen, carboxyl, hydroxyl, cyano, nitro, amino, C₁₋₆ alkyl, hydroxy C₁₋₆ alkyl, carboxyl C₁₋₆ alkyl, C₁₋₆ alkylamino, di(C₁₋₆ alkyl)amino, C₁₋₆ alkoxy, halogenated C₁₋₆ alkyl and halogenated C₁₋₆ alkoxy;
each L is independently selected from —CO—, —O—, —NR^{c}— or —CR^{a}R^{b}—;
each R^{a} and each R^{b} are each independently selected from deuterium, hydrogen, or from C₁₋₆ alkyl, C₁₋₆ alkoxy, C₁₋₆ alkylamino, di(C₁₋₆ alkyl)amino, halogenated C₁₋₆ alkyl, halogenated C₁₋₆ alkoxy, hydroxy C₁₋₆ alkyl, amino C₁₋₆ alkyl and carboxyl C₁₋₆ alkyl, each of which is optionally deuterated;
each R^{c} is independently selected from deuterium, hydrogen, or from C₁₋₆ alkyl, halogenated C₁₋₆ alkyl and halogenated C₁₋₆ alkoxy, each of which is optionally deuterated;
each m is independently an integer of 0-2; and
each n is independently an integer of 0-5.

3. The compound or the pharmaceutically acceptable salt, ester, deuterated compound or stereoisomer thereof according to claim 1,
wherein:
X₁, X₂, X₃ and X₄ are each independently selected from N;
R¹, R² and R³ are each independently selected from phenyl or 5-6 membered heteroaryl optionally substituted by 1-4 Q₁;
R⁴ and R⁵ are each independently selected from deuterium, hydrogen, or optionally deuterated C₁₋₄ alkyl, C₁₋₄ alkoxy, C₁₋₄ alkylamino, di(C₁₋₄ alkyl)amino, halogenated C₁₋₄ alkyl, hydroxy C₁₋₄ alkyl, amino C₁₋₄ alkyl, carboxyl C₁₋₄ alkyl or halogenated C₁₋₄ alkoxy;
each Q₁ is independently selected from deuterium, halogen, or C₁₋₄ alkoxy, C₁₋₄ alkylamino, di(C₁₋₄ alkyl)amino, halogenated C₁₋₄ alkyl, halogenated C₁₋₄ alkoxy, hydroxy C₁₋₄ alkyl, amino C₁₋₄ alkyl, carboxyl C₁₋₄ alkyl, —(L)ₘ-C₁₋₄ alkyl, or —(L)ₘ-3-6 membered cycloalkyl optionally substituted by 1-3 substituents Q₂; and each Q₂ is independently selected from deuterium, halogen, carboxyl, hydroxyl, cyano, nitro, amino, C₁₋₄ alkyl, hydroxy C₁₋₄ alkyl, carboxyl C₁₋₄ alkyl, C₁₋₄ alkylamino, di(C₁₋₄ alkyl)amino, C₁₋₄ alkoxy, halogenated C₁₋₄ alkyl and halogenated C₁₋₄ alkoxy;
each L is independently selected from —CR^{a}R^{b}— or —O—;
each R^{a} and each R^{b} are each independently selected from deuterium, hydrogen, or optionally deuterated C₁₋₄ alkyl, C₁₋₄ alkoxy, C₁₋₄ alkylamino, di(C₁₋₄ alkyl)amino, halogenated C₁₋₄ alkyl, halogenated C₁₋₄ alkoxy, hydroxy C₁₋₄ alkyl, amino C₁₋₄ alkyl and carboxyl C₁₋₄ alkyl;
each R^{c} is independently selected from deuterium, hydrogen, or optionally deuterated C₁₋₄ alkyl, halogenated C₁₋₄ alkyl and halogenated C₁₋₄ alkoxy;
each m is independently an integer of 0-2; and
each n is independently an integer of 0-4.

4. The compound or the pharmaceutically acceptable salt, ester, deuterated compound or stereoisomer thereof according to claim 1,
wherein:
X₁, X₂, X₃ and X₄ are each independently selected from N;
R¹, R² and R³ are each independently selected from phenyl, furyl, thienyl, pyrrolyl, thiazolyl, isothiazolyl, thiadiazolyl, oxazolyl, isoxazolyl, oxadiazole, imidazolyl, pyrazolyl, 1,2,3-triazolyl, 1,2,4-triazolyl, pyridyl, 2-pyridone, 4-pyridone, pyrimidinyl, pyridazinyl, pyrazinyl, 1,2,3-triazolyl, 1,3,5-triazinyl and 1,2,4,5 -tetrazinyl, each of which is optionally substituted by 1-3 Q₁;
R⁴ and R⁵ are each independently selected from deuterium, hydrogen, or from methyl, ethyl, propyl, isopropyl, butyl, isobutyl, sec-butyl, tert-butyl, methoxy, ethoxy, propoxy, isopropoxy, methylamino, dimethylamino, monofluoromethyl, difluoromethyl, trifluoromethyl, hydroxymethyl, hydroxyethyl, hydroxypropyl, hydroxybutyl, aminomethyl, carboxymethyl, carboxyethyl, monofluoromethoxy, difluoromethoxy and trifluoromethoxy, each of which is optionally deuterated;
each Q₁ is independently selected from deuterium, fluorine, chlorine, bromine, iodine, or from methoxy, ethoxy, propoxy, isopropoxy, methylamino, dimethylamino, monofluoromethyl, difluoromethyl, trifluoromethyl, monofluoromethoxy, difluoromethoxy, trifluoromethoxy, hydroxymethyl, hydroxyethyl, hydroxypropyl, hydroxybutyl, aminomethyl, carboxymethyl, carboxyethyl, —(L)ₘ-C₁₋₄ alkyl, or —(L)ₘ-3-6 membered cycloalkyl, each of which is optionally substituted by 1-3 substituents Q₂, and each Q₂ is independently selected from deuterium, halogen, carboxyl, hydroxyl, cyano, nitro, amino, methyl, ethyl, propyl, isopropyl, butyl, isobutyl, sec-butyl, tert-butyl, methoxy, ethoxy, propoxy, isopropoxy, hydroxymethyl, hydroxyethyl, hydroxypropyl, hydroxybutyl, carboxymethyl, carboxyethyl, methylamino, dimethylamino, monofluoromethyl, difluoromethyl, trifluoromethyl, monofluoromethoxy, difluoromethoxy and trifluoromethoxy;
each L is independently selected from —CR^{a}R^{b}—;
each R^{a} and each R^{b} are each independently selected from deuterium, hydrogen, or from methyl, ethyl, propyl, isopropyl, butyl, isobutyl, sec-butyl, tert-butyl, methoxy, ethoxy, propoxy or isopropoxy, each of which is optionally deuterated;
each m is independently 0,1 or 2; and
each n is independently 0, 1, 2 or 3.

5. The compound or the pharmaceutically acceptable salt, ester, deuterated compound or stereoisomer thereof according to claim 1,
wherein:
R¹ and R³ are each independently selected from 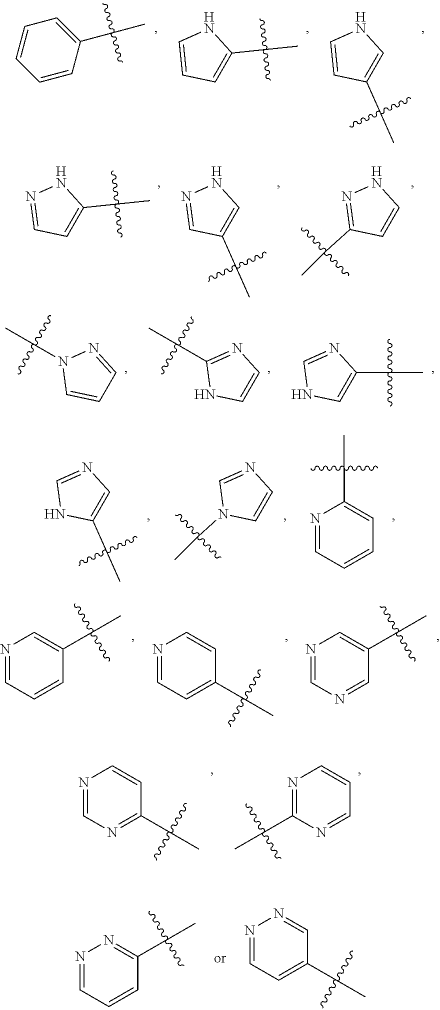
optionally substituted by 1-3 Q₁;
and/or R² is selected from phenyl or pyridyl optionally substituted by 1-3 Q₁.

6. The compound or the pharmaceutically acceptable salt, ester, deuterated compound or stereoisomer thereof according to claim 1, having a structure of formula (II-1), 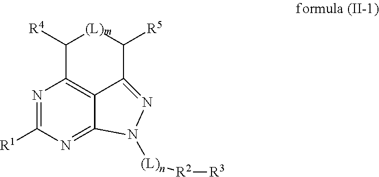
wherein:
each L, each R^{a}, each R^{b}, each R^{c}, R¹, R², R³, R⁴, R⁵, each Q₁, each Q₂, m and n are as defined in claim 1.

7. The compound or the pharmaceutically acceptable salt, ester, deuterated compound or stereoisomer thereof according to claim 1, having a structure of formula (II-2), 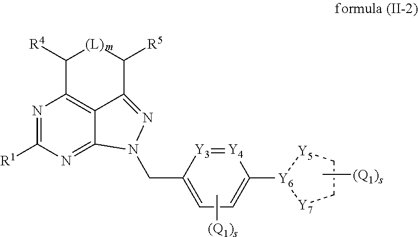
wherein, Y₃, Y₄ and Y₆ are each independently selected from N, C or CR^{a};
Y₅ and Y₇ are each independently selected from N, NR^{c}, CR^{a}R^{b} or CR^{a}; [symbol]
and - is each independently selected from a single bond or a double bond, and two adjacent bonds are incapable of being double bonds at the same time;
each s is independently selected from an integer of 0-2; and
each Q₁, each Q₂, each L, m, R¹, R⁴, R⁵, each R^{a}, each R^{b} and each R^{c} are as defined in claim 1.

8. The compound or the pharmaceutically acceptable salt, ester, deuterated compound or stereoisomer thereof according to claim 1, having a structure of formula (II-5), 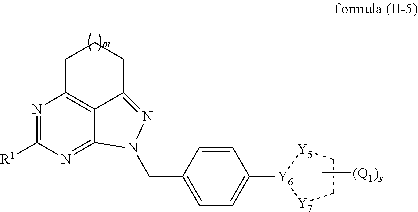
wherein:
R¹ is selected from phenyl or 5-6 membered nitrogen-containing heteroaryl optionally substituted by 1-3 Q₁;
each Q₁ is independently selected from deuterium, hydrogen, or from C₁₋₆ alkyl, C₁₋₆ alkoxy, halogenated C₁₋₆ alkyl, halogenated C₁₋₆ alkoxy, and 3-6 membered cycloalkyl, each of which is optionally substituted by 1-3 substituents Q₂; and each Q₂ is independently selected from deuterium, C₁₋₆ alkyl, C₁₋₆ alkoxy, halogenated C₁₋₆ alkyl and halogenated C₁₋₆ alkoxy; 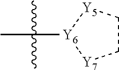
is selected from the following structures: 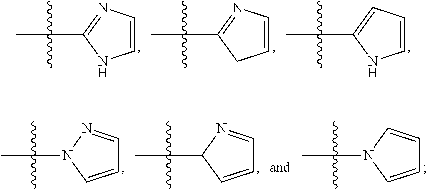 [SMILES: *c1ncc[nH]1 ; *C1=NC=CC1 ; *c1ccc[nH]1 ; *n1cccn1 ; *C1C=CC=N1 ; *n1cccc1]
m is an integer of 1 or 2; and
each s is independently an integer of 0, 1 or 2.

9. The compound or the pharmaceutically acceptable salt, ester, deuterated compound or stereoisomer thereof according to claim 1, having a structure of formula (III-1), 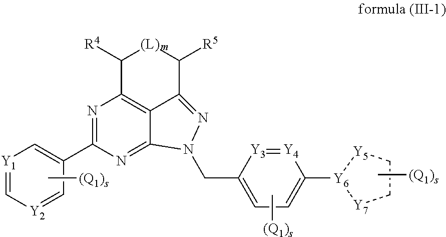
wherein, Y₁, Y₂, Y₃, Y₄ and Y₆ are each independently selected from N, C or CR^{a};
Y₅ and Y₇ are each independently selected from N, NR^{c}, C, CR^{a}R^{b} or CR^{a}; [symbol]
and - is each independently selected from a single bond or a double bond, and two adjacent bonds are incapable of being double bonds at the same time;
each s is independently selected from an integer of 0-3; and
each Q₁, m, each L, each R^{a}, each R^{b}, each R^{c}, R⁴ and R⁵ are as defined in claim 1.

10. The compound or the pharmaceutically acceptable salt, ester, deuterated compound or stereoisomer thereof according to claim 1, having a structure of formula (IV-1′), 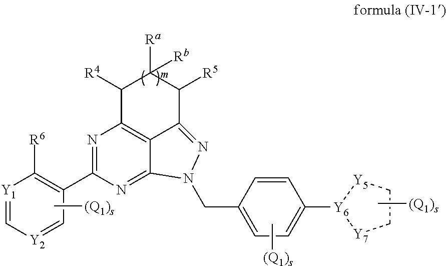
wherein, R⁶ is selected from hydrogen, or optionally deuterated C₁₋₆ alkyl, C₁₋₆ alkoxy, halogenated C₁₋₆ alkyl and halogenated C₁₋₆ alkoxy;
Y₁ and Y₂ are each independently selected from N, C or CR^{a};
Y₆ is selected from N, C or CR^{a};
Y₅ and Y₇ are each independently selected from N, NR^{c}, CR^{a}R^{b} or CR^{a}; [symbol]
and - is each independently selected from a single bond or a double bond, and two adjacent bonds are incapable of being double bonds at the same time;
each s is independently selected from an integer of 0-2; and
each Q₁, m, R⁴, R⁵, each R^{a}, each R^{b} are as defined in claim 1.

11. The compound or the pharmaceutically acceptable salt, ester, deuterated compound or stereoisomer thereof according to claim 10, wherein:
R⁶ is selected from deuterated methoxy, deuterated ethoxy, deuterated propoxy or deuterated isopropoxy, and a number of deuteration is 1, 2 or 3.

12. The compound or the pharmaceutically acceptable salt, ester, deuterated compound or stereoisomer thereof according to claim 1, having a structure of formula (V-1), 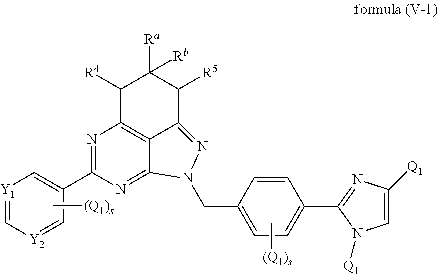
wherein, Y₁, Y₂ are each independently selected from N, C or CR^{a};
each s is independently selected from an integer of 0-3; and
each Q₁, each R^{a}, each R^{b}, R⁴ and R⁵ are as defined in claim 1.

13. The compound or the pharmaceutically acceptable salt, ester, deuterated compound or stereoisomer thereof according to claim 1, having a structure of formula (VI), 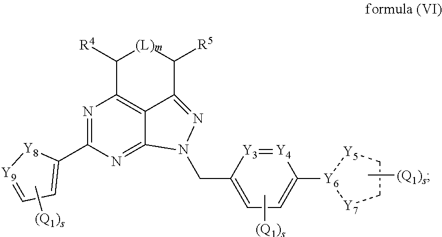
wherein, Y₈ is selected from N, NR^{c}, C, CR^{a}R^{b} or CR^{a};
Y₉ is selected from N, C or CR^{a};
Y₃, Y₄ and Y₆ are each independently selected from N, C or CR^{a};
Y₅ and Y₇ are each independently selected from N, NR^{c}, CR^{a}R^{b} or CR^{a}; [symbol]
and - is each independently selected from a single bond or a double bond, and two adjacent bonds are incapable of being double bonds at the same time;
each s is independently selected from an integer of 0-2; and
each Q₁, each L, m, R⁴, and R⁵ are as defined in claim 1.

14. The compound or the pharmaceutically acceptable salt, ester, deuterated compound or stereoisomer thereof according to claim 1, having a structure of formula (VIII-1′), 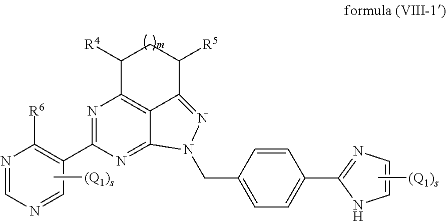
wherein, each Q₁ is independently selected from deuterium, halogen, or C₁₋₄ alkyl, C₁₋₄ alkoxy, C₁₋₄ alkylamino, di(C₁₋₄ alkyl)amino, halogenated C₁₋₄ alkyl, halogenated C₁₋₄ alkoxy, hydroxy C₁₋₄ alkyl, amino C₁₋₄ alkyl, carboxyl C₁₋₄ alkyl or 3-6 membered cycloalkyl optionally substituted by 1-3 substituents Q₂;
each Q₂ is independently selected from deuterium, hydrogen, C₁₋₄ alkyl, C₁₋₄ alkoxy, halogenated C₁₋₄ alkyl or halogenated C₁₋₄ alkoxy;
R⁴ and R⁵ are each independently selected from deuterium, hydrogen, cyano, halogen, or optionally deuterated C₁₋₆ alkyl, C₁₋₆ alkoxy, halogenated C₁₋₆ alkyl or halogenated C₁₋₆ alkoxy;
R⁶ is selected from hydrogen and deuterated C₁₋₄ alkoxy;
m is 1 or 2; and
each s is independently 0, 1 or 2.

15. The compound or the pharmaceutically acceptable salt, ester, deuterated compound or stereoisomer thereof according to claim 1, having the following structure:

| No. | Structure |
|---|---|
| Compound 1 | 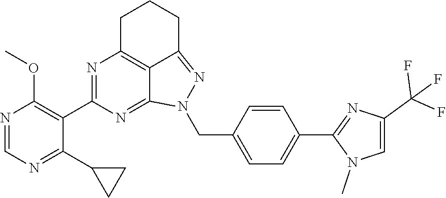 [SMILES: COc1ncnc(C2CC2)c1-c1nc2c3c(nn(Cc4ccc(-c5nc(C(F)(F)F)cn5C)cc4)c3n1)CCC2] |
| Compound 2 | 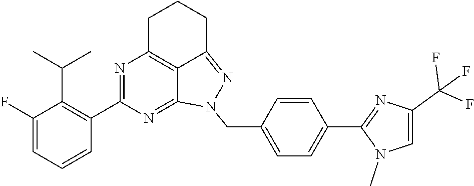 |
| Compound 3 | 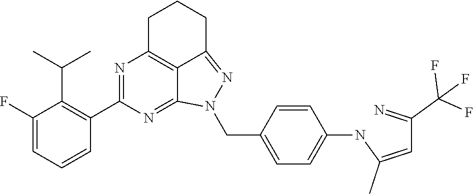 [SMILES: C/C(=C/C(=N)C(F)(F)F)Nc1ccc(Cn2nc3c4c(nc(-c5cccc(F)c5C(C)C)nc42)CCC3)cc1] |
| Compound 4 | 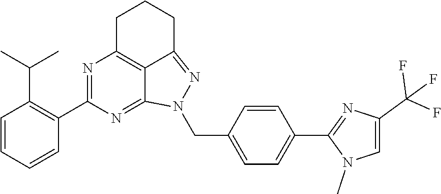 [SMILES: CC(C)c1ccccc1-c1nc2c3c(nn(Cc4ccc(-c5nc(C(F)(F)F)cn5C)cc4)c3n1)CCC2] |
| Compound 5 | 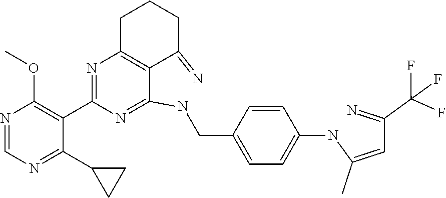 [SMILES: COc1ncnc(C2CC2)c1-c1nc2c(c(NCc3ccc(N/C(C)=C\C(=N)C(F)(F)F)cc3)n1)C(=N)CCC2] |
| Compound 6 | 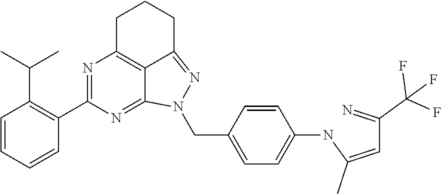 [SMILES: C/C(=C/C(=N)C(F)(F)F)Nc1ccc(Cn2nc3c4c(nc(-c5ccccc5C(C)C)nc42)CCC3)cc1] |
| Compound 7 | 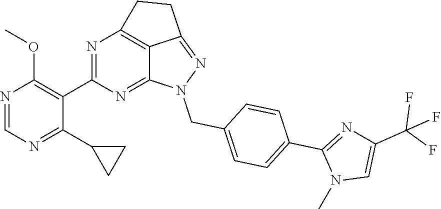 [SMILES: COc1ncnc(C2CC2)c1-c1nc2c3c(nn(Cc4ccc(-c5nc(C(F)(F)F)cn5C)cc4)c3n1)CC2] |
| Compound 8 | 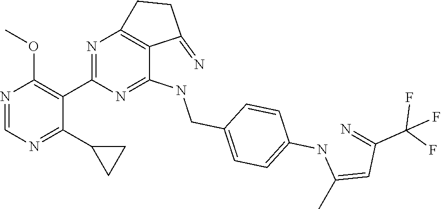 [SMILES: COc1ncnc(C2CC2)c1-c1nc2c(c(NCc3ccc(N/C(C)=C\C(=N)C(F)(F)F)cc3)n1)C(=N)CC2] |
| Compound 9 | 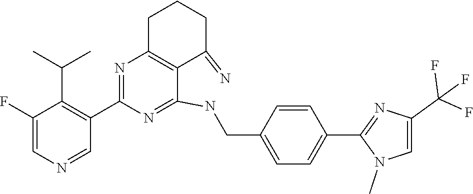 [SMILES: CC(C)c1c(F)cncc1-c1nc2c(c(NCc3ccc(-c4nc(C(F)(F)F)cn4C)cc3)n1)C(=N)CCC2] |
| Compound 10 | 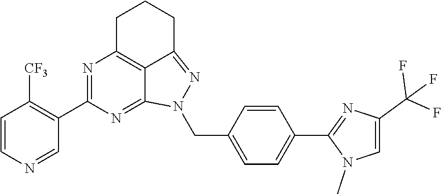 [SMILES: Cn1cc(C(F)(F)F)nc1-c1ccc(Cn2nc3c4c(nc(-c5cnccc5C(F)(F)F)nc42)CCC3)cc1] |
| Compound 11 | 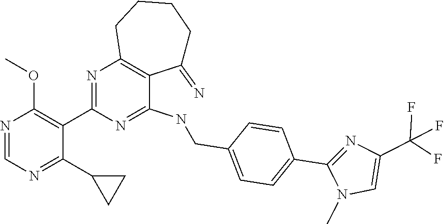 [SMILES: COc1ncnc(C2CC2)c1-c1nc2c(c(NCc3ccc(-c4nc(C(F)(F)F)cn4C)cc3)n1)C(=N)CCCC2] |
| Compound 12 | 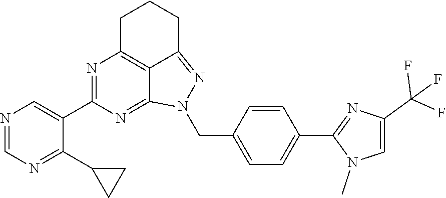 [SMILES: Cn1cc(C(F)(F)F)nc1-c1ccc(Cn2nc3c4c(nc(-c5cncnc5C5CC5)nc42)CCC3)cc1] |
| Compound 13 | 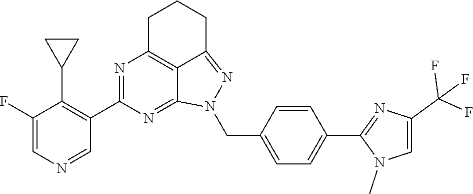 [SMILES: Cn1cc(C(F)(F)F)nc1-c1ccc(Cn2nc3c4c(nc(-c5cncc(F)c5C5CC5)nc42)CCC3)cc1] |
| Compound 14 | 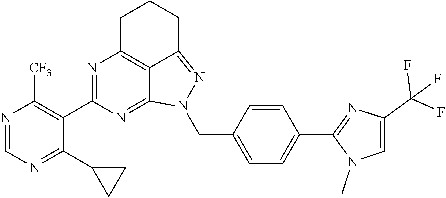 [SMILES: Cn1cc(C(F)(F)F)nc1-c1ccc(Cn2nc3c4c(nc(-c5c(C6CC6)ncnc5C(F)(F)F)nc42)CCC3)cc1] |
| Compound 15 | 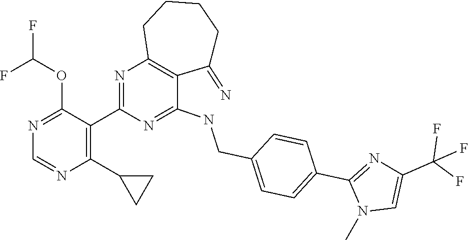 [SMILES: Cn1cc(C(F)(F)F)nc1-c1ccc(CNc2nc(-c3c(OC(F)F)ncnc3C3CC3)nc3c2C(=N)CCCC3)cc1] |
| Compound 16 | 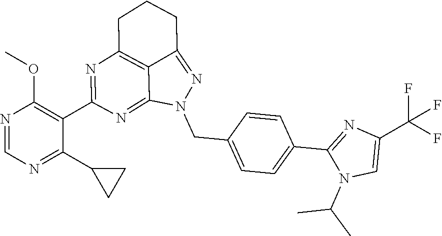 [SMILES: COc1ncnc(C2CC2)c1-c1nc2c3c(nn(Cc4ccc(-c5nc(C(F)(F)F)cn5C(C)C)cc4)c3n1)CCC2] |
| Compound 17 | 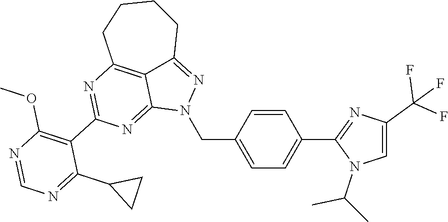 [SMILES: COc1ncnc(C2CC2)c1-c1nc2c3c(nn(Cc4ccc(-c5nc(C(F)(F)F)cn5C(C)C)cc4)c3n1)CCCC2] |
| Compound 18 | 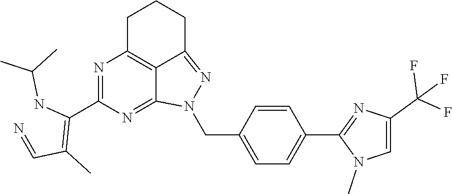 [SMILES: C/C(C=N)=C(/NC(C)C)c1nc2c3c(nn(Cc4ccc(-c5nc(C(F)(F)F)cn5C)cc4)c3n1)CCC2] |
| Compound 19 | 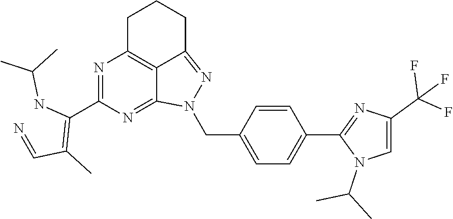 [SMILES: C/C(C=N)=C(/NC(C)C)c1nc2c3c(nn(Cc4ccc(-c5nc(C(F)(F)F)cn5C(C)C)cc4)c3n1)CCC2] |
| Compound 20 | 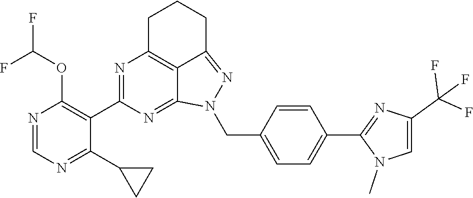 [SMILES: Cn1cc(C(F)(F)F)nc1-c1ccc(Cn2nc3c4c(nc(-c5c(OC(F)F)ncnc5C5CC5)nc42)CCC3)cc1] |
| Compound 1-1 | 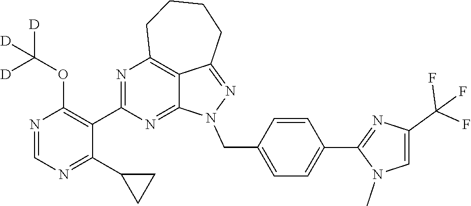 [SMILES: [2H]C([2H])([2H])Oc1ncnc(C2CC2)c1-c1nc2c3c(nn(Cc4ccc(-c5nc(C(F)(F)F)cn5C)cc4)c3n1)CCCC2] |
| Compound 5-1 | 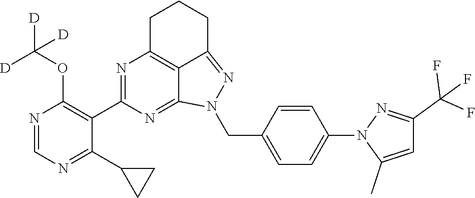 [SMILES: [2H]C([2H])([2H])Oc1ncnc(C2CC2)c1-c1nc2c3c(nn(Cc4ccc(-n5nc(C(F)(F)F)cc5C)cc4)c3n1)CCC2] |
| Compound 7-1 | 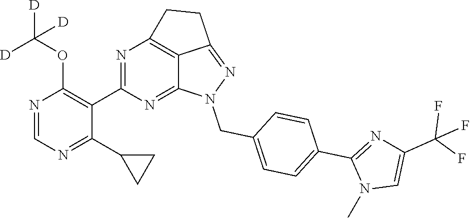 [SMILES: [2H]C([2H])([2H])Oc1ncnc(C2CC2)c1-c1nc2c3c(nn(Cc4ccc(-c5nc(C(F)(F)F)cn5C)cc4)c3n1)CC2] |
| Compound 8-1 | 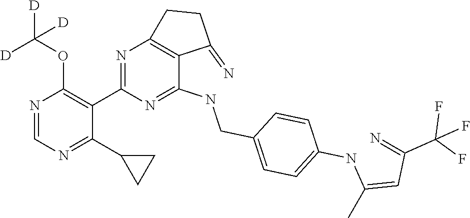 [SMILES: [2H]C([2H])([2H])Oc1ncnc(C2CC2)c1-c1nc2c(c(NCc3ccc(N/C(C)=C\C(=N)C(F)(F)F)cc3)n1)C(=N)CC2] |
| Compound 11-1 | 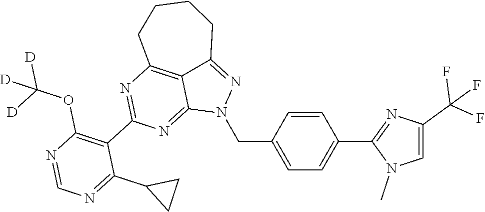 [SMILES: [2H]C([2H])([2H])Oc1ncnc(C2CC2)c1-c1nc2c3c(nn(Cc4ccc(-c5nc(C(F)(F)F)cn5C)cc4)c3n1)CCCC2] |
| Compound 16-1 | 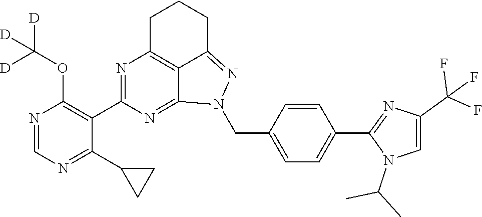 [SMILES: [2H]C([2H])([2H])Oc1ncnc(C2CC2)c1-c1nc2c3c(nn(Cc4ccc(-c5nc(C(F)(F)F)cn5C(C)C)cc4)c3n1)CCC2] |
| Compound 17-1 | 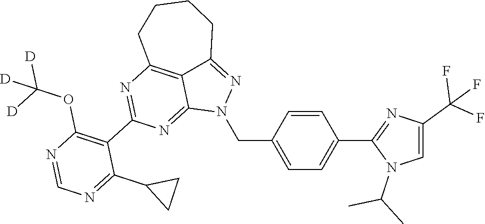 [SMILES: [2H]C([2H])([2H])Oc1ncnc(C2CC2)c1-c1nc2c3c(nn(Cc4ccc(-c5nc(C(F)(F)F)cn5C(C)C)cc4)c3n1)CCCC2] |

16. A pharmaceutical formulation, comprising the compound or the pharmaceutically acceptable salt, ester, deuterated compound or stereoisomer thereof according to claim 1, and one or more pharmaceutically acceptable carriers and/or diluents, wherein the pharmaceutical formulation is any dosage form that is clinically or pharmaceutically acceptable.

17. A pharmaceutical composition, comprising the compound or the pharmaceutically acceptable salt, ester, deuterated compound or stereoisomer thereof according to claim 1, and one or more second therapeutic active agents; optionally, the pharmaceutical composition further comprising one or more pharmaceutically acceptable carriers and/or diluents.

18. A method for manufacturing a medicament for treating and/or preventing a disease mediated by USP1 and a disease related thereto by using the compound or the pharmaceutically acceptable salt, ester, deuterated compound or stereoisomer thereof according to claim 1.

19. A method for preparing the compound or the pharmaceutically acceptable salt, ester, deuterated compound or stereoisomer thereof according to claim 1, comprising the following steps of: 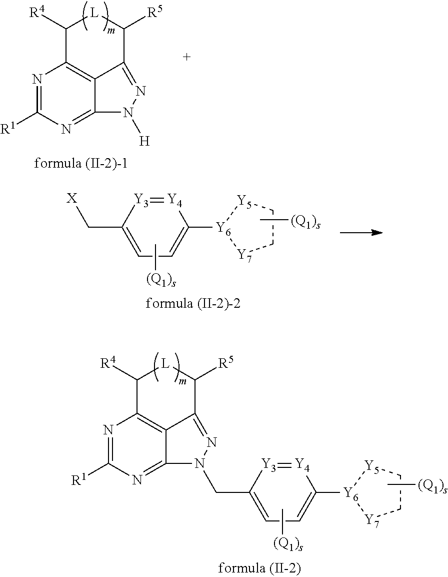
reacting the compound of formula (II-2)-1 with the compound of formula (II-2)-2 to obtain the compound of formula (II-2);
wherein, X is halogen;
Y₃, Y₄ and Y₆ are each independently selected from N, C or CR^{a};
Y₅ and Y₇ are each independently selected from N, NR^{c}, CR^{a}R^{b} or CR^{a};
each s is independently selected from an integer of 0-2;
and each Q₁, each Q₂, each L, each m, R¹, R⁴, R⁵, each R^{a}, each R^{b}, and each R^{c} are as defined in claim 1.

20. An intermediate for preparing the compound or the pharmaceutically acceptable salt, ester, deuterated compound or stereoisomer thereof according to claim 1, having the following structure: 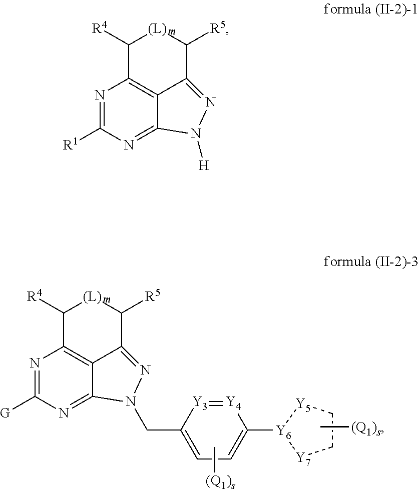
wherein, G is selected from hydrogen, hydroxyl, amino, C₁₋₆ alkylthio or C₁₋₆ alkylsulfonyl;
Y₃, Y₄ and Y₆ are each independently selected from N, C or CR^{a};
Y₅ and Y₇ are each independently selected from N, NR^{c}, CR^{a}R^{b} or CR^{a};and
R¹, each R⁴, each R⁵, each Q₁, each Q₂, each L, and each m are as defined in claim 1.
